SEQUENCE LISTING (180 sequences)

SEQ ID NO: 1 - protein, 114 aa, Homo sapiens
  MISC_FEATURE: IL-15
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 77 aa, Artificial sequence
  IL15-Ralphasushi
ITCPPPMSVEHADIWVKSYSLYSRERYICNSGFKRKAGTSSLTECVLNKATNVAHWTTPSLKCIRDPALVHQRPAPP

SEQ ID NO: 3 - DNA, 912 bp, artificial sequence
  IL-15 Ralphasushi_GSlinker_BLV1H12 light chain
atcacctgcccacctccaatgagcgtggagcacgcagacatctgggtgaagtcttacagcctgtattcccgggagagatacatctgcaactctggcttcaagcggaaggccggcaccagctccctgacagagtgcgtgctgaacaaggccaccaatgtggcccactggacaactccttccctgaaatgtattagagaccccgccctggtgcatcagagacctgccccccctggtggaggcggttcaggcggaggtggatcccaggccgtcctgaaccagccaagcagcgtctccgggtctctggggcagcgggtctcaatcacctgtagcgggtcttcctccaatgtcggcaacggctacgtgtcttggtatcagctgatccctggcagtgccccacgaaccctgatctacggcgacacatccagagcttctggggtccccgatcggttctcagggagcagatccggaaacacagctactctgaccatcagctccctgcaggctgaggacgaagcagattatttctgcgcatctgccgaggactctagttcaaatgccgtgtttggaagcggcaccacactgacagtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcataa

SEQ ID NO: 4 - protein, 524 aa, artificial sequence
  IL2 receptor subunit beta
VNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 5 - DNA, 513 bp, artificial sequence
  BLV1H 12 heavy chain
caggtccagctgagagagagcggcccttcactggtcaagccatcccagacactgagcctgacatgcacagcaagcgggttttcactgagcgacaaggcagtgggatgggtccgacaggcaccaggaaaagccctggaatggctgggcagcatcgataccggcgggaacacagggtacaatcccggactgaagagcagactgtccattaccaaggacaactctaaaagtcaggtgtcactgagcgtgagctccgtcaccacagaggatagtgcaacttactattgcacctctgtgcaccaggaaactaagaaataccagagctgtcctgacggctatcgggagagatctgattgcagtaataggccagcttgtggcacatccgactgctgtcgcgtgtctgtcttcgggaactgcctgactaccctgcctgtgtcctactcttatacctacaattatgaatggcatgtggatgtctggggacagggcctgctggtgacagtctctagtgctagc

SEQ ID NO: 6 - DNA, 1557 bp, artificial sequence
  (Sig Seq- VRegion - CH1CH2CH3) BLV1H12 V in human IgG
atgggatggtcatgtatcatcctttttctagtagcaactgcaaccggtgtacattcccaggtgcagctgcgggagtcgggccccagcctgatgaagccgtcacagaccctctccctcacctgcacggtctctggatcttcattgaacgacaagtctgtaggctgggtccgccaggctccagggaaggcgctgcagtggctcggtagtgtggacactagtggaaacacagactataacccaggcctgaaatcccggctcagcatcaccaaggacaactccaagagccgaatctctcttacagtgactggcatgacaactgaagactcggccacatactactgtacttctgtgcaccaggaaacaaaaaaataccaaagttgtccggaggattatacttataatccacgttgccctcagcagtatggttggagtgactgtgattgtatgggcgataggtttgggggttactgtcgacaggatggttgtagtaattatagttatacttacaattacgaatggcacgtcgatgtctggggccaaggactcctggtcaccgtctcctcagctagcaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 7 - DNA, 823 bp, artificial sequence
  B15 variable region plus signal peptide
gaattccaccatgggatggtcatgtatcatcctttttctagtagcaactgcaaccggagtacattcccaggtgcagctgcgcgagtcgggccccagcctggtgaagccgtcacagaccctctcgctcacctgcacggcctctggattctcattgagcgacaaggctgtaggctgggtccgccaggctccagggaaggcgctggagtggctcggtagtatagacactggtggaaacacaggctataacccaggcctgaaatcccggctcagcatcaccaaggacaactccaagagtcaagtctctctgtcagtgagcagcgtgacaactgaggactcggccacatactactgtacttctgtgcaccaggaaacaaaaaaataccaaaccggtggatcaaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctggttcaggatcctatacttacaattacgaatggcacgtcgatgtctggggccaaggactcctggtcaccgtctcctcagctagc

SEQ ID NO: 8 - DNA, 672 bp, artificial sequence
  BLV1H12 Light Chain
tcacgaattcgcaggccgtcctgaaccagccaagcagcgtctccgggtctctggggcagcgggtctcaatcacctgtagcgggtcttcctccaatgtcggcaacggctacgtgtcttggtatcagctgatccctggcagtgccccacgaaccctgatctacggcgacacatccagagcttctggggtccccgatcggttctcagggagcagatccggaaacacagctactctgaccatcagctccctgcaggctgaggacgaagcagattatttctgcgcatctgccgaggactctagttcaaatgccgtgtttggaagcggcaccacactgacagtcctggggcagcccaagagtcccccttcagtgactctgttcccaccctctaccgaggaactgaacggaaacaaggccacactggtgtgtctgatcagcgacttttaccctggatccgtcactgtggtctggaaggcagatggcagcacaattactaggaacgtggaaactacccgcgcctccaagcagtctaatagtaaatacgccgccagctcctatctgagcctgacctctagtgattggaagtccaaagggtcatatagctgcgaagtgacccatgaaggctcaaccgtgactaagactgtgaaaccatccgagtgctcctaggctagctggc

SEQ ID NO: 9 - protein, 12 aa, artificial sequence
  BLV1H 12 ascending stalk region
TSVHQETKKYQS

SEQ ID NO: 10 - protein, 12 aa, artificial sequence
  BLV1H 12 decending stalk region
SYTYNYEWHVDV

SEQ ID NO: 11 - protein, 11 aa, artificial sequence
  V2 alternative sequence
WGQGLLVTVSS

SEQ ID NO: 12 - protein, 95 aa, artificial sequence
  V1 Alternative B sequence of VH4-34_Q5RQ6E
QVQLREWGAGLLKPSETLSLTCAVYGGSFSGYYWSWIRQPPGKGLEWIGEINHSGSTNYNPSLKSRVTISVDTSKNQFSLKLSSVTAADTAVYYC

SEQ ID NO: 13 - protein, 95 aa, artificial sequence
  V1 Alternative B sequence of VH4-34_CDR1-G31DY32K_Q5RQ6E
QVQLREWGAGLLKPSETLSLTCAVYGGSFSDKYWSWIRQPPGKGLEWIGEINHSGSTNYNPSLKSRVTISVDTSKNQFSLKLSSVTAADTAVYYC

SEQ ID NO: 14 - protein, 95 aa, artificial sequence
  V1 Alternative B sequence of VH4-34_CDR2-E50S_Q5RQ6E
QVQLREWGAGLLKPSETLSLTCAVYGGSFSGYYWSWIRQPPGKGLEWIGSINHSGSTNYNPSLKSRVTISVDTSKNQFSLKLSSVTAADTAVYYC

SEQ ID NO: 15 - protein, 95 aa, artificial sequence
  synthesized V1 Alternative B sequence of VH4-34_CDR1-G31DY32K_CDR2-E50S_Q5RQ6E
QVQLREWGAGLLKPSETLSLTCAVYGGSFSDKYWSWIRQPPGKGLEWIGSINHSGSTNYNPSLKSRVTISVDTSKNQFSLKLSSVTAADTAVYYC

SEQ ID NO: 16 - protein, 95 aa, artificial sequence
  synthesized V1 Alternative B sequence of VH4-34_CDR1-Cow_Q5RQ6E
QVQLREWGAGLLKPSETLSLTCTASGFSLSDKAVGWIRQPPGKGLEWIGEINHSGSTNYNPSLKSRVTISVDTSKNQFSLKLSSVTAADTAVYYC

SEQ ID NO: 17 - protein, 95 aa, artificial sequence
  synthesized V1 Alternative B sequence of VH4-34_CDR2-Cow_Q5RQ6E
QVQLREWGAGLLKPSETLSLTCAVYGGLGSIDTGGNTGSFSGYYWSWIRQPPGKGLEWYNPSLKSRVTISVDTSKNQFSLKLSSVTAADTAVYYC

SEQ ID NO: 18 - protein, 95 aa, artificial sequence
  synthesized V1 Alternative B sequence of VH4-34_CDR1-Cow_CDR2-E50S_Q5RQ6E
QVQLREWGAGLLKPSETLSLTCTASGFSLSDKAVGWIRQPPGKGLEWIGSINHSGSTNYNPSLKSRVTISVDTSKNQFSLKLSSVTAADTAVYYC

SEQ ID NO: 19 - protein, 95 aa, artificial sequence
  synthesized V1 Alternative N sequence of VH4-34_CDR1-Cow_CDR2-Cow_Q5RQ6E
QVQLREWGAGLLKPSETLSLTCTASGFSLSDKAVGWIRQPPGKGLEWLGSIDTGGNTGYNPSLKSRVTISVDTSKNQFSLKLSSVTAADTAVYYC

SEQ ID NO: 20 - protein, 11 aa, artificial sequence
  V2 alternative sequence
WGHGTAVTVSS

SEQ ID NO: 21 - protein, 11 aa, artificial sequence
  V2 alternative sequence
WGKGTTVTVSS

SEQ ID NO: 22 - protein, 11 aa, artificial sequence
  V2 alternative sequence
WGKGTTVTVSS

SEQ ID NO: 23 - protein, 11 aa, artificial sequence
  V2 alternative sequence
WGRGTLVTVSS

SEQ ID NO: 24 - protein, 11 aa, artificial sequence
  V2 alternative sequence
WGKGTTVTVSS

SEQ ID NO: 25 - protein, 61 aa, artificial sequence
  Synthesized ultralong CDR3 sequence (BLV1H12)
SVHQETKKYQSCPDGYRERSDCSNRPACGTSDCCRVSVFGNCLTTLPVSYSYTYNYEWHVD

SEQ ID NO: 26 - protein, 274 aa, artificial sequence
  BLV1H12 Heavy Chain
QVQLRESGPSLVKPSQTLSLTCTASGFSLSDKAVGWVRQAPGKALEWLGSIDTGGNTGYNPGLKSRLSITKDNSKSQVSLSVSSVTTEDSATYYCTSVHQETKKYQSCPDGYRERSDCSNRPACGTSDCCRVSVFGNCLTTLPVSYSYTYNYEWHVDVWGQGLLVTVSSASTTAPKVYPLSSCCGDKSSSTVTLGCLVSSYMPEPVTVTWNSGALKSGVHTFPAVLQSSGLYSLSSMVTVPGSTSGQTFTCNVAHPASSTKVDKAVEPKSCDGS

SEQ ID NO: 27 - protein, 216 aa, artificial sequence
  BLV1H12 Light Chain
QAVLNQPSSVSGSLGQRVSITCSGSSSNVGNGYVSWYQLIPGSAPRTLIYGDTSRASGVPDRFSGSRSGNTATLTISSLQAEDEADYFCASAEDSSSNAVFGSGTTLTVLGQPKSPPSVTLFPPSTEELNGNKATLVCLISDFYPGSVTVVWKADGSTITRNVETTRASKQSNSKYAASSYLSLTSSDWKSKGSYSCEVTHEGSTVTKTVKPSECS

SEQ ID NO: 28 - protein, 269 aa, artificial sequence
  BLV5B8 heavy chain
QVQLRESGPSLVQPSQTLSLTCTASGFSLSDKAVGWVRQAPGKALEWLGSIDTGGSTGYNPGLKSRLSITKDNSKSQVSLSVSSVTTEDSATYYCTTVHQETRKTCSDGYIAVDSCGRGQSDGCVNDCNSCYYGWRNCRRQPAIHSYEFHVDAWGRGLLVTVSSASTTAPKVYPLSSCCGDKSSSTVTLGCLVSSYMPEPVTVTWNSGALKSGVHTFPAVLQSSGLYSLSSMVTVPGSTSGQTFTCNVAHPASSTKVDKAVEPKSCDGS

SEQ ID NO: 29 - protein, 216 aa, artificial sequence; identical to SEQ ID NO: 27
  BLV5B8 light chain

SEQ ID NO: 30 - protein, 56 aa, artificial sequence
  BLV5B8 CDR3
TVHQETRKTCSDGYIAVDSCGRGQSDGCVNDCNSCYYGWRNCRRQPAIHSYEFHVD

SEQ ID NO: 31 - protein, 57 aa, artificial sequence
  BLV5D3 CDR3
SVTQRTHVSRSCPDGCSDGDGCVDGCCCSAYRCYTPGVRDLSCTSYSITYTYEWNVD

SEQ ID NO: 32 - protein, 58 aa, artificial sequence
  BLV8C11 CDR3
TVHQKTTRKTCCSDAYRYDSGCGSGCDCCGADCYVFGACTFGLDSSYSYIYIYQWYVD

SEQ ID NO: 33 - protein, 56 aa, artificial sequence
  BF4E9 CDR3
TVHQIFCPDGYSYGYGCGYGYGCSGYDCYGYGGYGYGGYGGYSSYSYSYSYEYYGD

SEQ ID NO: 34 - protein, 48 aa, artificial sequence
  BF1H1 CDR3
TVHPSPDGYSYGYGCGYGYGCSGYDCYGYGGYGYGGYGGYSSYSYSYS

SEQ ID NO: 35 - protein, 48 aa, artificial sequence
  F18 CDR3
TVHQIRCPDGYGYGYGCGYGSYGYSGYDCYGYGGYGGYGGYGGYSSYS

SEQ ID NO: 36 - protein, 5 aa, artificial sequence
  ASCENDING STALK STRAND
TTVHQ

SEQ ID NO: 37 - protein, 5 aa, artificial sequence
  ASCENDING STALK STRAND
TSVHQ

SEQ ID NO: 38 - protein, 5 aa, artificial sequence
  ASCENDING STALK STRAND
SSVTQ

SEQ ID NO: 39 - protein, 5 aa, artificial sequence
  ASCENDING STALK STRAND
STVHQ

SEQ ID NO: 40 - protein, 5 aa, artificial sequence
  ASCENDING STALK STRAND
ATVRQ

SEQ ID NO: 41 - protein, 5 aa, artificial sequence
  ASCENDING STALK STRAND
TTVYQ

SEQ ID NO: 42 - protein, 5 aa, artificial sequence
  ASCENDING STALK STRAND
SPVHQ

SEQ ID NO: 43 - protein, 5 aa, artificial sequence
  ASCENDING STALK STRAND
ATVYQ

SEQ ID NO: 44 - protein, 20 aa, artificial sequence
  ASCENDING STALK STRAND
ASCENDINGSTALKSTRAND

SEQ ID NO: 45 - protein, 5 aa, artificial sequence
  ASCENDING STALK STRAND
TNVHQ

SEQ ID NO: 46 - protein, 5 aa, artificial sequence
  ASCENDING STALK STRAND
ATVHQ

SEQ ID NO: 47 - protein, 5 aa, artificial sequence
  ASCENDING STALK STRAND
STVYQ

SEQ ID NO: 48 - protein, 5 aa, artificial sequence
  ASCENDING STALK STRAND
TIVHQ

SEQ ID NO: 49 - protein, 5 aa, artificial sequence
  ASCENDING STALK STRAND
AIVYQ

SEQ ID NO: 50 - protein, 5 aa, artificial sequence
  ASCENDING STALK STRAND
TTVFQ

SEQ ID NO: 51 - protein, 5 aa, artificial sequence
  ASCENDING STALK STRAND
AAVFQ

SEQ ID NO: 52 - protein, 5 aa, artificial sequence
  ASCENDING STALK STRAND
GTVHQ

SEQ ID NO: 53 - protein, 5 aa, artificial sequence
  ASCENDING STALK STRAND
ASVHQ

SEQ ID NO: 54 - protein, 5 aa, artificial sequence
  ASCENDING STALK STRAND
TAVFQ

SEQ ID NO: 55 - protein, 5 aa, artificial sequence
  ASCENDING STALK STRAND
ATVFQ

SEQ ID NO: 56 - protein, 5 aa, artificial sequence
  ASCENDING STALK STRAND
AAAHQ

SEQ ID NO: 57 - protein, 4 aa, artificial sequence
  ASCENDING STALK STRAND
VWYQ

SEQ ID NO: 58 - protein, 5 aa, artificial sequence
  ASCENDING STALK STRAND
GTVFQ

SEQ ID NO: 59 - protein, 5 aa, artificial sequence
  ASCENDING STALK STRAND
TAVHQ

SEQ ID NO: 60 - protein, 5 aa, artificial sequence
  ASCENDING STALK STRAND
ITVHQ

SEQ ID NO: 61 - protein, 5 aa, artificial sequence
  ASCENDING STALK STRAND
ITAHQ

SEQ ID NO: 62 - protein, 5 aa, artificial sequence
  ASCENDING STALK STRAND
VTVHQ

SEQ ID NO: 63 - protein, 5 aa, artificial sequence
  ASCENDING STALK STRAND
AAVHQ

SEQ ID NO: 64 - protein, 5 aa, artificial sequence
  ASCENDING STALK STRAND
GTVYQ

SEQ ID NO: 65 - protein, 5 aa, artificial sequence
  ASCENDING STALK STRAND
TTVLQ

SEQ ID NO: 66 - protein, 5 aa, artificial sequence
  ASCENDING STALK STRAND
TTTHQ

SEQ ID NO: 67 - protein, 5 aa, artificial sequence
  ASCENDING STALK STRAND
TTDYQ

SEQ ID NO: 68 - protein, 99 aa, artificial sequence
  Human heavy chain variable region sequence VH4-39
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCAR

SEQ ID NO: 69 - protein, 96 aa, artificial sequence
  Human heavy chain variable region sequence 4-59*03
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCA

SEQ ID NO: 70 - protein, 97 aa, artificial sequence
  Human heavy chain variable region sequence 4-34*02
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCAR

SEQ ID NO: 71 - protein, 97 aa, artificial sequence
  Human heavy chain variable region sequence 4-34*09
QVQLQESGPGLVKPSQTLSLTCAVYGGSFSGYYWSWIRQPPGKGLEWIGEINHSGSTNYNPSLKSRVTISVDTSKNQFSLKLSSVTAADTAVYYCAR

SEQ ID NO: 72 - protein, 11 aa, artificial sequence
  ASCENDING STALK STRAND
TSVHQETKKYQ

SEQ ID NO: 73 - protein, 9 aa, artificial sequence
  ASCENDING STALK STRAND
VHQETKKYQ

SEQ ID NO: 74 - protein, 6 aa, artificial sequence
  ASCENDING STALK STRAND
IHSYEF

SEQ ID NO: 75 - protein, 4 aa, artificial sequence
  ASCENDING STALK STRAND
SYEF

SEQ ID NO: 76 - protein, 6 aa, artificial sequence
  DESCENDING STALK STRAND
YTYNYE

SEQ ID NO: 77 - protein, 7 aa, artificial sequence
  DESCENDING STALK STRAND
YTYNYEW

SEQ ID NO: 78 - protein, 8 aa, artificial sequence
  DESCENDING STALK STRAND
SYTYNYEW

SEQ ID NO: 79 - protein, 6 aa, artificial sequence
  DESCENDING STALK STRAND
TYNYEW

SEQ ID NO: 80 - protein, 4 aa, artificial sequence
  DESCENDING STALK STRAND
SYTY

SEQ ID NO: 81 - protein, 14 aa, artificial sequence
  ASCENDING STALK STRAND
GSKHRLRDYFLYNE

SEQ ID NO: 82 - protein, 13 aa, artificial sequence
  ASCENDING STALK STRAND
GSKHRLRDYFLYN

SEQ ID NO: 83 - protein, 12 aa, artificial sequence
  ASCENDING STALK STRAND
GSKHRLRDYFLY

SEQ ID NO: 84 - protein, 11 aa, artificial sequence
  ASCENDING STALK STRAND
GSKHRLRDYFL

SEQ ID NO: 85 - protein, 10 aa, artificial sequence
  ASCENDING STALK STRAND
GSKHRLRDYF

SEQ ID NO: 86 - protein, 9 aa, artificial sequence
  ASCENDING STALK STRAND
GSKHRLRDY

SEQ ID NO: 87 - protein, 8 aa, artificial sequence
  ASCENDING STALK STRAND
GSKHRLRD

SEQ ID NO: 88 - protein, 13 aa, artificial sequence
  ASCENDING STALK STRAND
EAGGPDYRNGYNY

SEQ ID NO: 89 - protein, 12 aa, artificial sequence
  ASCENDING STALK STRAND
EAGGPDYRNGYN

SEQ ID NO: 90 - protein, 11 aa, artificial sequence
  ASCENDING STALK STRAND
EAGGPDYRNGY

SEQ ID NO: 91 - protein, 10 aa, artificial sequence
  ASCENDING STALK STRAND
EAGGPDYRNG

SEQ ID NO: 92 - protein, 9 aa, artificial sequence
  ASCENDING STALK STRAND
EAGGPDYRN

SEQ ID NO: 93 - protein, 8 aa, artificial sequence
  ASCENDING STALK STRAND
EAGGPDYR

SEQ ID NO: 94 - protein, 7 aa, artificial sequence
  ASCENDING STALK STRAND
EAGGPDY

SEQ ID NO: 95 - protein, 6 aa, artificial sequence
  ASCENDING STALK STRAND
EAGGPD

SEQ ID NO: 96 - protein, 13 aa, artificial sequence
  ASCENDING STALK STRAND
EAGGPIWHDDVKY

SEQ ID NO: 97 - protein, 12 aa, artificial sequence
  ASCENDING STALK STRAND
EAGGPIWHDDVK

SEQ ID NO: 98 - protein, 11 aa, artificial sequence
  ASCENDING STALK STRAND
EAGGPIWHDDV

SEQ ID NO: 99 - protein, 10 aa, artificial sequence
  ASCENDING STALK STRAND
EAGGPIWHDD

SEQ ID NO: 100 - protein, 9 aa, artificial sequence
  ASCENDING STALK STRAND
EAGGPIWHD

SEQ ID NO: 101 - protein, 8 aa, artificial sequence
  ASCENDING STALK STRAND
EAGGPIWH

SEQ ID NO: 102 - protein, 7 aa, artificial sequence
  ASCENDING STALK STRAND
EAGGPIW

SEQ ID NO: 103 - protein, 6 aa, artificial sequence
  ASCENDING STALK STRAND
EAGGPI

SEQ ID NO: 104 - protein, 11 aa, artificial sequence
  ASCENDING STALK STRAND
GTDYTIDDQGI

SEQ ID NO: 105 - protein, 10 aa, artificial sequence
  ASCENDING STALK STRAND
GTDYTIDDQG

SEQ ID NO: 106 - protein, 9 aa, artificial sequence
  ASCENDING STALK STRAND
GTDYTIDDQ

SEQ ID NO: 107 - protein, 8 aa, artificial sequence
  ASCENDING STALK STRAND
GTDYTIDD

SEQ ID NO: 108 - protein, 7 aa, artificial sequence
  ASCENDING STALK STRAND
GTDYTID

SEQ ID NO: 109 - protein, 6 aa, artificial sequence
  ASCENDING STALK STRAND
GTDYTI

SEQ ID NO: 110 - protein, 11 aa, artificial sequence
  ASCENDING STALK STRAND
DKGDSDYDYNL

SEQ ID NO: 111 - protein, 10 aa, artificial sequence
  ASCENDING STALK STRAND
DKGDSDYDYN

SEQ ID NO: 112 - protein, 9 aa, artificial sequence
  ASCENDING STALK STRAND
DKGDSDYDY

SEQ ID NO: 113 - protein, 8 aa, artificial sequence
  ASCENDING STALK STRAND
DKGDSDYD

SEQ ID NO: 114 - protein, 7 aa, artificial sequence
  ASCENDING STALK STRAND
DKGDSDY

SEQ ID NO: 115 - protein, 6 aa, artificial sequence
  ASCENDING STALK STRAND
DKGDSD

SEQ ID NO: 116 - protein, 17 aa, artificial sequence
  ASCENDING STALK STRAND
YGPNYEEWGDYLATLDV

SEQ ID NO: 117 - protein, 16 aa, artificial sequence
  ASCENDING STALK STRAND
GPNYEEWGDYLATLDV

SEQ ID NO: 118 - protein, 15 aa, artificial sequence
  ASCENDING STALK STRAND
PNYEEWGDYLATLDV

SEQ ID NO: 119 - protein, 14 aa, artificial sequence
  ASCENDING STALK STRAND
NYEEWGDYLATLDV

SEQ ID NO: 120 - protein, 13 aa, artificial sequence
  ASCENDING STALK STRAND
YEEWGDYLATLDV

SEQ ID NO: 121 - protein, 12 aa, artificial sequence
  ASCENDING STALK STRAND
EEWGDYLATLDV

SEQ ID NO: 122 - protein, 15 aa, artificial sequence
  DESCENDING STALK STRAND
YDFYDGYYNYHYMDV

SEQ ID NO: 123 - protein, 14 aa, artificial sequence
  DESCENDING STALK STRAND
DFYDGYYNYHYMDV

SEQ ID NO: 124 - protein, 13 aa, artificial sequence
  DESCENDING STALK STRAND
FYDGYYNYHYMDV

SEQ ID NO: 125 - protein, 12 aa, artificial sequence
  DESCENDING STALK STRAND
YDGYYNYHYMDV

SEQ ID NO: 126 - protein, 11 aa, artificial sequence
  DESCENDING STALK STRAND
DGYYNYHYMDV

SEQ ID NO: 127 - protein, 10 aa, artificial sequence
  DESCENDING STALK STRAND
GYYNYHYMDV

SEQ ID NO: 128 - protein, 9 aa, artificial sequence
  DESCENDING STALK STRAND
YYNYHYMDV

SEQ ID NO: 129 - protein, 15 aa, artificial sequence
  DESCENDING STALK STRAND
YDFNDGYYNYHYMDV

SEQ ID NO: 130 - protein, 14 aa, artificial sequence
  DESCENDING STALK STRAND
DFYDGYYNYHYMDV

SEQ ID NO: 131 - protein, 13 aa, artificial sequence
  DESCENDING STALK STRAND
FYDGYYNYHYMDV

SEQ ID NO: 132 - protein, 12 aa, artificial sequence
  DESCENDING STALK STRAND
YDGYYNYHYMDV

SEQ ID NO: 133 - protein, 11 aa, artificial sequence
  DESCENDING STALK STRAND
DGYYNYHYMDV

SEQ ID NO: 134 - protein, 10 aa, artificial sequence
  DESCENDING STALK STRAND
GYYNYHYMDV

SEQ ID NO: 135 - protein, 16 aa, artificial sequence
  DESCENDING STALK STRAND
QGIRYQGSGTFWYFDV

SEQ ID NO: 136 - protein, 15 aa, artificial sequence
  DESCENDING STALK STRAND
GIRYQGSGTFWYFDV

SEQ ID NO: 137 - protein, 14 aa, artificial sequence
  DESCENDING STALK STRAND
IRYQGSGTFWYFDV

SEQ ID NO: 138 - protein, 13 aa, artificial sequence
  DESCENDING STALK STRAND
RYQGSGTFWYFDV

SEQ ID NO: 139 - protein, 12 aa, artificial sequence
  DESCENDING STALK STRAND
YQGSGTFWYFDV

SEQ ID NO: 140 - protein, 11 aa, artificial sequence
  DESCENDING STALK STRAND
QGSGTFWYFDV

SEQ ID NO: 141 - protein, 10 aa, artificial sequence
  DESCENDING STALK STRAND
GSGTFWYFDV

SEQ ID NO: 142 - protein, 9 aa, artificial sequence
  DESCENDING STALK STRAND
SGTFWYFDV

SEQ ID NO: 143 - protein, 8 aa, artificial sequence
  DESCENDING STALK STRAND
GTFWYFDV

SEQ ID NO: 144 - protein, 13 aa, artificial sequence
  DESCENDING STALK STRAND
YNLGYSYFYYMDG

SEQ ID NO: 145 - protein, 12 aa, artificial sequence
  DESCENDING STALK STRAND
NLGYSYFYYMDG

SEQ ID NO: 146 - protein, 11 aa, artificial sequence
  DESCENDING STALK STRAND
LGYSYFYYMDG

SEQ ID NO: 147 - protein, 10 aa, artificial sequence
  DESCENDING STALK STRAND
GYSYFYYMDG

SEQ ID NO: 148 - protein, 9 aa, artificial sequence
  DESCENDING STALK STRAND
YSYFYYMDG

SEQ ID NO: 149 - protein, 8 aa, artificial sequence
  DESCENDING STALK STRAND
SYFYYMDG

SEQ ID NO: 150 - protein, 2 aa, artificial sequence
  Linker
GS

SEQ ID NO: 151 - protein, 3 aa, artificial sequence
  Linker
GGS

SEQ ID NO: 152 - protein, 6 aa, artificial sequence
  Linker
GGSGGS

SEQ ID NO: 153 - protein, 9 aa, artificial sequence
  Linker
GGSGGSGGS

SEQ ID NO: 154 - protein, 5 aa, artificial sequence
  Linker
GGGGS

SEQ ID NO: 155 - DNA, 662 bp, artificial sequence
  human light chain lambda region
tcacgaattcgcaggccgtcctgaaccagccaagcagcgtctccgggtctctggggcagcgggtctcaatcacctgtagcgggtcttcctccaatgtcggcaacggctacgtgtcttggtatcagctgatccctggcagtgccccacgaaccctgatctacggcgacacatccagagcttctggggtccccgatcggttctcagggagcagatccggaaacacagctactctgaccatcagctccctgcaggctgaggacgaagcagattatttctgcgcatctgccgaggactctagttcaaatgccgtgtttggaagcggcaccacactgacagtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcataa

SEQ ID NO: 156 - protein, 216 aa, artificial sequence
  human VL1-51
QSVLTQPPSVSAAPGQKVTISCSGSSSNIGNNYVSWYQQLPGTAPKLLIYDNNKRPSGIPDRFSGSKSGTSATLGITGLQTGDEADYYCASAEDSSSNAVFGSGTTLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 157 - DNA, 39 bp, artificial sequence
  BLV1H12 ascending stalk region
tgtacttctgtgcaccaggaaacaaaaaaataccaaacc

SEQ ID NO: 158 - protein, 12 aa, artificial sequence
  BLV1H12 ascending stalk region
TSVHQETKKYQT

SEQ ID NO: 159 - protein, 13 aa, artificial sequence
  BLV1H12 ascending stalk region
CTSVHQETKKYQT

SEQ ID NO: 160 - DNA, 39 bp, artificial sequence
  Descending stalk region
tcctatacttacaattacgaatggcacgtcgatgtctgg

SEQ ID NO: 161 - protein, 13 aa, artificial sequence
  Descending stalk region
SYTYNYEWHVDVW

SEQ ID NO: 162 - DNA, 117 bp, artificial sequence
  BLV1H12 knob sequence
tgtccggaggattatacttataatccacgttgccctcagcagtatggttggagtgactgtgattgtatgggcgataggtttgggggttactgtcgacaggatggttgtagtaattat

SEQ ID NO: 163 - DNA, 9 bp, artificial sequence
  Coding sequencing for N-terminal GGS linker
ggtggatca

SEQ ID NO: 164 - DNA, 9 bp, artificial sequence
  Coding sequence for C-terminal GSG linker
ggttcagga

SEQ ID NO: 165 - protein, 133 aa, Homo sapiens [sequence omitted]

SEQ ID NO: 166 - DNA, 399 bp, artificial sequence
  IL2 coding sequence
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgact

SEQ ID NO: 167 - DNA, 513 bp, artificial sequence
  Chimeric ultralong bovine heavy chain sequence
caggtgcagctgcgggagtcgggccccagcctgatgaagccgtcacagaccctctccctcacctgcacggtctctggatcttcattgaacgacaagtctgtaggctgggtccgccaggctccagggaaggcgctgcagtggctcggtagtgtggacactagtggaaacacagactataacccaggcctgaaatcccggctcagcatcaccaaggacaactccaagagccgaatctctcttacagtgactggcatgacaactgaagactcggccacatactactgtacttctgtgcaccaggaaacaaaaaaataccaaagttgtccggaggattatacttataatccacgttgccctcagcagtatggttggagtgactgtgattgtatgggcgataggtttgggggttactgtcgacaggatggttgtagtaattatagttatacttacaattacgaatggcacgtcgatgtctggggccaaggactcctggtcaccgtctcctcagctagc

SEQ ID NO: 168 - DNA, 651 bp, artificial sequence
  BLV1H12 Light Chain-
caggccgtcctgaaccagccaagcagcgtctccgggtctctggggcagcgggtctcaatcacctgtagcgggtcttcctccaatgtcggcaacggctacgtgtcttggtatcagctgatccctggcagtgccccacgaaccctgatctacggcgacacatccagagcttctggggtccccgatcggttctcagggagcagatccggaaacacagctactctgaccatcagctccctgcaggctgaggacgaagcagattatttctgcgcatctgccgaggactctagttcaaatgccgtgtttggaagcggcaccacactgacagtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcataa

SEQ ID NO: 169 - DNA, 311 bp, artificial sequence
  Human heavy chain variable region sequence 4-39
cagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatccgtagacacgtccaagaaccagttctccctgaagctgagctctgtgaccgccgcagacacggctgtgtattactgtgcgagacacacagtgagggg

SEQ ID NO: 170 - DNA, 288 bp, artificial sequence
  Human heavy chain variable region sequence 4-59*03
caggtgcagctgcaggagtcgggcccaggactggtgaagccttcggagaccctgtccctcacctgcactgtctctggtggctccatcagtagttactactggagctggatccggcagcccccagggaagggactggagtggattgggtatatctattacagtgggagcaccaactacaacccctccctcaagagtcgagtcaccatatcagtagacacgtccaagaaccaattctccctgaagctgagctctgtgaccgctgcggacacggccgtgtattactgtgcg

SEQ ID NO: 171 - DNA, 291 bp, artificial sequence
  Human heavy chain variable region sequence 4-34*09
caggtgcagctgcaggagtcgggcccaggactggtgaagccttcacagaccctgtccctcacctgcgctgtctatggtgggtccttcagtggttactactggagctggatccgccagcccccagggaagggactggagtggattggggaaatcaatcatagtggaagcaccaactacaacccgtccctcaagagtcgagttaccatatcagtagacacgtctaagaaccagttctccctgaagctgagctctgtgactgccgcggacacggccgtgtattactgtgcgaga

SEQ ID NO: 172 - DNA, 290 bp, artificial sequence
  Human heavy chain variable region sequence 4-34*02
caggtgcagctacaacagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgag

SEQ ID NO: 173 - protein, 98 aa, artificial sequence
  Human germline light chain variable region sequence VL1-47
QSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSG

SEQ ID NO: 174 - protein, 99 aa, artificial sequence
  Human germline light chain variable region sequence VL1-40*1
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSG

SEQ ID NO: 175 - protein, 98 aa, artificial sequence
  Human germline light chain variable region sequence VL1-51*01
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSA

SEQ ID NO: 176 - protein, 99 aa, artificial sequence
  Human germline light chain variable region sequence VL2-18*02
QSALTQPPSVSGSPGQSVTISCTGTSSDVGSYNRVSWYQQPPGTAPKLMIYEVSNRPSGVPDRFSGSKSGNTASLTISGLQAEDEADYYCSSYTSSSTF

SEQ ID NO: 177 - DNA, 296 bp, artificial sequence
  Human germline light chain variable region sequence VL1-47
cagtctgtgctgactcagccaccctcagcgtctgggacccccgggcagagggtcaccatctcttgttctggaagcagctccaacatcggaagtaattatgtatactggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgagtggtcc

SEQ ID NO: 178 - DNA, 299 bp, artificial sequence
  Human germline light chain variable region sequence VL1-40*1
cagtctgtgctgacgcagccgccctcagtgtctggggccccagggcagagggtcaccatctcctgcactgggagcagctccaacatcggggcaggttatgatgtacactggtaccagcagcttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagcctgagtggttc

SEQ ID NO: 179 - DNA, 296 bp, artificial sequence
  Human germline light chain variable region sequence VL1-51*01
cagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatagcagcctgagtgctgg

SEQ ID NO: 180 - DNA, 297 bp, artificial sequence
  Human germline light chain variable region sequence VL2-18*02
cagtctgccctgactcagcctccctccgtgtccgggtctcctggacagtcagtcaccatctcctgcactggaaccagcagtgacgttggtagttataaccgtgtctcctggtaccagcagcccccaggcacagcccccaaactcatgatttatgaggtcagtaatcggccctcaggggtccctgatcgcttctctgggtccaagtctggcaacacggcctccctgaccatctctgggctccaggctgaggacgaggctgattattactgcagctcatatacaagcagcagcactttc

(19) United States
(12) Patent Application Publication
(10) Pub. No.: US 2022/0380487 A1
(43) Pub. Date: Dec. 1, 2022

(54) CHIMERIC CYTOKINE MODIFIED ANTIBODIES AND METHODS OF USE THEREOF

(71) Applicant: Minotaur Therapeutics, Inc., San Diego, CA (US)

(72) Inventors: Ruiqi Huang, San Diego, CA (US); Vaughn Smider, San Diego, CA (US)

(73) Assignee: Minotaur Therapeutics, Inc., San Diego, CA (US)

(21) Appl. No.: 17/770,986

(22) PCT Filed: Oct. 23, 2020
(86) PCT No.: PCT/US2020/057209

§ 371 Date: Apr. 21, 2022

Related U.S. Application Data
(60) Provisional application No. 62/925,740, filed on Oct. 24, 2019.

(51) Int. Cl. C07K 19/00; C07K 14/54; C07K 14/55; C07K 14/715

(52) U.S. Cl. CPC C07K 19/00; C07K 14/5443; C07K 14/55; C07K 14/7155; C07K 2317/56; C07K 2319/30; A61K 38/00

(57) ABSTRACT

Provided are chimeric cytokine modified antibodies containing an ultralong CDR3, such as based on a bovine antibody sequence or a humanized sequence thereof, in which a portion of the CDR3 of the heavy chain is replaced by an interleukin (IL-15) or IL-2, and related antibodies. Among provided antibodies are chimeric IL-15 cytokine modified antibody molecules that are further linked or complexed with an extracellular portion of the IL15Rα, such as the IL15Rα sushi domain. Also provided are methods of making and using the chimeric cytokine modified antibodies.

### CROSS-REFERENCE TO RELATED APPLICATIONS

This application claims priority to U.S. provisional application 62/925,740, filed Oct. 24, 2019, entitled “CHIMERIC CYTOKINE MODIFIED ANTIBODIES AND METHODS OF USE THEREOF”, the contents of which are incorporated herein by reference in their entirety for all purposes.

### SEQUENCE LISTING

The present application is being filed along with a Sequence Listing in electronic format. The Sequence Listing is provided as a file entitled 165772000140SeqList.txt, created October 22,2020, which is 79,015 bytes in size. The information in the electronic format of the Sequence Listing is incorporated by reference in its entirety.

### FIELD

The present disclosure relates to chimeric cytokine modified antibodies containing an ultralong CDR3, such as based on a bovine antibody sequence or a humanized sequence thereof, in which a portion of the CDR3 of the heavy chain is replaced by an interleukin (IL-15) or IL-2, and related antibodies. Among the molecules of the present disclosure are chimeric IL-15 cytokine modified antibody molecules that are further linked or complexed with an extracellular portion of the IL15Rα, such as the IL15Rα sushi domain. The present disclosure also provides methods of making and using the chimeric cytokine modified antibodies.

### BACKGROUND

Antibodies are natural proteins that the vertebrate immune system forms in response to foreign substances (antigens), primarily for defense against infection. Antibodies contain complementarity determining regions (CDRs) that mediate binding to a target antigen. Some bovine antibodies have unusually long VH CDR3 sequences compared to other vertebrates, which can be up to 70 amino acids long. The long CDR3s can form unique domains that protrude from the antibody surface, thereby permitting a unique antibody platform.

Interleukin (IL) 15 and IL-2 are cytokines that stimulate the proliferation and cytotoxicity of cytotoxic T lymphocytes and natural killer (NK) cells, and thus are immunotherapeutic candidates for cancer treatment. However, such cytokines can be difficult to express as a stable soluble protein and often have a short half-life in vitro and in vivo. There remains a need for improved cytokine therapeutics, such as IL-2 or IL-15 therapeutics, particularly for use for treating cancer.

### SUMMARY

Provided herein is a chimeric cytokine modified antibody or antigen binding fragment, comprising a modified ultralong CDR3 comprising an interleukin-15 (IL-15) cytokine sequence or a biologically active portion thereof that replaces at least a portion of an ultralong CDR3 region of a heavy chain of a bovine antibody or antigen-binding fragment or a humanized sequence thereof.

In some embodiments, the IL-15 cytokine sequence is human IL-15. In some embodiments, the IL-15 cytokine sequence comprises a sequence of amino acids that exhibits at least at or about 85%, at least at or about 90%, at least at or about 92%, at least at or about 95%, at least at or about 96%, at least at or about 97%, at least at or about 98%, or at least at or about 99% sequence identity to SEQ ID NO: 1. In some embodiments, the IL-15 cytokine sequence comprises the sequence of amino acids set forth in SEQ ID NO: 1. In some embodiments, the IL-15 cytokine sequence consists of the sequence of amino acids set forth in SEQ ID NO:1.

Provided herein is a chimeric cytokine modified antibody or antigen binding fragment, comprising a modified ultralong CDR3 comprising an interleukin-2 (IL-2) cytokine sequence or a biologically active portion thereof that replaces at least a portion of an ultralong CDR3 region of a heavy chain of a bovine antibody or antigen-binding fragment or a humanized sequence thereof.

In some embodiments, the IL-2 cytokine sequence is human IL-2. In some embodiments, the IL-2 cytokine sequence comprises a sequence of amino acids that exhibits at least at or about 85%, at least at or about 90%, at least at or about 92%, at least at or about 95%, at least at or about 96%, at least at or about 97%, at least at or about 98%, or at least at or about 99% sequence identity to SEQ ID NO: 165. In some embodiments, the IL-2 cytokine sequence comprises the sequence of amino acids set forth in SEQ ID NO: 165. In some embodiments, the IL-2 cytokine sequence consists of the sequence of amino acids set forth in SEQ ID NO:165.

In some of any embodiments, the cytokine sequence replaces at least a portion of an ultralong CDR3 region of a heavy chain of a bovine antibody or antigen-binding fragment. In some embodiments, the bovine antibody or antigen-binding fragment is the bovine antibody BLV1H12 or an antigen-binding fragment thereof.

In some embodiments, the bovine antibody or antigen-binding fragment comprises a variable heavy chain amino acid sequence encoded by the sequence set forth in SEQ ID NO: 5 and a variable light chain amino acid sequence encoded by the sequence set forth in SEQ ID NO: 8. In some embodiments, the bovine antibody or antigen-binding fragment comprises a variable heavy chain amino acid sequence encoded by the sequence set forth in SEQ ID NO: 167 and a variable light chain amino acid sequence encoded by the sequence set forth in SEQ ID NO: 168.

In some embodiments, the bovine antibody or antigen-binding fragment comprises a variable heavy chain set forth in SEQ ID NO: 26 and a variable light chain set forth in SEQ ID NO: 27.

In some of any embodiments, the cytokine sequence replaces at least a portion of an ultralong CDR3 region of a heavy chain of a humanized bovine antibody or antigen-binding fragment thereof. In some embodiments, the humanized bovine antibody or antigen-binding fragment thereof comprises a heavy chain or portion thereof that is a human heavy chain germline sequence or is derived from a human heavy chain germline sequence and a light chain or a portion thereof that is a human light chain germline sequence or is derived from a human light chain germline sequence. In some embodiments, the human heavy chain germline sequence is a VH4-39, VH4-59*03, VH4-34*02 or VH4-34*09 germline sequence or is a sequence set forth in any one of SEQ ID NOS: 68-71.

In some of any embodiments, the human light chain germline sequence is a VL1-51 germline sequence or is a sequence based on the VL1-51 germline sequence comprising one or more mutations, optionally wherein the VL1-51 germline sequence is set forth in SEQ ID NO: 156. In some embodiments, the one or more mutations are selected from among: one or more of amino acid replacements S2A, T5N, P8S, A12G, A13S, and P14L based on Kabat numbering; amino acid replacements S2A, T5N, P8S, A12G, A13S, and P14L based on Kabat numbering; mutations in CDR1 comprising amino acid replacements I29V and N32G; mutations in CDR2 comprising a substitution of DNN to GDT; mutations in CDR2 comprising a substitution DNNKRP to GDTSRA; or a combination of any of the forgoing.

In some of any embodiments, the provided antibody is an antigen-binding fragment comprising a variable heavy chain and a variable light chain. In some embodiments, the antibody comprises a variable heavy chain joined to a heavy chain constant domain (CH1-CH2-CH3) and a variable light chain joined to a light chain constant domain (CL1). In some embodiments, the heavy chain constant domain is from a human IgG1. In some embodiments, the light chain constant domain is a lambda light chain region.

In some of any embodiments, the at least a portion of an ultralong CDR3 region comprises the knob region and the cytokine sequence is present between the ascending stalk domain and the descending stalk domain of the modified ultralong CDR3. In some embodiments, the cytokine sequence is linked to the ascending stalk domain and/or the descending stalk domain via a flexible linker, optionally a GGS or GSG linker. In some of any embodiments, the ascending stalk domain comprises the sequence set forth in SEQ ID NO:158 or SEQ ID NO:159. In some of any embodiments, the descending stalk domain comprises the sequence set forth in SEQ ID NO:161.

In some embodiments, the provided antibody or antigen binding fragment comprises a variable heavy chain sequence encoded by the sequence of nucleotides set forth in SEQ ID NO:7 or a sequence of nucleotides that exhibits at least at or about 85%, at least at or about 90%, at least at or about 92%, at least at or about 95%, at least at or about 96%, at least at or about 97%, at least at or about 98%, at least at or about 99% sequence identity to the nucleotide sequence set forth in SEQ ID NO:7, in which is contained a modified ultralong CDR3 containing an IL-15 sequence. In some embodiments, the provided antibody or antigen binding fragment comprises a variable heavy chain sequence encoded by the sequence of nucleotides set forth in SEQ ID NO:7. In some embodiments, the provided antibody or antigen binding fragment consists of a variable heavy chain sequence encoded by the sequence of nucleotides set forth in SEQ ID NO:7.

In some of any embodiments, the antibody or antigen binding fragment is complexed with an extracellular domain of the IL15Rα comprising the IL15Rα sushi domain. In some embodiments, the extracellular domain of the IL15Rα comprising the IL15Rα sushi domain is non-covalently associated with the IL-15 sequence. In some embodiments, the extracellular domain of the IL15Rα comprising the IL15Rα sushi domain is linked to the variable light chain. In some embodiments, the extracellular domain of the IL15Rα comprising the IL15Rα sushi domain is linked to the variable light chain via a peptide linker. In some of any embodiments, the peptide linker is a glycine linker or a glycine-serine linker, optionally wherein the linker is GS.

In some of any embodiments, the extracellular domain of the IL15Rα comprising the IL15Rα sushi domain comprises the sequence set forth in SEQ ID NO:2. In some of any embodiments, the extracellular domain of the IL15Rα comprising the IL15Rα sushi domain consists of the sequence set forth in SEQ ID NO:2.

In some embodiments, the variable light chain comprises the sequence of amino acids encoded by SEQ ID NO:3.

Provided herein are polynucleotide(s) encoding a chimeric cytokine modified antibody or antigen binding fragment of any of the preceding embodiments.

Provided herein is a polynucleotide encoding a heavy chain or a variable region thereof of a chimeric cytokine modified antibody or antigen binding fragment of any of the preceding embodiments.

Provided herein is a polynucleotide encoding a light chain or a variable region thereof of a chimeric cytokine modified antibody or antigen binding fragment of any of the preceding embodiments.

Provided herein is an expression vector comprising the polynucleotide of any of the preceding embodiments.

Provided herein is a host cell comprising the polynucleotide or the expression vector of of any of the preceding embodiments. In some of any embodiments, the host cell of further comprises a polynucleotide or vector expressing an extracellular domain of the IL15Rα comprising the IL15Rα sushi domain. In some of any embodiments, the extracellular domain of the IL15Rα comprising the IL15Rα sushi domain comprises the sequence set forth in SEQ ID NO 2.

Provided herein is a method of producing a chimeric cytokine modified antibody or antigen binding fragment comprising culturing the host cell of any of any of the preceding embodiments under conditions for expression of the antibody or antigen binding fragment by the cell, optionally further comprising recovering of purifying the antibody or antigen binding fragment.

Provided herein is a chimeric cytokine modified antibody or antigen binding fragment produced by the method of any of the preceding embodiments.

Provided herein is a pharmaceutical composition comprising the chimeric cytokine modified antibody or antigen binding fragment of any of any of the preceding embodiments.

Provided herein is a method of treating a cancer in a subject, comprising administering a therapeutically effective amount of a chimeric cytokine modified antibody or antigen binding fragment of any of the preceding embodiments.

Provided herein is a method of treating a cancer in a subject, comprising administering a therapeutically effective amount of a pharmaceutical composition of any of the preceding embodiments.

### BRIEF DESCRIPTION OF THE DRAWINGS

FIG. 1A and FIG. 1B depict a schematic representation of the generated constructs. FIG. 1A shows the crystal structure of BLV1H12 depicting how the two β-stranded stalk protrudes from the bovine VH immunoglobulin domain and terminates in an unusual three disulfide-linked knob domain (left) and the crystal structure of the B15_IL15Rα sushi variant of BLV1H12, in which the knob region has been replaced with an IL-15 cytokine domain and further contains an IL15Rα sushi domain (right). FIG. 1B depicts the different fusion antibody constructs BLV1H12-IL-15 (B15), BLV1H12-IL-15-Rαsushi (B15_Rαsushi) and BLV1H12-IL-15-GS-Rαsushi (B15_GS_Rαsushi).

FIG. 2 shows the expression of purified B15 fusion antibody constructs expressed from HEK 293 cells BLV1H12-IL-15 (B15), BLV1H12-IL-15-Rαsushi (B15_Rαsushi) and BLV1H12-IL-15-GS-Rαsushi (B15_GS_Rαsushi) and analyzed by SDS-PAGE gel electrophoresis.

FIG. 3A and FIG. 3B depict the ability of chimeric BLV1H12-IL-15 (B15) fusion antibodies to bind to the IL2/15Rβ receptor as shown through an ELISA assay. FIG. 3A depicts the ability of the B15 antibody to bind to IL2Rα or IL15Rα receptor subunits. FIG. 3B depicts the ability of B15 antibodies to bind the IL2/15Rβ receptor subunit in the presence or absence of the IL15Rα subunit.

FIG. 4 depicts the activation of the IL2/15Rβ and γc receptor and STAT5 signaling by chimeric B15 molecules, through induction and secretion of the STAT5 inducible alkaline phosphatase (SEAP) reporter gene in HEK-Blue IL2 reporter cells.

FIG. 5 depicts the activation of the IL2/15Rβ and γc receptor and STAT5 signaling by alternative chimeric B15 molecules associated with the IL15Rαsushi domain, through induction and secretion of the STAT5 inducible alkaline phosphatase (SEAP) reporter gene in HEK-Blue IL2 reporter cells.

FIG. 6 depicts the ability of chimeric B15 molecules to expand NK-92 natural killer cells. NK-92 cells were incubated with either 2-fold serially diluted (from 1.33 nM to 0.005 nM) of IL2 or IL15 monomers (R&D Systems), or chimeric B15, chimeric variant B15_Rαsushi, or chimeric B15 variant B15_GS_Rαsushi antibodies and analyzed by MTT assay.

FIG. 7 depicts the ability of chimeric B15 antibodies compared to chimeric B15 variants B15-Rαsushi or B15-GS-Rαsushi antibodies to expand NK-92 natural killer cells, as shown through MTT assay.

FIG. 8A and FIG. 8B depict a schematic representation of the generated constructs. FIG. 8A shows the crystal structure of BLV1H12 depicting how the two β-stranded stalk protrudes from the bovine VH immunoglobulin domain and terminates in an unusual three disulfide-linked knob domain (left) and the crystal structure of the chimeric BLV1H12-IL-2 (B2) fusion antibody generated by replacing the IL15 region of the chimeric B15 antibody with IL-2 (right). FIG. 8B depicts a representation of the BLV1H12-IL-2 (B2) fusion antibody containing the IL-2 sequence in the knob domain.

FIG. 9 shows the expression of the purified fusion antibody constructs BLV1H12-IL-2 (B2) expressed from HEK 293 cells and analyzed through SDS-PAGE gel electrophoresis.

FIG. 10 depicts the ability of chimeric BLV1H12-IL-2 (B2) fusion antibodies to bind to the IL2Rα and IL15Rα, as shown through enzyme-linked immunosorbent assay (ELISA).

FIG. 11 depicts the activation of the IL2/15Rβ and γc receptor and STAT5 signaling by the chimeric B2 molecule, through induction and secretion of the STAT5 inducible alkaline phosphatase (SEAP) reporter gene in HEK-Blue IL2 reporter cells.

FIG. 12 depicts the ability of the chimeric B2 molecule to expand NK-92 natural killer cells. NK-92 cells were incubated with either 2-fold serially diluted (from 1.33 nM to 0.005 nM) of IL2 monomers (R&D Systems) or chimeric B2 antibodies and analyzed by MTT assay.

FIG. 13 depicts the ability of chimeric B15 molecules to stimulate NK cells and T cells in human PBMCs in vitro. PBMCs were incubated with B15 and B15_Rαsushi at a final concentration from 250 nM to 0.016 nM, after which PBMCs were stained with anti-CD3-FITC (SK7), anti-CD4-PE (OKT4), anti-CD8a-eFluor 450 (SKi) and anti-CD56-APC (AF12-7H3) and subsequently analyzed using Novocyte Advanteon Flow Cytometer (Agilent, Santa Clara, Calif.).

### DETAILED DESCRIPTION

Provided herein are chimeric cytokine modified antibody fusion molecules in which an IL-15 or IL-2 sequence, or a biologically active portion thereof, replaces a portion of an ultralong CDR3 region of a heavy chain of a bovine (cow) antibody or a humanized sequence thereof. In some embodiments, the ultralong CDR3 region contains an ascending stalk region, a knob region and a descending stalk region, such as present in bovine antibodies, in which all or a portion of the knob region is replaced by the cytokine sequence. In some embodiments, the cytokine sequence is IL-2 or the biologically active portion thereof, for example the IL-2 has the sequence set forth in SEQ ID NO: 165. In some embodiments, the cytokine sequence is IL-15 or the biologically active portion thereof, for example the IL-15 has the sequence set forth in SEQ ID NO: 1. Also provided herein are variant chimeric IL-15 modified antibodies that include such antibodies linked or complexed with an extracellular portion of the IL15Rα, such as the IL15Rα sushi domain (e.g. set forth in SEQ ID NO:2).

IL-15 and IL-2 are pleiotropic cytokines that play important roles in both innate and adaptive immunity. IL-15 was originally described, like IL-2, as a T cell growth factor. For example, IL-15 is involved in the generation of multiple lymphocyte subsets, including natural killer (NK), NK-T cells, and memory CD8 T cells. IL-15 is also a chemotactic for T-cells, acts on neutrophils to induce morphological cell shape changes, and stimulates IL-8 production. Both cytokines belong to the four α-helix bundle family, and their membrane receptors share two subunits (the IL-2R/IL-15Rβ and γ chains) responsible for signal transduction. IL-15 functions through the trimeric IL-15R complex, which is made up of a high affinity binding α-chain (IL-15Rα) and the common IL-2Rβ- and γ-chains. The IL-2Rβ/γ complex is an intermediate affinity receptor for both cytokines that is expressed by most NK cells and can be activated in vitro by nanomolar concentrations of IL-2 or IL-15. (Wei et al. J Immunol. 2001, 167(1) 277-282; Mortier et al. J Biol Chem. 2006, 281 (3): 1612-1619).

The IL-15Rα and IL-2Rα subunits form a sub-family of cytokine receptors containing an extracellular portion that is a so called “sushi” structural domains (one in IL-15Rα and two in IL-2Rα), at their N terminus, which are also found in complement or adhesion molecules. The IL-15Rα Sushi domain is a common motif in protein-protein interaction. Sushi domains are also known as short consensus repeats or type 1 glycoprotein motifs. They have been identified on a number of protein-binding molecules, including complement components C1r, C1s, factor H, and C2m as well as the nonimmunologic molecules factor XIII and β₂-glycoprotein. A typical Sushi domain has approximately 60 aa residues and contains four cysteines. The first cysteine forms a disulfide bond with the third cysteine, and the second cysteine forms a disulfide bridge with the fourth cysteine. The two disulfide bonds are essential to maintain the tertiary structure of the protein (Kato et al. Biochemistry. 1991, 30:11687; Bottenus et al. Biochemistry 1990, 29:11195; Ranganathan et al. Pac. Symp. Biocomput. 2000, 00:155). The high affinity receptor α (IL15Rα) is involved in increasing IL15 mediated trans signaling to the receptor β and γ subunits (IL2/15Rβ and γc).

In some embodiments, the IL-2 stimulates the proliferation, activation and, in some cases, cytotoxicity of cytotoxic T lymphocytes and natural killer (NK) cells. In some embodiments, the IL-15 stimulates the proliferation, activation and, in some cases, cytotoxicity of cytotoxic T lymphocytes and natural killer (NK) cells. IL-15 may be a better candidate drug than IL-2 because it does not cause vascular leak syndrome or stimulate regulatory T cells. Although these activities make IL-2 and IL-15 desirable for therapeutic uses, IL-2 and IL-15 are difficult to express as a stable soluble protein and have a short half-life in vitro and in vivo.

The provided embodiments address these problems. Among the provided embodiments are chimeric antibodies in which an IL-2 or IL-15 cytokine sequence or a biologically active portion thereof replaces all or a portion of the knob region of a bovine antibody or a humanized variant thereof. The provided antibodies containing an IL-15 cytokine sequence or biologically active portion thereof can further be linked or complexed with an extracellular portion of the IL15Rα, such as the IL15Rα sushi domain, to further mediate IL15 activity. It is found herein that the provided chimeric molecules, including chimeric IL2 molecules (e.g. B2) or chimeric IL15 molecules (e.g. B15) and variants thereof complexed or linked with an extracellular portion of the IL15Rα, can be expressed and purified similar to typical human antibodies, and exhibit efficient binding and activity to IL2/15Rβ and γc subunits. In particular, the provided molecules function similarly to the respective IL-2 or IL15 soluble monomer cytokine in in vitro signaling assays but can be easily produced in mammalian cells and with increased stability.

Such antibodies may be useful for the treatment or prevention of a variety of diseases, disorders, or conditions, including inflammatory diseases, disorders or conditions, autoimmune diseases, disorders or conditions, metabolic diseases, disorders or conditions, neoplastic diseases, disorders or conditions, and cancers.

The present disclosure also provides methods and materials for the preparation of the provided chimeric cytokine modified antibodies, including chimeric IL-15 modified antibodies and chimeric IL-2 modified antibodies.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the articles “a” and “an” refer to one or to more than one (i.e. to at least one) of the grammatical object of the article. By way of example, “an element” means one element or more than one element.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of 20% or ±10%, more preferably +5%, even more preferably +10%, and still more preferably +0.1% from the specified value, as such variations are appropriate to perform the disclosed methods.

An “ultralong CDR3” or an “ultralong CDR3 sequence”, used interchangeably herein, comprises a CDR3 or CDR3 sequence that is not derived from a human antibody sequence. An ultralong CDR3 may be 35 amino acids in length or longer, for example, 40 amino acids in length or longer, 45 amino acids in length or longer, 50 amino acids in length or longer, 55 amino acids in length or longer, or 60 amino acids in length or longer. Typically, the ultralong CDR3 is a heavy chain CDR3 (CDR-H3 or CDRH3). An ultralong CDR3H3 exhibits features of a CDRH3 of a ruminant (e.g., bovine) sequence. The length of the ultralong CDR3 may include a non-antibody sequence, such as a cytokine sequence, for example IL-15.

“Substantially similar,” or “substantially the same”, refers to a sufficiently high degree of similarity between two numeric values (generally one associated with an antibody disclosed herein and the other associated with a reference/comparator antibody) such that one of skill in the art would consider the difference between the two values to be of little or no biological and/or statistical significance within the context of the biological characteristic measured by said values (e.g., Kd values). The difference between said two values is preferably less than about 50%, preferably less than about 40%, preferably less than about 30%, preferably less than about 20%, preferably less than about 10% as a function of the value for the reference/comparator antibody.

“Binding affinity” generally refers to the strength of the sum total of noncovalent interactions between a single binding site of a molecule (e.g., an antibody) and its binding partner (e.g., an antigen). Unless indicated otherwise, “binding affinity” refers to intrinsic binding affinity which reflects a 1:1 interaction between members of a binding pair (e.g., antibody and antigen). The affinity of a molecule X for its partner Y can generally be represented by the dissociation constant. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure.

“Percent (%) amino acid sequence identity” with respect to a peptide or polypeptide sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MegAlign (DNASTAR) software. Those skilled in the art can determine appropriate parameters for measuring alignment, including any algorithms needed to achieve maximal alignment over the full length of the sequences being compared.

“Polypeptide,” “peptide,” “protein,” and “protein fragment” may be used interchangeably to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

“Amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refers to compounds that have the same basic chemical structure as a naturally occurring amino acid, e.g., an alpha carbon that is bound to a hydrogen, a carboxyl group, an amino group, and an R group, e.g., homoserine, norleucine, methionine sulfoxide, methionine methyl sulfonium. Such analogs can have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

“Conservatively modified variants” applies to both amino acid and nucleic acid sequences. “Amino acid variants” refers to amino acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated (e.g., naturally contiguous) sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, silent variations of a nucleic acid which encodes a polypeptide is implicit in a described sequence with respect to the expression product, but not with respect to actual probe sequences. As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” including where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles disclosed herein. Typically conservative substitutions include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

“Humanized” or “Human engineered” forms of non-human (e.g., bovine) antibodies are chimeric antibodies that contain amino acids represented in human immunoglobulin sequences, including, for example, wherein minimal sequence is derived from non-human immunoglobulin. For example, humanized or human engineered antibodies may be non-human (e.g., bovine) antibodies in which some residues are substituted by residues from analogous sites in human antibodies (see, e.g., U.S. Pat. No. 5,766,886). A humanized antibody optionally may also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1: 105-115 (1998); Harris, Biochem. Soc. Transactions 23:1035-1038 (1995); Hurle and Gross, Curr. Op. Biotech. 5:428-433 (1994).

A “variable domain” with reference to an antibody refers to a specific Ig domain of an antibody heavy or light chain that contains a sequence of amino acids that varies among different antibodies. Each light chain and each heavy chain has one variable region domain (VL, and, VH). The variable domains provide antigen specificity, and thus are responsible for antigen recognition. Each variable region contains CDRs that are part of the antigen binding site domain and framework regions (FRs).

A “constant region domain” refers to a domain in an antibody heavy or light chain that contains a sequence of amino acids that is comparatively more conserved among antibodies than the variable region domain. Each light chain has a single light chain constant region (CL) domain and each heavy chain contains one or more heavy chain constant region (CH) domains, which include, CH1, CH2, CH3 and, in some cases, CH4. Full-length IgA, IgD and IgG isotypes contain CH1, CH2 CH3 and a hinge region, while IgE and IgM contain CH1, CH2 CH3 and CH4. CH1 and CL domains extend the Fab arm of the antibody molecule, thus contributing to the interaction with antigen and rotation of the antibody arms. Antibody constant regions can serve effector functions, such as, but not limited to, clearance of antigens, pathogens and toxins to which the antibody specifically binds, e.g. through interactions with various cells, biomolecules and tissues.

The term, “corresponding to” with reference to positions of a protein, such as recitation that nucleotides or amino acid positions “correspond to” nucleotides or amino acid positions in a disclosed sequence, such as set forth in the Sequence listing, refers to nucleotides or amino acid positions identified upon alignment with the disclosed sequence based on structural sequence alignment or using a standard alignment algorithm, such as the GAP algorithm. For example, corresponding residues of a similar sequence (e.g. fragment or species variant) can be determined by alignment to a reference sequence by structural alignment methods. By aligning the sequences, one skilled in the art can identify corresponding residues, for example, using conserved and identical amino acid residues as guides.

The term “effective amount” or “therapeutically effective amount” as used herein means an amount of a pharmaceutical composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, a composition refers to any mixture of two or more products, substances, or compounds, including cells. It may be a solution, a suspension, liquid, powder, a paste, aqueous, non-aqueous or any combination thereof.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

As used herein, “disease or disorder” refers to a pathological condition in an organism resulting from cause or condition including, but not limited to, infections, acquired conditions, genetic conditions, and characterized by identifiable symptoms.

As used herein, the terms “treat,” “treating,” or “treatment” refer to ameliorating a disease or disorder, e.g., slowing or arresting or reducing the development of the disease or disorder, e.g., a root cause of the disorder or at least one of the clinical symptoms thereof.

As used herein, the term “subject” refers to an animal, including a mammal, such as a human being. The term subject and patient can be used interchangeably.

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

### II. Chimeric Cytokine Modified Antibodies

Provided herein are chimeric modified antibodies in which a cytokine sequence, such as an IL-2 sequence or a biologically active portion thereof or an IL-15 sequence or a biologically active portion thereof replaces a portion of an ultralong CDR3 region of a heavy chain of a bovine (cow) antibody or a humanized sequence thereof. The provided chimeric modified IL-15 antibodies also include such antibodies that are linked to or complexed to an extracellular portion of the IL15Rα, such as the IL15Rα sushi domain (e.g. set forth in SEQ ID NO:2).

The provided antibodies exhibit features of bovine or cow antibodies that have unique heavy chain variable region sequences containing an ultralong CDR3 sequence of up to 70 amino acids or more in length. CDR3 sequence identified in cattle include those designated as: BLV1 H12 (see, SEQ ID NO: 25), BLV5B8 (see, SEQ ID NO: 30), BLV5D3 (see, SEQ ID NO: 31) and BLV8C1 1 (see, SEQ ID NO: 32) (see, e.g., Saini, et al. (1999) Eur. Immunol. 29: 2420-2426; and Saini and Kaushik (2002) Scand. J. Immunol. 55: 140-148); BF4E9 (see, SEQ ID NO: 33) and BF1 H1 (see, SEQ ID NO: 34) (see, e.g., Saini and Kaushik (2002) Scand. J. Immunol. 55: 140-148); and F18 (see, SEQ ID NO: 35) (see, e.g., Berens, et al. (1997) Int. Immunol. 9: 189-199). Exemplary antibody variable region sequences comprising an ultralong CDR3 sequence identified in cattle include BLV1H12. In some embodiments, the BLV1H12 ultralong CDR3 sequence is encoded by the SEQ ID NO: 25. An exemplary bovine antibody includes bovine antibody BLVH12 (e.g., heavy chain variable region set forth in SEQ ID NO: 26, and light chain variable region set forth in SEQ ID NO: 27); and bovine antibody BLV5B8 (e.g., heavy chain variable region set forth in SEQ ID NO: 28, and light chain variable region set forth in SEQ ID NO: 29).

In cow antibodies, the ultralong CDR3 sequences form a structure where a subdomain with an unusual architecture is formed from a “stalk”, composed of two 12-residue, anti-parallel 3-strands (ascending and descending strands), and a 39-residue, disulfide-rich “knob” that sits atop the stalk, far from the canonical antibody paratope. The long anti-parallel β-ribbon serves as a bridge to link the knob domain with the main antibody scaffold. The unique “stalk and knob” structure of the ultralong CDR3 results in the two antiparallel β-strands, an ascending and descending stalk strand, supporting a disulfide bonded knob protruding out of the antibody surface to form a mini antigen binding domain. In some embodiments, the ultralong CDR3 antibodies comprise, in order, an ascending stalk region, a knob region, and a descending stalk region.

The unique “stalk” and knob structural features are conserved across the different bovine or cow ultralong CDR3 sequences. The ascending strand of the stalk comprises mainly hydrophobic side chains and a relatively conserved “T(T/S)VHQ” motif and variants thereof at the base, which initiates the ascending strand. This conserved T(T/S)VHQ motif and variants thereof is typically found following the first cysteine residue in variable region sequences of the various bovine or cow sequences. The conserved T(T/S)VHQ motif is connected by a variable number of residues to a motif (CPDG for BLV1H12) that forms a β-turn at the base of each knob. The stalk can be of variable length, and the descending strand of the stalk comprises alternating aromatics that form a ladder through stacking interactions, that may contribute to the stability of the long solvent-exposed, two stranded β-ribbon (Wang et al. Cell. 2013, 153 (6): 1379-1393).

The ultralong CDR3 sequences of the heavy chain of chimeric antibodies provided herein contains a stalk component that contains an ascending strand and descending strand, joined together by a knob domain that contains a cytokine sequence, such as an IL-2 sequence or a biologically active portion thereof or an IL-15 sequence or a biologically active portion thereof. In some embodiments, the provided antibodies include cytokine (e.g. IL-2 or IL-15) modified ultralong CDR3 fusions in which the antibody sequence is based on or derived from a bovine or cow sequence, or a humanized sequence thereof, that has an ultralong CDR3 in the heavy chain, but in which the ultralong CDR3 is modified to contain a non-antibody cytokine sequence compared to the ultralong CDR3 from which the antibody sequence is derived. In some embodiments, the non-antibody sequence is IL-2 or a biologically active portion thereof and the IL-2 or biologically active portion thereof may be inserted into the portion of the ultralong CDR3. In some embodiments, the non-antibody sequence is IL-15 or a biologically active portion thereof and the IL-15 or biologically active portion thereof may be inserted into the portion of the ultralong CDR3. For example, the antibody scaffold may be derived from or based on a bovine antibody sequence, or a humanized sequence thereof, but include the cytokine sequence, e.g. IL-2 sequence or biologically active portion thereof or IL-15 sequence or biologically active portion thereof, inserted into or replacing a portion of the knob domain of the ultralong CDR3 of the heavy chain of the bovine antibody sequence or the humanized sequence thereof.

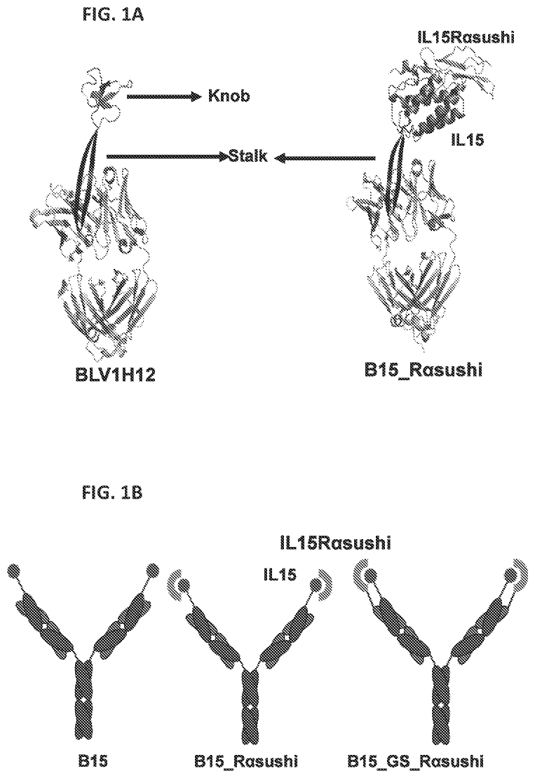

In some embodiments, the IL-15 sequence or a biologically active portion thereof is inserted into the knob region of the CDR3 sequence of the antibody, including optionally, removing a portion of CDR3 (e.g., one or more amino acids of the CDR3) or the entire CDR3 sequence (e.g., all or substantially all of the amino acids of the CDR3). In some embodiments, the IL-15 or biologically active portion thereof may be inserted into the knob domain of the ultralong CDR3 (FIG. 1A and FIG. 1B). In some embodiments, the IL-15 or biologically active portion thereof is contained between the ascending and descending stalk strands.

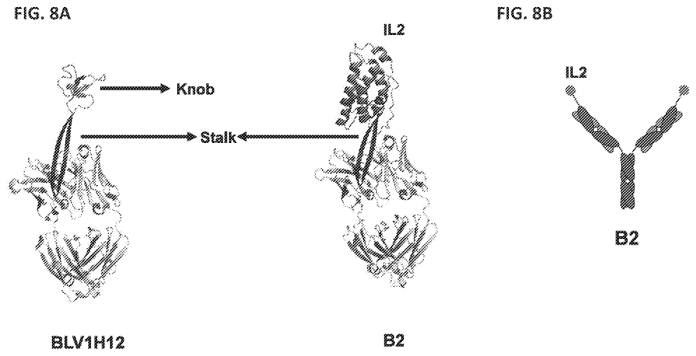

In some embodiments, the IL-2 sequence or a biologically active portion thereof is inserted into the knob region of the CDR3 sequence of the antibody, including optionally, removing a portion of CDR3 (e.g., one or more amino acids of the CDR3) or the entire CDR3 sequence (e.g., all or substantially all of the amino acids of the CDR3). In some embodiments, the IL-2 or biologically active portion thereof may be inserted into the knob domain of the ultralong CDR3 (FIG. 8A and FIG. 8B). In some embodiments, the IL-2 or biologically active portion thereof is contained between the ascending and descending stalk strands.

In some embodiments, the ultralong CDR3 may be 35 amino acids in length or more (e.g., 40 or more, 45 or more, 50 or more, 55 or more, 60 or more).

Any of the embodiments provided herein can contain any of the features as described in PCT/US2013/020910, PCT/US2014/047315 or PCT/US2013/020903, all of which are incorporated by reference in their entirety.

A. Heavy Chain Regions

In provided embodiments, the heavy chain of the provided chimeric cytokine modified antibodies is based on or derived from a framework sequence that has an ultralong CDR3, in which the cytokine sequence, e.g. IL-2 or a biologically active portion thereof or IL-15 or biologically active portion thereof, is inserted into or replaces at least a portion of the ultralong CDR3 sequence. The antibody framework may be derived from a bovine sequence such as VH-VL, a human germline sequence, or a modified human germline sequence.

In some embodiments, the heavy chain of the provided chimeric cytokine modified antibodies is based on or derived from a bovine or cow framework sequence in which the cytokine sequence, e.g. IL-2 or a biologically active portion thereof or IL-15 or biologically active portion thereof, can be inserted into or replace at least a portion of the ultralong CDR3 sequence of a bovine or cow sequence. The antibody may comprise at least a portion of a BLV1H12 antibody containing an ultralong CDR3 fusion containing the cytokine sequence. Alternatively, or additionally, the antibody comprises at least a portion of a BLV5D3, BLV8C11, BF1H1, BLV5B8 and/or F18 antibody containing an ultralong CDR3 fusion containing the cytokine sequence. In some embodiments, the IL-15 or biologically active portion thereof can be inserted into or replace at least a portion of the ultralong CDR3 of the a sequence set forth in SEQ ID NO:26 or SEQ ID NO:28.

In some embodiments, the heavy chain of the provided chimeric IL-15 modified antibodies is a based on or derived from a humanized heavy chain framework sequence that is humanized compared to a bovine or cow sequence. In some embodiments, the heavy chain of the provided chimeric cytokine modified antibodies is a based on or derived from a human heavy chain framework sequence that exhibits sequence or structural similarities to a bovine or cow sequence. In some cases, humanization can include engineering an ultralong CDR3 sequence derived from a bovine ultralong CDR3, such as any described above, into a human framework. The human framework may be of germline origin, or may be derived from non-germline (e.g. mutated or affinity matured) sequences. Genetic engineering techniques well known to those in the art, including as disclosed herein, may be used to generate a hybrid DNA sequence containing a human framework and a non-human ultralong CDR3. Unlike human antibodies which may be encoded by V region genes derived from one of seven families, bovine antibodies which produce ultralong CDR3 sequences appear to utilize a single V region family which may be considered to be most homologous to the human VH4 family. In particular embodiments where ultralong CDR3 sequences derived from cattle are to be humanized to produce an antibody comprising an ultralong CDR3, human V region sequences derived from the VH4 family may be genetically fused to a bovine-derived ultralong CDR3 sequence. Exemplary VH4 germline gene sequences in the human antibody locus include, but are not limited to, VH4-39, VH4-59*03, VH4-34*02 or VH4-34*09 human heavy chain germline sequences. In some embodiments, the human heavy chain germline sequence is a sequence set forth in any one of SEQ ID NOS: 68-71. In some embodiments, the human heavy chain germline sequence is a sequence encoded by the sequence set forth in any one of SEQ ID Nos: 169-172.

In some embodiments, the cytokine sequence, such as IL-2 or a biologically active portion thereof or IL-15 or biologically active portion thereof, can be inserted into or replace at least a portion of the ultralong CDR3 of a human germline sequence comprising the sequence set forth in SEQ ID NOs: 68-71.

In some embodiments, the provided antibodies include a fusion of a human VH4 framework sequence to a bovine-derived ultralong CDR3 into which at least a portion of the knob is replaced with IL-15 or IL-2 or a biologically active portion thereof. In some aspects, such fusions can be generated through the following steps. First, the second cysteine of a V region genetic sequence is identified along with the nucleotide sequence encoding the second cysteine. Generally, the second cysteine marks the boundary of the framework and CDR3 two residues upstream (N-terminal) of the CDR3. Second, the second cysteine in a bovine-derived V region sequence is identified which similarly marks 2 residues upstream (N-terminal) of the CDR3. Third, the genetic material encoding the human V region is combined with the genetic sequence encoding the ultralong CDR3. Thus, a genetic fusion may be made, wherein the ultralong CDR3 sequence is placed in frame of the human V region sequence. Preferably a humanized antibody comprising an ultralong CDR3 is as near to human in amino acid composition as possible. Optionally, a J region sequence may be mutated from bovine-derived sequence to a human sequence. Also optionally, a humanized heavy chain may be paired with a human light chain.

In some embodiments, the antibody or binding fragment thereof comprises a heavy chain variable region comprising a sequence of the formula V1-X-V2, wherein the V1 region of the heavy chain comprises a heavy chain sequence portion containing three framework regions (e.g. FR-1, FR-2 and FR-3) separating two CDR regions (CDR1 and CDR3), wherein the X comprises an ultralong CDR3 sequence, which can include an IL-2 sequence or a biologically active portion thereof or an IL-15 sequence or a biologically active portion thereof, and wherein the V2 comprises a portion of the heavy chain including FR-4.

In some embodiments, the V1 region comprises the formula FR1-CDR1-FR2-CDR2-FR3. In some embodiments, the V1 region comprises an amino acid sequence selected from the group consisting of: (i) bovine heavy chain regions comprising amino acids of SEQ ID NO: 26 (encoded by the nucleotide of SEQ ID NO:5), or (i) a humanized heavy chain regions comprising human germline variable regions comprising SEQ ID NOS: 12-19.

In some embodiments, X comprises the ultralong CDR3 sequence, which can include an IL-15 sequence or a biologically active portion thereof (e.g., a human IL-15 sequence or a biologically active portion thereof). In some embodiments, the IL-15 sequence comprises the amino acid sequence set forth in SEQ ID NO:1 or a sequence of amino acids that exhibits at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, at least at or about 99% sequence identity to the amino acid sequence set forth in SEQ ID NO:1. In some embodiments, the IL-15 sequence comprises the amino acid sequence found in SEQ ID NO: 1.

In some embodiments, the IL-15 sequence exhibits activity to stimulate the proliferation, activation or cytotoxicity of cytotoxic T lymphocytes and natural killer (NK) cells, such as in an in vitro assay or in vivo. In some embodiments, the IL-15 sequence exhibits binding to IL2/15Rβ and/or ye subunits, such as in an in vitro binding assay. In some embodiments, the activity or binding is similar to or retained compared to a recombinant IL-15 monomer.

In some embodiments, the IL-15 sequence or biologically active portion is inserted into the knob of the ultralong CDR3 between the ascending and descending stalk regions. The IL-15 sequence may be positioned between the stalk regions, in which the IL-15 sequence is linked directly or indirectly to each of the stalk regions. In some embodiments, the linkage to one or both of the stalk sequences is indirect via a linker. The linker can comprise an amino acid sequence of (GGGGS), wherein n=1 to 5. Alternatively, the linker comprises an amino acids sequence of (GSG)n, GGGSGGGGS or GGGGSGGGS. In some cases, the linker has the sequence GGS or GSG.

In some embodiments, X comprises the ultralong CDR3 sequence, which can include an IL-2 sequence or a biologically active portion thereof (e.g., a human IL-2 sequence or a biologically active portion thereof). In some embodiments, the IL-2 sequence comprises the amino acid sequence set forth in SEQ ID NO: 165 or a sequence of amino acids that exhibits at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, at least at or about 99% sequence identity to the amino acid sequence set forth in SEQ ID NO:165. In some embodiments, the IL-2 sequence comprises the amino acid sequence found in SEQ ID NO: 165.

In some embodiments, the IL-2 sequence exhibits activity to stimulate the proliferation, activation or cytotoxicity of cytotoxic T lymphocytes and natural killer (NK) cells, such as in an in vitro assay or in vivo. In some embodiments, the IL-2 sequence exhibits binding to IL2/15Rβ and/or γc subunits, such as in an in vitro binding assay. In some embodiments, the activity or binding is similar to or retained compared to a recombinant IL-2 monomer.

In some embodiments, the IL-2 sequence or biologically active portion is inserted into the knob of the ultralong CDR3 between the ascending and descending stalk regions. The IL-2 sequence may be positioned between the stalk regions, in which the IL-2 sequence is linked directly or indirectly to each of the stalk regions. In some embodiments, the linkage to one or both of the stalk sequences is indirect via a linker. The linker can comprise an amino acid sequence of (GGGGS), wherein n=1 to 5. Alternatively, the linker comprises an amino acids sequence of (GSG)n, GGGSGGGGS or GGGGSGGGS. In some cases, the linker has the sequence GGS or GSG.

The ultralong CDR3 may comprise at least a portion of a knob domain of a CDR3, at least a portion of a stalk domain of a CDR3, or a combination thereof. The portion of the knob domain of the CDR3 may comprise one or more conserved motifs derived from the knob domain of the ultralong CDR3. The stalk domain of the CDR3 may comprise one or more conserved motifs derived from the stalk domain of the ultralong CDR3.

In aspects of each or any of the above or below mentioned embodiments, the ultralong CDR3 is 35 amino acids in length or longer, 40 amino acids in length or longer, 45 amino acids in length or longer, 50 amino acids in length or longer, 55 amino acids in length or longer, or 60 amino acids in length or longer. In some embodiments of each or any of the above or below mentioned embodiments, the ultralong CDR3 is 35 amino acids in length or longer

In some embodiments, the X portion of a heavy chain that includes the ultralong CDR3 includes the motif X¹X²X³X⁴X⁵-[cytokine sequence]-(X^{a}X^{b})z motif. In some embodiments, the ultralong CDR3 is 45 amino acids in length or longer. In some embodiments one or more additional amino acids may be present between the X¹X²X³X⁴X⁵ motif and the cytokine sequence and/or between the (X^{a}X^{b})z motif and the cytokine sequence.

In some embodiments, the X¹X²X³X⁴X⁵ motif is all or a portion of the ascending stalk strand. In some embodiments, the X¹X²X³X⁴X⁵ motif on the ascending stalk strand comprises a sequence selected from TTVHQ (SEQ ID NO: 36), TSVHQ (SEQ ID NO: 37) or any one of SEQ ID NOs: 38-67. In some embodiments, the ascending stalk strand comprises a sequence selected from SEQ ID NOs: 72-75 or SEQ ID NO:158. In some embodiments, the ultralong CDR3 comprises an ascending stalk region encoded by SEQ ID NO: 9, SEQ ID NO: 81-121 or SEQ ID NO:157. In some embodiments, the motif includes an N-terminal cysteine (Cys or C) residue, such as set forth a CX¹X²X³X⁴X⁵. For example, in some cases, an ascending stalk region encoded by any of SEQ ID NOs: 36-67, 72-75 or SEQ ID NO:158 may additionally contain an N-terminal Cys residue. Such an exemplary ascending stalk region is set forth in SEQ ID NO:159.

In some embodiments, the (X^{a}X^{b})z motif is a portion of the descending stalk strand, wherein X^{a} is any amino acid residue, X^{b} is an aromatic amino acid selected from the group consisting of: tyrosine (Y), phenylalanine (F), tryptophan (W), and histidine (H), and wherein z is 1-4. In some embodiments, the descending stalk strand comprises alternating aromatics with the formula YXYXYX where is X is any amino acid. In some embodiments, the descending stalk strand comprises a sequence contained in SEQ ID NO: 76-80 or SEQ ID NO:161. In some embodiments, the ultralong CDR3 comprises a descending stalk region encoded by SEQ ID NO: 10, SEQ ID NO: 122-149 or SEQ ID NO:160.

In some embodiments, the ultralong CDR3 comprises, in order an ascending stalk region having an amino acid sequence encoded by SEQ ID NO:9, an IL15 cytokine sequence set forth by SEQ ID NO: 1, and a descending stalk region having an amino acid sequence encoded by SEQ ID NO: 10. In some embodiments, the ultralong CDR3 comprises, in order an ascending stalk region having an amino acid sequence encoded by SEQ ID NO:157, an IL15 cytokine sequence set forth by SEQ ID NO: 1, and a descending stalk region having an amino acid sequence encoded by SEQ ID NO: 160.

In some embodiments, the ultralong CDR3 comprises, in order an ascending stalk region having an amino acid sequence encoded by SEQ ID NO:9, an IL2 cytokine sequence set forth by SEQ ID NO: 165, and a descending stalk region having an amino acid sequence encoded by SEQ ID NO: 10. In some embodiments, the ultralong CDR3 comprises, in order an ascending stalk region having an amino acid sequence encoded by SEQ ID NO:157, an IL2 cytokine sequence set forth by SEQ ID NO: 165, and a descending stalk region having an amino acid sequence encoded by SEQ ID NO: 160.

In some embodiments, the V2 region of the heavy chain comprises an amino acid sequence selected from the group consisting of (i) WGHGTAVTVSS (SEQ ID NO: 20), (ii) WGKGTTVTVSS (SEQ ID NO: 21), (iii) WGKGTTVTVSS (SEQ ID NO: 22), (iv) WGRGTLVTVSS (SEQ ID NO: 23), (v) WGKGTTVTVSS (SEQ ID NO: 24), and (vi) WGQGLLVTVSS (SEQ ID NO: 11).

In particular embodiments, a chimeric IL-15 modified antibody or antigen-binding fragment provided herein contains a variable heavy chain sequence encoded by the sequence of nucleotides set forth in SEQ ID NO:7 or a sequence of nucleotides that exhibits at least at or about 85%, a at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, at least at or about 99% sequence identity to the nucleotide sequence set forth in SEQ ID NO:7, in which is contained a modified ultralong CDR3 containing an IL-15 sequence. In some embodiments, the chimeric IL-15 modified antibody or antigen-binding fragment provided herein comprises a variable heavy chain sequence encoded by the sequence of nucleotides set forth in SEQ ID NO:7. In some embodiments, the chimeric IL-15 modified antibody or antigen-binding fragment provided herein consists of or consists essentially of a variable heavy chain sequence encoded by the sequence of nucleotides set forth in SEQ ID NO:7.

In some embodiments, the heavy chain includes a variable heavy chain as described that is joined to a human constant region. In some embodiments, the human constant region includes the CH1-CH2-CH3 constant domains. In some embodiments, the human constant region is of human IgG1.

B. Light Chain Regions

In some embodiments, the antibody or antigen binding fragment further comprises a light chain variable region. In some embodiments, a chimeric cytokine modified antibody variable chain is based on a bovine sequence and is paired with a variable light chain of a bovine antibody. In another embodiment, the present disclosure provides pairing of a humanized ultralong CDR3 heavy chain with a bovine light chain. In particular embodiments, the light chain is a lambda light chain.

In some embodiments, the variable light chain is a variable light chain of a bovine antibody, such as a variable light chain of BLVH12, BLV5D3, BLV8C11, BF1H1, BLV5B8 and/or F18. In some embodiments, the light chain variable region may comprise a sequence based or derived from the polypeptide sequence of SEQ ID NO: 27 or 29. In some embodiments, the light chain polypeptide sequence is encoded by a DNA sequence based on or derived from the DNA sequence of SEQ ID NO:8. In some embodiments, the light chain polypeptide sequence is encoded by a DNA sequence based on or derived from the DNA sequence of SEQ ID NO:168.

In some embodiments, the light chain includes a variable light chain of a bovine antibody that is joined to a human lambda light chain constant region (e.g. set forth in SEQ ID NO:155). In some embodiments, a portion of the BLV1H12 light chain variable region (e.g. set forth in SEQ ID NO:8 or SEQ ID NO: 168) is joined with the human lambda light chain constant region.

In some embodiments, the light chain is a humanized light chain or is a human light chain. In embodiments, the present disclosure provides pairing of a humanized heavy chain comprising an ultralong CDR3 with a human light chain. In some embodiments, the light chain is homologous to a bovine light chain known to pair with a bovine ultralong CDR3 heavy chain. Several human VL sequences can be used to paired with the sequences above, including VL1-47, VL1-40, VL1-51, VL2-18, which are homologous to the lambda region derived from Bos Taurus. In some embodiments, the light chain variable region is a sequence set forth in any one of SEQ ID NOS: 156 or 173-176. In some embodiments, the light chain variable sequence is a sequence encoded by the sequence set forth in any one of SEQ ID Nos: 177-180. In some embodiments, the light chain variable region comprises a variable region of the VL1-51 germline sequence set forth in SEQ ID NO: 156.

In some embodiments, the light chain variable region is a human germline light chain sequence, such as any described above, that contains one or more amino acid modifications. Such modifications may include the substitution of certain amino acid residues in the human light chain to those residues at corresponding positions in a bovine light chain sequence. The modified light chains may improve the yield of the antibody comprising the ultralong CDR3 and/or increase its binding specificity. In some embodiments, the modifications include one or more of amino acid replacements S2A, T5N, P8S, A12G, A13S, and P14L based on Kabat numbering. In some embodiments, the modifications include amino acid replacements S2A, T5N, P8S, A12G, A13S, and P14L based on Kabat numbering. In some embodiments, the modifications are in the CDR1 and include amino acid replacements I29V and N32G. In some embodiments, the modifications are in the CDR2 and include substitution of DNN to GDT. In some embodiments, the modifications are n CDR2 and include a substitution DNNKRP to GDTSRA. In some embodiments, the modifications include a combination of any of the forgoing. For example, provided modifications of a human germline light chain sequence include amino acid replacements S2A, T5N, P8S, A12G, A13S, and P14L based on Kabat numbering and substitution of DNN to GDT in CDR2.

In some embodiments, the light chain includes a humanized variable light chain as described that is joined to a human lambda light chain constant region (e.g. set forth in SEQ ID NO:155. In some embodiments, a portion of the light chain variable region, such as a modified human germline light chan, is joined with the human lambda light chain constant region.

C. IL-15Rα Sushi Domain

In some embodiments, the chimeric interleukin 15 antibody molecules provided herein can further be linked or complexed with all or a portion of the IL-15 high affinity receptor α (IL15Rα), such as a portion containing an extracellular domain of the IL15Rα, such as the IL15Rα sushi domain. In some embodiments, the IL-15 cytokine sequence is linked to all or a portion of the IL-15 high affinity receptor α (IL15Rα). In some embodiments, the IL15Rα is expressed to increase trans signaling to the receptor β and γ subunits (IL2/15Rβ and γc). IL-15 high affinity receptor comprises the IL15Rα sushi domain. In some embodiments, the IL15Rα sushi domain comprises the sequence set forth in SEQ ID NO: 2.

In some embodiments, provided herein is a chimeric IL-15 modified antibody or antigen-binding fragment in which the heavy chain or variable sequence thereof includes an IL-15 sequence that replaces all or a portion of the knob of an ultralong CDR3 (e.g. is inserted into the knob region between the ascending and descending stalk) that is complexed with an extracellular domain of the IL15Rα, such as the IL15Rα sushi domain. In some embodiments, the chimeric IL-15 modified antibody or antigen-binding fragment is complexed with an IL15Rα sushi domain set forth in SEQ ID NO: 15. Such antibody molecules can be generated by co-expressing the IL15Rα extracellular domain, e.g. sushi domain, such as set forth in SEQ ID NO:2, with the heavy chain regions and the light chain regions in a host cell.

In some embodiments, provided herein is a chimeric IL-15 modified antibody or antigen-binding fragment containing a heavy chain or variable sequence thereof in which an IL-15 sequence replaces all or a portion of the knob of an ultralong CDR3 (e.g. is inserted into the knob region between the ascending and descending stalk), and a light chain or variable sequence thereof that is linked to an extracellular domain of the IL15Rα, such as the IL15Rα sushi domain. In some embodiments, the chimeric IL-15 modified antibody or antigen-binding fragment is linked to an IL15Rα sushi domain set forth in SEQ ID NO:2. The linkage between the extracellular domain of the IL15Rα (e.g. IL15Rα sushi domain, such as set forth in SEQ ID NO:2) is via a peptide linker. In some embodiments, the linker is a flexible linker such as a glycine linker or a glycine-serine (GS) linker. In some embodiments, the peptide linker is a GS linker. Exemplary GS linkers include, but are not limited to, any of the sequences set forth in SEQ ID NOs: 150-154 or encoded by the nucleotide sequences set forth in SEQ ID NO: 163 or SEQ ID NO:164. In some embodiments, the linker is GS.

In some embodiments, a chimeric IL-15 modified antibody or antigen-binding fragment provided herein contains a heavy chain or variable sequence thereof in which an IL-15 sequence replaces all or a portion of the knob of an ultralong CDR3 (e.g. is inserted into the knob region between the ascending and descending stalk), and a light chain or variable sequence thereof comprising the sequence of amino acids encoded by SEQ ID NO:3.

D. Vectors, Host Cells and Recombinant Methods

For recombinant production of an antibody or fragment thereof as disclosed herein, the nucleic acid encoding it is isolated and inserted into a replicable vector for further cloning (amplification of the DNA) or for expression. DNA encoding the antibody is readily isolated and sequenced using conventional procedures (e.g., by using oligonucleotide probes that are capable of binding specifically to genes encoding the heavy and light chains of the antibody). In an exemplary embodiment, nucleic acid encoding an antibody comprising an ultralong CDR3, a variable region comprising an ultralong CDR3, or an ultralong CDR3, is isolated and inserted into a replicable vector for further cloning (amplification of the DNA) or for expression. Many vectors are available. The choice of vector depends in part on the host cell to be used. Generally, preferred host cells are of either prokaryotic or eukaryotic (generally mammalian) origin. It will be appreciated that constant regions of any isotype can be used for this purpose, including IgG, IgM, IgA, IgD, and IgE constant regions, and that such constant regions can be obtained from any human or animal species.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase and dihydrofolate reductase. Alternatively, high yield expression systems not involving gene amplification are also suitable, such as using a baculovirus vector in insect cells, with a nucleic acid sequence encoding a partially human ultralong CDR3 antibody chain under the direction of the polyhedrin promoter or other strong baculovirus promoters.

Polynucleotide sequences encoding polypeptide components of the antibodies disclosed herein can be obtained using standard recombinant techniques. In some embodiments, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present disclosure. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence. Additionally, V regions comprising an ultralong CDR3 may optionally be fused to a C-region to produce an antibody comprising constant regions.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. pBR322 contains genes encoding ampicillin (Amp) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins. Examples of pBR322 derivatives used for expression of particular antibodies have been described (see, e.g., U.S. Pat. No. 5,648,237).

In addition, phage vectors containing replicon and control sequences that are compatible with the host microorganism can be used as transforming vectors in connection with these hosts. For example, bacteriophage such as λGEM™-11 may be utilized in making a recombinant vector which can be used to transform susceptible host cells such as E. coli LE392.

The expression vectors disclosed herein may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g., the presence or absence of a nutrient or a change in temperature.

A large number of promoters recognized by a variety of potential host cells are well known. The selected promoter can be operably linked to cistron DNA encoding the light or heavy chain by removing the promoter from the source DNA via restriction enzyme digestion and inserting the isolated promoter sequence into the vector disclosed herein. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include: an ara B promoter, a PhoA promoter, β-galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleotide sequences have been published, thereby enabling a skilled worker operably to ligate them to cistrons encoding the target light and heavy chains (e.g., Siebenlist et al. (1980) Cell 20: 269) using linkers or adaptors to supply any required restriction sites.

Suitable bacterial promoters are well known in the art and fully described in scientific literature such as Sambrook and Russell, supra, and Ausubel et al, supra. Bacterial expression systems for expressing antibody chains of the recombinant catalytic polypeptide are available in, e.g., E. coli, Bacillus sp., and Salmonella (Palva et al., Gene, 22:229-235 (1983); Mosbach et al., Nature, 302:543-545 (1983)).

In one aspect disclosed herein, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence should be one that is recognized and processed (e.g., cleaved by a signal peptidase) by the host cell. For prokaryotic host cells that do not recognize and process the signal sequences native to the heterologous polypeptides, the signal sequence is substituted by a prokaryotic signal sequence selected, for example PelB, OmpA, alkaline phosphatase, penicillinase, Ipp, or heat-stable enterotoxin II (STII) leaders, LamB, PhoE, and MBP. In one embodiment disclosed herein, the signal sequences used in both cistrons of the expression system are STII signal sequences or variants thereof.

In another aspect, the production of the immunoglobulins according to the disclosure can occur in the cytoplasm of the host cell, and therefore does not require the presence of secretion signal sequences within each cistron. In that regard, immunoglobulin light and heavy chains are expressed, folded and assembled to form functional immunoglobulins within the cytoplasm. Certain host strains (e.g., the E. coli trxB-strains) provide cytoplasm conditions that are favorable for disulfide bond formation, thereby permitting proper folding and assembly of expressed protein subunits (see e.g., Proba and Pluckthun Gene, 159:203 (1995)).

Suitable host cells for cloning or expression of antibody-encoding vectors include prokaryotic or eukaryotic cells described herein. In one embodiment, the host cell is eukaryotic, e.g. a Chinese Hamster Ovary (CHO) cell, Human Embryonic Kidney (HEK) cell or lymphoid cell (e.g., YO, NSO, Sp20 cell). For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified. In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gemgross, Nat. Biotech. 22: 1409-1414 (2004), and Li et al., Nat. Biotech. 24:210-215 (2006). Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well known plasmids such as pBR322, pBR325, pACYC177, or pKN410 are used to supply the replicon. Typically the host cell should secrete minimal amounts of proteolytic enzymes, and additional protease inhibitors may desirably be incorporated in the cell culture.

Plant cell cultures can also be utilized as hosts. See, e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., Gen V1I′0l. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (V ERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TR1 cells, as described, e.g., in Mather et al., Annals NI'. Acad. Sci. 383:44-68 (1982); MRC 5 cells; and FS4 cells. Other useful mammalian host cell lines include Chinese hamster ovary (CHO) cells, including DHFR′ CHO cells (Urlaub et al., Proc. Natl. Acad. Sci. USA 77:4216 (1980)); and myeloma cell lines such as YO, NSO and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.].), pp. 255-268 (2003).

In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody.

Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

The expressed polypeptides of the present disclosure are secreted into and recovered from the periplasm of the host cells or transported into the culture media. Protein recovery from the periplasm typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins that are transported into the culture media may be isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

Antibody production may be conducted in large quantity by a fermentation process. Various large-scale fed-batch fermentation procedures are available for production of recombinant proteins. Large-scale fermentations have at least 1000 liters of capacity, preferably about 1,000 to 100,000 liters of capacity. These fermentors use agitator impellers to distribute oxygen and nutrients, especially glucose (a preferred carbon/energy source). Small scale fermentation refers generally to fermentation in a fermentor that is no more than approximately 100 liters in volumetric capacity, and can range from about 1 liter to about 100 liters.

In a fermentation process, induction of protein expression is typically initiated after the cells have been grown under suitable conditions to a desired density, e.g., an OD550 of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To improve the production yield and quality of the polypeptides disclosed herein, various fermentation conditions can be modified. For example, to improve the proper assembly and folding of the secreted antibody polypeptides, additional vectors overexpressing chaperone proteins, such as Dsb proteins (DsbA, DsbB, DsbC, DsbD and or DsbG) or FkpA (a peptidylprolyl cis,trans-isomerase with chaperone activity) may be used to co-transform the host prokaryotic cells. The chaperone proteins have been demonstrated to facilitate the proper folding and solubility of heterologous proteins produced in bacterial host cells. (see e.g., Chen et al. (1999) J Bio Chem 274:19601-19605; U.S. Pat. Nos. 6,083,715; 6,027,888; Bothmann and Pluckthun (2000) J. Biol. Chem. 275:17100-17105; Ramm and Pluckthun (2000) J. Biol. Chem. 275:17106-17113; Arie et al. (2001) Mol. Microbiol. 39:199-210).

To minimize proteolysis of expressed heterologous proteins (especially those that are proteolytically sensitive), certain host strains deficient for proteolytic enzymes can be used for the present disclosure. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available (see, e.g., Joly et al. (1998), supra; U.S. Pat. Nos. 5,264,365; 5,508,192; Hara et al., Microbial Drug Resistance, 2:63-72 (1996)).

E. coli strains deficient for proteolytic enzymes and transformed with plasmids overexpressing one or more chaperone proteins may be used as host cells in the expression systems disclosed herein.

Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

In one aspect, Protein A immobilized on a solid phase is used for immunoaffinity purification of the full length antibody products disclosed herein. Protein A is a 41 kD cell wall protein from Staphylococcus aureus which binds with a high affinity to the Fc region of antibodies (see, e.g., Lindmark et al (1983) J. Immunol. Meth. 62:1-13). The solid phase to which Protein A is immobilized is preferably a column comprising a glass or silica surface, more preferably a controlled pore glass column or a silicic acid column. In some applications, the column has been coated with a reagent, such as glycerol, in an attempt to prevent nonspecific adherence of contaminants.

As the first step of purification, the preparation derived from the cell culture as described above is applied onto the Protein A immobilized solid phase to allow specific binding of the antibody of interest to Protein A. The solid phase is then washed to remove contaminants non-specifically bound to the solid phase. Finally the antibody of interest is recovered from the solid phase by elution.

### III. Pharmaceutical Compositions

Antibodies or antigen binding fragments comprising an ultralong CDR3, nucleic acids, or vectors disclosed herein can be formulated in compositions, especially pharmaceutical compositions. Such compositions with antibodies comprising an ultralong CDR3 comprise a therapeutically or prophylactically effective amount of antibodies comprising an ultralong CDR3, antibody fragment, nucleic acid, or vector disclosed herein in admixture with a suitable carrier, e.g., a pharmaceutically acceptable agent. Typically, antibodies comprising an ultralong CDR3, antibody fragments, nucleic acids, or vectors disclosed herein are sufficiently purified for administration before formulation in a pharmaceutical composition.

Pharmaceutically acceptable agents for use in the present pharmaceutical compositions include carriers, excipients, diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials, and surfactants.

Neutral buffered saline or saline mixed with serum albumin are exemplary appropriate carriers. The pharmaceutical compositions may include antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics, or polyethylene glycol (PEG). Also by way of example, suitable tonicity enhancing agents include alkali metal halides (preferably sodium or potassium chloride), mannitol, sorbitol, and the like. Suitable preservatives include benzalkonium chloride, thimerosal, phenethyl alcohol, methylparaben, propylparaben, chlorhexidine, sorbic acid and the like. Hydrogen peroxide also may be used as preservative. Suitable cosolvents include glycerin, propylene glycol, and PEG. Suitable complexing agents include caffeine, polyvinylpyrrolidone, beta-cyclodextrin or hydroxy-propyl-beta-cyclodextrin. Suitable surfactants or wetting agents include sorbitan esters, polysorbates such as polysorbate 80, tromethamine, lecithin, cholesterol, tyloxapal, and the like. The buffers may be conventional buffers such as acetate, borate, citrate, phosphate, bicarbonate, or Tris-HCl. Acetate buffer may be about pH 4-5.5, and Tris buffer can be about pH 7-8.5. Additional pharmaceutical agents are set forth in Remington's Pharmaceutical Sciences, 18th Edition, A. R. Gennaro, ed., Mack Publishing Company, 1990.

The composition may be in liquid form or in a lyophilized or freeze-dried form and may include one or more lyoprotectants, excipients, surfactants, high molecular weight structural additives and/or bulking agents (see, for example, U.S. Pat. Nos. 6,685,940, 6,566,329, and 6,372,716). In one embodiment, a lyoprotectant is included, which is a non-reducing sugar such as sucrose, lactose or trehalose. The amount of lyoprotectant generally included is such that, upon reconstitution, the resulting formulation will be isotonic, although hypertonic or slightly hypotonic formulations also may be suitable. In addition, the amount of lyoprotectant should be sufficient to prevent an unacceptable amount of degradation and/or aggregation of the protein upon lyophilization. Exemplary lyoprotectant concentrations for sugars (e.g., sucrose, lactose, trehalose) in the pre-lyophilized formulation are from about 10 mM to about 400 mM. In another embodiment, a surfactant is included, such as for example, nonionic surfactants and ionic surfactants such as polysorbates (e.g., polysorbate 20, polysorbate 80); poloxamers (e.g., poloxamer 188); poly(ethylene glycol) phenyl ethers (e.g., Triton); sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl ofeyl-taurate; and the MONAQUAT™, series (Mona Industries, Inc., Paterson, N.J.), polyethyl glycol, polypropyl glycol, and copolymers of ethylene and propylene glycol (e.g., Pluronics, PF68 etc). Exemplary amounts of surfactant that may be present in the pre-lyophilized formulation are from about 0.001-0.5%. High molecular weight structural additives (e.g., fillers, binders) may include for example, acacia, albumin, alginic acid, calcium phosphate (dibasic), cellulose, carboxymethylcellulose, carboxymethylcellulose sodium, hydroxyethylcellulose, hydroxypropylcellulose, hydroxypropylmethylcellulose, microcrystalline cellulose, dextran, dextrin, dextrates, sucrose, tylose, pregelatinized starch, calcium sulfate, amylose, glycine, bentonite, maltose, sorbitol, ethylcellulose, disodium hydrogen phosphate, disodium phosphate, disodium pyrosulfite, polyvinyl alcohol, gelatin, glucose, guar gum, liquid glucose, compressible sugar, magnesium aluminum silicate, maltodextrin, polyethylene oxide, polymethacrylates, povidone, sodium alginate, tragacanth microcrystalline cellulose, starch, and zein. Exemplary concentrations of high molecular weight structural additives are from 0.1% to 10% by weight. In other embodiments, a bulking agent (e.g., mannitol, glycine) may be included.

Compositions may be suitable for parenteral administration. Exemplary compositions are suitable for injection or infusion into an animal by any route available to the skilled worker, such as intraarticular, subcutaneous, intravenous, intramuscular, intraperitoneal, intracerebral (intraparenchymal), intracerebroventricular, intramuscular, intraocular, intraarterial, or intralesional routes. A parenteral formulation typically will be a sterile, pyrogen-free, isotonic aqueous solution, optionally containing pharmaceutically acceptable preservatives.

Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringers' dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present, such as, for example, anti-microbials, antioxidants, chelating agents, inert gases and the like. See generally, Remington's Pharmaceutical Science, 16th Ed., Mack Eds., 1980.

Pharmaceutical compositions described herein may be formulated for controlled or sustained delivery in a manner that provides local concentration of the product (e.g., bolus, depot effect) and/or increased stability or half-life in a particular local environment. The compositions can include the formulation of antibodies comprising an ultralong CDR3, antibody fragments, nucleic acids, or vectors disclosed herein with particulate preparations of polymeric compounds such as polylactic acid, polyglycolic acid, etc., as well as agents such as a biodegradable matrix, injectable microspheres, microcapsular particles, microcapsules, bioerodible particles beads, liposomes, and implantable delivery devices that provide for the controlled or sustained release of the active agent which then can be delivered as a depot injection. Techniques for formulating such sustained- or controlled-delivery means are known and a variety of polymers have been developed and used for the controlled release and delivery of drugs. Such polymers are typically biodegradable and biocompatible. Polymer hydrogels, including those formed by complexation of enantiomeric polymer or polypeptide segments, and hydrogels with temperature or pH sensitive properties, may be desirable for providing drug depot effect because of the mild and aqueous conditions involved in trapping bioactive protein agents (e.g., antibodies comprising an ultralong CDR3). See, for example, the description of controlled release porous polymeric microparticles for the delivery of pharmaceutical compositions in WO 93/15722.

Suitable materials for this purpose include polylactides (see, e.g., U.S. Pat. No. 3,773,919), polymers of poly-(a-hydroxycarboxylic acids), such as poly-D-(−)-3-hydroxybutyric acid (EP 133,988A), copolymers of L-glutamic acid and gamma ethyl-L-glutamate (Sidman et al., Biopolymers, 22: 547-556 (1983)), poly(2-hydroxyethyl-methacrylate) (Langer et al., J. Biomed. Mater. Res., 15: 167-277 (1981), and Langer, Chem. Tech., 12: 98-105 (1982)), ethylene vinyl acetate, or poly-D(−)-3-hydroxybutyric acid. Other biodegradable polymers include poly(lactones), poly(acetals), poly(orthoesters), and poly(orthocarbonates). Sustained-release compositions also may include liposomes, which can be prepared by any of several methods known in the art (see, e.g., Eppstein et al., Proc. Natl. Acad. Sci. USA, 82: 3688-92 (1985)). The carrier itself, or its degradation products, should be nontoxic in the target tissue and should not further aggravate the condition. This can be determined by routine screening in animal models of the target disorder or, if such models are unavailable, in normal animals.

Microencapsulation of recombinant proteins for sustained release has been performed successfully with human growth hormone (rhGH), interferon-(rhIFN-), interleukin-2, and MN rgp120. Johnson et al., Nat. Med., 2:795-799 (1996); Yasuda, Biomed. Ther., 27:1221-1223 (1993); Hora et al., Bio/Technology. 8:755-758 (1990); Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp. 439-462; WO 97/03692, WO 96/40072, WO 96/07399; and U.S. Pat. No. 5,654,010. The sustained-release formulations of these proteins were developed using poly-lactic-coglycolic acid (PLGA) polymer due to its biocompatibility and wide range of biodegradable properties. The degradation products of PLGA, lactic and glycolic acids can be cleared quickly within the human body. Moreover, the degradability of this polymer can be depending on its molecular weight and composition. Lewis, “Controlled release of bioactive agents from lactide/glycolide polymer,” in: M. Chasin and R. Langer (Eds.), Biodegradable Polymers as Drug Delivery Systems (Marcel Dekker: New York, 1990), pp. 1-41. Additional examples of sustained release compositions include, for example, EP 58,481A, U.S. Pat. No. 3,887,699, EP 158,277A, Canadian Patent No. 1176565, U. Sidman et al., Biopolymers 22, 547 [1983], R. Langer et al., Chem. Tech. 12, 98 [1982], Sinha et al., J. Control. Release 90, 261 [2003], Zhu et al., Nat. Biotechnol. 18, 24 [2000], and Dai et al., Colloids Surf B Biointerfaces 41, 117 [2005].

Bioadhesive polymers are also contemplated for use in or with compositions of the present disclosure. Bioadhesives are synthetic and naturally occurring materials able to adhere to biological substrates for extended time periods. For example, Carbopol and polycarbophil are both synthetic cross-linked derivatives of poly(acrylic acid). Bioadhesive delivery systems based on naturally occurring substances include for example hyaluronic acid, also known as hyaluronan. Hyaluronic acid is a naturally occurring mucopolysaccharide consisting of residues of D-glucuronic and N-acetyl-D-glucosamine. Hyaluronic acid is found in the extracellular tissue matrix of vertebrates, including in connective tissues, as well as in synovial fluid and in the vitreous and aqueous humor of the eye. Esterified derivatives of hyaluronic acid have been used to produce microspheres for use in delivery that are biocompatible and biodegradable (see, for example, Cortivo et al., Biomaterials (1991) 12:727-730; EP 517,565; WO 96/29998; Illum et al., J. Controlled Rel. (1994) 29:133-141). Exemplary hyaluronic acid containing compositions of the present disclosure comprise a hyaluronic acid ester polymer in an amount of approximately 0.1% to about 40% (w/w) of an antibody comprising an ultralong CDR3 to hyaluronic acid polymer.

Both biodegradable and non-biodegradable polymeric matrices may be used to deliver compositions of the present disclosure, and such polymeric matrices may comprise natural or synthetic polymers. Biodegradable matrices are preferred. The period of time over which release occurs is based on selection of the polymer. Typically, release over a period ranging from between a few hours and three to twelve months is most desirable. Exemplary synthetic polymers which may be used to form the biodegradable delivery system include: polymers of lactic acid and glycolic acid, polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyanhydrides, polyurethanes and co-polymers thereof, poly(butic acid), poly(valeric acid), alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone. Exemplary natural polymers include alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The polymer optionally is in the form of a hydrogel (see, for example, WO 04/009664, WO 05/087201, Sawhney, et al., Macromolecules, 1993, 26, 581-587) that can absorb up to about 90% of its weight in water and further, optionally is cross-linked with multi-valent ions or other polymers.

Delivery systems also include non-polymer systems that are lipids including sterols such as cholesterol, cholesterol esters and fatty acids or neutral fats such as mono-di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which the product is contained in a form within a matrix such as those described in U.S. Pat. Nos. 4,452,775, 4,675,189 and 5,736,152 and (b) diffusional systems in which a product permeates at a controlled rate from a polymer such as described in U.S. Pat. Nos. 3,854,480, 5,133,974 and 5,407,686. Liposomes containing the product may be prepared by methods known methods, such as for example (DE 3,218,121; Epstein et al., Proc. Natl. Acad. Sci. USA, 82: 3688-3692 (1985); Hwang et al., Proc. Natl. Acad. Sci. USA, 77: 4030-4034 (1980); EP 52,322; EP 36,676; EP 88,046; EP 143,949; EP 142,641; JP 83-118008; U.S. Pat. Nos. 4,485,045 and 4,544,545; and EP 102,324).

Alternatively or additionally, the compositions may be administered locally via implantation into the affected area of a membrane, sponge, or other appropriate material on to which an antibody comprising an ultralong CDR3, antibody fragment, nucleic acid, or vector disclosed herein has been absorbed or encapsulated. Where an implantation device is used, the device may be implanted into any suitable tissue or organ, and delivery of an antibody comprising an ultralong CDR3 antibody fragment, nucleic acid, or vector disclosed herein can be directly through the device via bolus, or via continuous administration, or via catheter using continuous infusion.

A pharmaceutical composition comprising an antibody comprising an ultralong CDR3, antibody fragment, nucleic acid, or vector disclosed herein may be formulated for inhalation, such as for example, as a dry powder. Inhalation solutions also may be formulated in a liquefied propellant for aerosol delivery. In yet another formulation, solutions may be nebulized. Additional pharmaceutical composition for pulmonary administration include, those described, for example, in WO 94/20069, which discloses pulmonary delivery of chemically modified proteins. For pulmonary delivery, the particle size should be suitable for delivery to the distal lung. For example, the particle size may be from 1 μm to 5 μm; however, larger particles may be used, for example, if each particle is fairly porous.

Certain formulations containing antibodies comprising an ultralong CDR3, antibody fragments, nucleic acids, or vectors disclosed herein may be administered orally. Formulations administered in this fashion may be formulated with or without those carriers customarily used in the compounding of solid dosage forms such as tablets and capsules. For example, a capsule can be designed to release the active portion of the formulation at the point in the gastrointestinal tract when bioavailability is maximized and pre-systemic degradation is minimized. Additional agents may be included to facilitate absorption of a selective binding agent. Diluents, flavorings, low melting point waxes, vegetable oils, lubricants, suspending agents, tablet disintegrating agents, and binders also can be employed.

Another preparation may involve an effective quantity of an antibody comprising an ultralong CDR3, antibody fragment, nucleic acid, or vector disclosed herein in a mixture with nontoxic excipients which are suitable for the manufacture of tablets. By dissolving the tablets in sterile water, or another appropriate vehicle, solutions may be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Suitable and/or preferred pharmaceutical formulations may be determined in view of the present disclosure and general knowledge of formulation technology, depending upon the intended route of administration, delivery format, and desired dosage. Regardless of the manner of administration, an effective dose may be calculated according to patient body weight, body surface area, or organ size. Further refinement of the calculations for determining the appropriate dosage for treatment involving each of the formulations described herein are routinely made in the art and is within the ambit of tasks routinely performed in the art. Appropriate dosages may be ascertained through use of appropriate dose-response data.

In some embodiments, antibodies comprising an ultralong CDR3 or fragments thereof are provided with a modified Fc region where a naturally-occurring Fc region is modified to increase the half-life of the antibody or fragment in a biological environment, for example, the serum half-life or a half-life measured by an in vitro assay. Methods for altering the original form of a Fc region of an IgG also are described in U.S. Pat. No. 6,998,253.

In certain embodiments, it may be desirable to modify the antibody or fragment in order to increase its serum half-life, for example, adding molecules such as PEG or other water soluble polymers, including polysaccharide polymers, to antibody fragments to increase the half-life. This may also be achieved, for example, by incorporation of a salvage receptor binding epitope into the antibody fragment (e.g., by mutation of the appropriate region in the antibody fragment or by incorporating the epitope into a peptide tag that is then fused to the antibody fragment at either end or in the middle, e.g., by DNA or peptide synthesis) (see, International Publication No. WO96/32478). Salvage receptor binding epitope refers to an epitope of the Fc region of an IgG molecule (e.g., IgG1, IgG2, IgG3, or IgG4) that is responsible for increasing the in vivo serum half-life of the IgG molecule.

A salvage receptor binding epitope may include a region wherein any one or more amino acid residues from one or two loops of an Fc domain are transferred to an analogous position of the antibody fragment. Even more preferably, three or more residues from one or two loops of the Fc domain are transferred. Still more preferred, the epitope is taken from the CH2 domain of the Fc region (e.g., of an IgG) and transferred to the CH1, CH3, or VH region, or more than one such region, of the antibody. Alternatively, the epitope is taken from the CH2 domain of the Fc region and transferred to the CL region or VL region, or both, of the antibody fragment. See also WO 97/34631 and WO 96/32478 which describe Fc variants and their interaction with the salvage receptor.

### IV. Methods of Treatment and Uses

Provided herein are methods for using and uses of the compositions containing a chimeric cytokine modified antibody or antigen binding fragment for treating a disease or condition. In particular embodiments, the disease or condition is one that is treatable with the cytokine present in the chimeric molecule. For example, the disease or condition is treatable with IL-2 or IL-15. In some embodiments, the provided chimeric cytokine modified antibodies or antigen binding fragments are particularly suitable for use as an immunotherapy. In particular aspects, the provided chimeric cytokine modified antibodies or antigen-binding fragments, or compositions thereof, have use in a number of oncology applications, such as cancer, by promoting T cell activation and/or proliferation. In some embodiments, the provided chimeric cytokine modified antibody or antigen binding fragment are use for treating cancer in a subject in need thereof.

Such methods and uses include therapeutic methods and uses, for example, involving administration of the molecules to a subject having a disease, condition or disorder, such as a cancer, to effect treatment of the disease or disorder. Uses include uses of the compositions in such methods and treatments, and uses of such compositions in the preparation of a medicament in order to carry out such therapeutic methods. In some embodiments, the methods and uses thereby treat the disease or condition or disorder, such as a tumor or cancer, in the subject.

In some embodiments, the cancer is a cancer of the head and neck, breast, liver, colon, ovary, prostate, pancreas, brain, cervix, bone, skin, lung, or blood. In some embodiments, cancer may include a malignant tumor characterized by abnormal or uncontrolled cell growth. Other features that may be associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. Metastatic disease may refer to cancer cells that have left the original tumor site and migrated to other parts of the body, for example via the bloodstream or lymph system.

In some embodiments, the provided methods result in an amelioration of and or treat the disease or condition, such as cancer. In some aspects, the provided methods result in one or more improvements in the disease, such as a reduction in the number of neoplastic cells, an increase in neoplastic cell death, inhibiting of neoplastic cell survival, inhibition (i.e. slowing to some extent or halting) of tumor growth, an increase in patient survival rate, and/or some relief from one or more symptoms associated with the disease or condition.

In aspects of the provided methods, response can be assessed or determined using criteria specific to the disease or condition. In some embodiments, tumor response can be assessed for changes in tumor morphology (i.e. overall tumor burden, tumor size) using screening techniques such as magnetic resonance imaging (MRI) scan, x-radiographic imaging, computed tomographic (CT) scan, bone scan imaging, endoscopy, and tumor biopsy sampling including bone marrow aspiration (BMA) and counting of tumor cells in the circulation.

The provided methods involve administering a therapeutically effection amount of the compositions provided herein to a subject in need thereof, such as a cancer subject. A therapeutically effective amount may vary according to factors such as the disease state age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects. In some cases, a therapeutically effective amount for tumor or cancer therapy may also be measured by its ability to stabilize the progression of disease. The ability of the provided antibody or antigen binding fragments to inhibit cancer may be evaluated in an animal model system predictive of efficacy in human tumors.

Alternatively, this property of a composition may be evaluated by examining the ability of the antibody or antigen binding fragment to inhibit cell growth or to induce apoptosis by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected.

In some embodiments, the provided antibodies or antigen binding fragments can be administered in a single dose, or in several doses, as needed to obtain the desired response. In some embodiments, the effective amount is dependent on the source applied, the subject being treated, the severity and type of the condition being treated, and the manner of administration.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

In some embodiments, the therapeutically effective amount is between at or about 0.1 to 100 mg/kg, or any value between any of the foregoing.

### V. Exemplary Embodiments

Among the provided embodiments are:

1. A chimeric cytokine modified antibody or antigen binding fragment, comprising a modified ultralong CDR3 comprising an interleukin-15 (IL-15) cytokine sequence or a biologically active portion thereof that replaces at least a portion of an ultralong CDR3 region of a heavy chain of a bovine antibody or antigen-binding fragment or a humanized sequence thereof.

2. The chimeric cytokine modified antibody or antigen binding fragment of embodiment 1, wherein the IL-15 cytokine sequence is human IL-15.

3. The chimeric cytokine modified antibody or antigen binding fragment of embodiment 1 or embodiment 2, wherein the IL-15 cytokine sequence comprises a sequence of amino acids that exhibits at least at or about 85%, at least at or about 90%, at least at or about 92%, at least at or about 95%, at least at or about 96%, at least at or about 97%, at least at or about 98%, or at least at or about 99% sequence identity to SEQ ID NO: 1.

4. The chimeric cytokine modified antibody or antigen binding fragment of any of embodiments 1-3 wherein the IL-15 cytokine sequence comprises the sequence of amino acids set forth in SEQ ID NO:1.

5. A chimeric cytokine modified antibody or antigen binding fragment, comprising a modified ultralong CDR3 comprising an interleukin-2 (IL-2) cytokine sequence or a biologically active portion thereof that replaces at least a portion of an ultralong CDR3 region of a heavy chain of a bovine antibody or antigen-binding fragment or a humanized sequence thereof.

6. The chimeric cytokine modified antibody or antigen binding fragment of embodiment 5, wherein the IL-2 cytokine sequence is human IL-2.

7. The chimeric cytokine modified antibody or antigen binding fragment of embodiment 5 or embodiment 6, wherein the IL-2 cytokine sequence comprises a sequence of amino acids that exhibits at least at or about 85%, at least at or about 90%, at least at or about 92%, at least at or about 95%, at least at or about 96%, at least at or about 97%, at least at or about 98%, or at least at or about 99% sequence identity to SEQ ID NO: 165.

8. The chimeric cytokine modified antibody or antigen binding fragment of any of embodiments 5-7 wherein the IL-2 cytokine sequence comprises the sequence of amino acids set forth in SEQ ID NO:165.

9. The chimeric cytokine modified antibody or antigen binding fragment of any of embodiments 1-8, wherein the cytokine sequence replaces at least a portion of an ultralong CDR3 region of a heavy chain of a bovine antibody or antigen-binding fragment.

10. The chimeric cytokine modified antibody or antigen binding fragment of embodiment 9, wherein the bovine antibody or antigen-binding fragment is the bovine antibody BLV1H12 or an antigen-binding fragment thereof.

11. The chimeric cytokine modified antibody or antigen binding fragment of embodiment 9 or embodiment 10, wherein the bovine antibody or antigen-binding fragment comprises a variable heavy chain amino acid sequence encoded by the sequence set forth in SEQ ID NO:5 and a variable light chain amino acid sequence encoded by the sequence set forth in SEQ ID NO:8.

12. The chimeric cytokine modified antibody or antigen binding fragment of embodiment 9 or embodiment 10, wherein the bovine antibody or antigen-binding fragment comprises a variable heavy chain amino acid sequence encoded by the sequence set forth in SEQ ID NO:167 and a variable light chain amino acid sequence encoded by the sequence set forth in SEQ ID NO: 168.

13. The chimeric cytokine modified antibody or antigen binding fragment of embodiment 9 or embodiment 10, wherein the bovine antibody or antigen-binding fragment comprises a variable heavy chain set forth in SEQ ID NO: 26 and a variable light chain set forth in SEQ ID NO: 27.

14. The chimeric cytokine modified antibody or antigen binding fragment of any of embodiments 1-8, wherein the cytokine sequence replaces at least a portion of an ultralong CDR3 region of a heavy chain of a humanized bovine antibody or antigen-binding fragment thereof.

15. The chimeric cytokine modified antibody or antigen binding fragment of embodiment 14, wherein the humanized bovine antibody or antigen-binding fragment thereof comprises a heavy chain or portion thereof that is a human heavy chain germline sequence or is derived from a human heavy chain germline sequence and a light chain or a portion thereof that is a human light chain germline sequence or is derived from a human light chain germline sequence.

16. The chimeric cytokine modified antibody or antigen binding fragment of embodiment 15, wherein the human heavy chain germline sequence is a VH4-39, VH4-59*03, VH4-34*02 or VH4-34*09 germline sequence or is a sequence set forth in any one of SEQ ID NOS: 68-71.

17. The chimeric cytokine modified antibody or antigen binding fragment of embodiment 15 or embodiment 16, wherein the human light chain germline sequence is a VL1-51 germline sequence or is a sequence based on the VL1-51 germline sequence comprising one or more mutations, optionally wherein the VL1-51 germline sequence is set forth in SEQ ID NO: 156.

18. The chimeric cytokine modified antibody or antigen binding fragment of embodiment 17, wherein the one or more mutations are selected from among:

one or more of amino acid replacements S2A, T5N, P8S, A12G, A13S, and P14L based on Kabat numbering;

amino acid replacements S2A, T5N, P8S, A12G, A13S, and P14L based on Kabat numbering;

mutations in CDR1 comprising amino acid replacements 129V and N32G;

mutations in CDR2 comprising a substitution of DNN to GDT;

mutations in CDR2 comprising a substitution DNNKRP to GDTSRA;

or a combination of any of the forgoing.

19. The chimeric cytokine modified antibody or antigen binding fragment of any of embodiments 1-18, wherein the antibody is an antigen-binding fragment comprising a variable heavy chain and a variable light chain.

20. The chimeric cytokine modified antibody or antigen binding fragment of any of embodiments 1-19, wherein the antibody comprises a variable heavy chain joined to a heavy chain constant domain (CH1-CH2-CH3) and a variable light chain joined to a light chain constant domain (CL1).

21. The chimeric cytokine modified antibody or antigen binding fragment of embodiment 20, wherein the heavy chain constant domain is from a human IgG1.

22. The chimeric cytokine modified antibody or antigen binding fragment of embodiment 20 or embodiment 21, wherein the light chain constant domain is a lambda light chain region.

23. The chimeric cytokine modified antibody or antigen binding fragment of any of embodiments 1-22, wherein the at least a portion of an ultralong CDR3 region comprises the knob region and the cytokine sequence is present between the ascending stalk domain and the descending stalk domain of the modified ultralong CDR3.

24. The chimeric cytokine modified antibody or antigen binding fragment of embodiment 23, wherein the cytokine sequence is linked to the ascending stalk domain and/or the descending stalk domain via a flexible linker, optionally a GGS or GSG linker.

25. The chimeric cytokine modified antibody or antigen binding fragment of embodiment 23 or embodiment 24, wherein the ascending stalk domain comprises the sequence set forth in SEQ ID NO:158 or SEQ ID NO:159.

26. The chimeric cytokine modified antibody or antigen binding fragment of any of embodiments 23-25, wherein the descending stalk domain comprises the sequence set forth in SEQ ID NO:161.

27. The chimeric cytokine modified antibody or antigen binding fragment of any of embodiments 1-4 and 9-26, wherein the antibody or antigen binding fragment comprises a variable heavy chain sequence encoded by the sequence of nucleotides set forth in SEQ ID NO:7 or a sequence of nucleotides that exhibits at least at or about 85%, at least at or about 90%, at least at or about 92%, at least at or about 95%, at least at or about 96%, at least at or about 97%, at least at or about 98%, at least at or about 99% sequence identity to the nucleotide sequence set forth in SEQ ID NO:7, in which is contained a modified ultralong CDR3 containing an IL-15 sequence.

28. The chimeric cytokine modified antibody or antigen binding fragment of any of embodiments 1-4 and 9-27, wherein the antibody or antigen binding fragment is complexed with an extracellular domain of the IL15Rα comprising the IL15Rα sushi domain.

29. The chimeric cytokine modified antibody or antigen binding fragment of embodiment 28, wherein the extracellular domain of the IL15Rα comprising the IL15Rα sushi domain is non-covalently associated with the IL-15 sequence.

30. The chimeric cytokine modified antibody or antigen binding fragment of embodiment 28, wherein the extracellular domain of the IL15Rα comprising the IL15Rα sushi domain is linked to the variable light chain.

31. The chimeric cytokine modified antibody or antigen binding fragment of embodiment 30 that is linked via a peptide linker.

32. The chimeric cytokine modified antibody of embodiment 31, wherein the peptide linker is a glycine linker or a glycine-serine linker, optionally wherein the linker is GS.

33. The chimeric cytokine modified antibody of any of embodiments 28-32, wherein the extracellular domain of the IL15Rα comprising the IL15Rα sushi domain comprises the sequence set forth in SEQ ID NO:2.

34. The chimeric cytokine modified antibody or antigen binding fragment of any of embodiments 30-33, wherein the variable light chain comprises the sequence of amino acids encoded by SEQ ID NO:3.

35. A polynucleotide(s) encoding a chimeric cytokine modified antibody or antigen binding fragment of any of embodiments 1-34.

36. A polynucleotide encoding a heavy chain or a variable region thereof of a chimeric cytokine modified antibody or antigen binding fragment of any of embodiments 1-34.

37. A polynucleotide encoding a light chain or a variable region thereof of a chimeric cytokine modified antibody or antigen binding fragment of any of embodiments 1-34.

38. An expression vector comprising the polynucleotide of any of embodiments 35-37.

39. A host cell comprising the polynucleotide of any of embodiments 35-37 or the expression vector of embodiment 38.

40. The host cell of embodiment 39, further comprising a polynucleotide or vector expressing an extracellular domain of the IL15Rα comprising the IL15Rα sushi domain.

41. The host cell of embodiment 40, wherein the extracellular domain of the IL15Rα comprising the IL15Rα sushi domain comprises the sequence set forth in SEQ ID NO:2.

42. A method of producing a chimeric cytokine modified antibody or antigen binding fragment comprising culturing the host cell of any of embodiments 39-41 under conditions for expression of the antibody or antigen binding fragment by the cell, optionally further comprising recovering of purifying the antibody or antigen binding fragment.

43. A chimeric cytokine modified antibody or antigen binding fragment produced by the method of embodiment 42.

44. A pharmaceutical composition comprising the chimeric cytokine modified antibody or antigen binding fragment of any of embodiments 1-34 or 43.

45. A method of treating a cancer in a subject, comprising administering a therapeutically effective amount of a chimeric cytokine modified antibody or antigen binding fragment of any of embodiments 1-34 or 43.

46. A method of treating a cancer in a subject, comprising administering a therapeutically effective amount of a pharmaceutical composition of embodiment 44.

### VI. Examples

The following examples are included for illustrative purposes only and are not intended to limit the scope of the invention.

### Example 1 Generation of Chimeric Interleukin 15 Fusion Antibodies

Chimeric BLV1H12-IL-15 (B15) fusion antibodies were generated in which the ultralong CDR3 region of BLV1H12 was engineered by replacing the knob region of the bovine BLV1H12 antibody with interleukin (IL)-15.

The variable heavy (VH) region from a chimeric BLV1H12 bovine heavy sequence (SEQ ID NO:167) was amplified by PCR and subcloned in-frame between the signal sequence and nucleotide sequence encoding CH1-CH2-CH3 of human lgG1 to produce a sequence set forth in SEQ ID NO: 6. The chimeric ultralong bovine heavy sequence (SEQ ID NO: 167) contains the stalk sequences from the heavy chain of BLV1H12 where the last serine in the ascending stalk strand was changed to threonine for cloning purposes, and contains a knob sequence from a bovine anti-HIV antibody. To insert an IL-15 cytokine sequence (set forth in SEQ ID NO: 1) into the CDR3 of the chimeric BLV1H12 heavy chain, a sequence (SEQ ID NO: 7) encoding the entire B15 variable region and its signal peptide was designed by replacing the knob sequence (SEQ ID NO: 162) with the IL-15 sequence together with sequences encoding for a N-terminal GGS linker (SEQ ID NO: 163) and a C-terminal GSG linker (SEQ ID NO: 164) where IL-15 connects with the ascending (SEQ ID NO: 157 encoding the sequence set forth in SEQ ID NO:159) and descending stalks (SEQ ID NO: 160 encoding the sequence set forth in SEQ ID NO: 161). This sequence was chemically synthesized with a 5′ EcoRI site and cloned into pUC57 vector by GenScript, Inc. A 3′ end NheI site already existed in the synthesized sequence. The synthesized sequence was subcloned into BLV1H12 expression vector (SEQ ID NO: 6) using EcoRI and NheI restriction enzymes.

The expression vector encoding each heavy chain was then co-transfected in parallel with pFUSE expression vector encoding the a bovine light chain BLV1H12 (SEQ ID NO: 168) into freestyle HEK 293 cells (ThermoScientific). The cells were allowed to grow at 37° C., 8% CO₂ and expressed chimeric BLV1H12-IL-15 (B15) fusion antibodies were secreted into the culture medium and harvested at 96 hours after transfection. Chimeric fusion antibodies were purified by CaptureSelect CH1-XL affinity matrix (ThermoScientific), then concentrated and buffer exchanged into phosphate buffered saline (PBS) using Amicon Ultra-4 centrifugal filters (MW cutoff=10,000 kDa, Millipore Sigma). They were quantified using Nanodrop based on the molecular weight and extinction coefficient.

To assess if IL-15 may need its high affinity receptor α (IL15Rα) for increased trans signaling to the receptor β and γ subunits (IL2/15Rβ and γc), two additional molecules were produced by co-expression of the IL-15 chimeric fusion antibodies with the sushi domain of IL15Rα. The two additional variant molecules were produced by either co-expressing IL15Rα sushi domain (SEQ ID NO: 2) with the chimeric IgG in freestyle HEK 293 cells (B15_Rαsushi) or fusing the IL15Rα sushi domain to the light chain through a GS linker (SEQ ID NO: 3) (B15_GS_Rαsushi).

FIG. 1A and FIG. 1B set forth schematic depictions of the generated constructs.

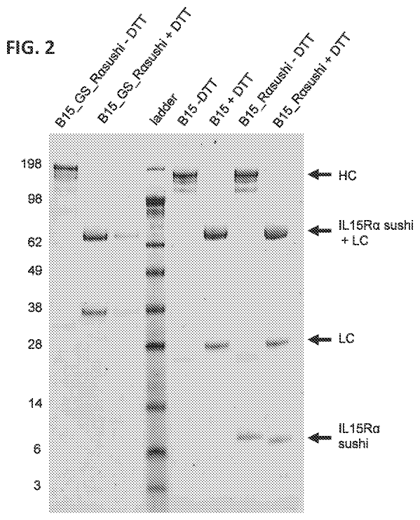

B15 fusion antibodies were analyzed by the SDS-PAGE gel. FIG. 2 shows an SDS-PAGE gel of purified B15 fusion antibody constructs BLV1H12-IL-15 (B15), BLV1H12-IL-15-Rαsushi (B15_Rαsushi) and BLV1H12-IL-15-GS-Rαsushi (B15_GS_Rαsushi) expressed from HEK 293 cells. These results demonstrate that chimeric B15 antibodies or the variants containing the IL15-Rαsushi domain could be expressed and purified similarly to typical human antibodies.

### Example 2 Chimeric B15 Fusion Antibody-Receptor Binding Assays

Binding of chimeric BLV1H12-IL-15 (B15) fusion antibodies to the IL2 receptor α (IL2Rα) and IL15Rα was evaluated in an enzyme-linked immunosorbent assay (ELISA). 50 ng IL2Rα or 100 ng IL15Rα proteins (R&D systems) were coated per well in a 96-well high binding plate at 4° C. overnight. The plate was washed three times with tris buffered saline (TBS) containing 0.1% Tween 20 (TBST). Unbound sites on the plate were blocked with 1% bovine serum albumin (BSA) prepared in TBST at room temperature for 1 hour. 10 picomole B15 (diluted in 1% BSA in TBST) was added per well, and negative control wells were also set up with only BSA added. The plate was incubated at room temperature for 1 hour, then it was washed four times with TBST to remove unbound B15. Detection antibody used was horseradish peroxidase conjugated goat anti-human lambda (Southern Biotech), which was diluted 1 to 5000 in 1% BSA in TBST, and 50 ul dilution was added per well. After 30 minutes incubation with the secondary antibody, the plate was washed five times with TBST to remove unbound secondary antibodies. 50 ul TMB substrate (TheromoScientific) was added per well and the horseradish peroxidase—TMB reaction were ran for 1 minute and 30 seconds and then stopped by adding 50 ul per well 1.0 Normality sulfuric acid. Plates were read at 450 nm in a Tecan plate reader and values plotted were averages of three duplicate wells with background readings deducted.

Binding of chimeric BLV1H12-IL-15 (B15) fusion antibody to the IL2/15Rβ receptor was evaluated in an ELISA assay. The plate was coated with 50 ng per well IL2/15Rβ proteins (R&D systems) at 4° C. overnight. The plate was washed three times with tris buffered saline (TBS) containing 0.1% Tween 20 (TBST). Unbound sites on the plate were blocked with 1% bovine serum albumin (BSA) prepared in TBST at room temperature for 1 hour. 10 picomole B15 or premixed equal molar B15 and IL15Rα-Fc (R&D systems) was added per well, and negative control wells were also set up with only BSA added. The plate was incubated at room temperature for 1 hour, and it was then washed four times with TBST to remove unbound B15 or premixed B15 and IL15Rα-Fc. Detection antibody used was horseradish peroxidase conjugated goat anti-human lambda (Southern Biotech), which was diluted 1 to 5000 in 1% BSA in TBST, and 50 ul dilution was added per well. After 30 minutes incubation with the secondary antibody, the plate was washed five times with TBST to remove unbound secondary antibodies. 50 ul TMB substrate (TheromoScientific) was added per well and the peroxidase—TMB reaction were ran for 3 minutes and then stopped by adding 50 ul per well 1.0 Normality sulfuric acid. Plates were read at 450 nm in a Tecan plate reader and values plotted were averages of three duplicate wells with background readings deducted.

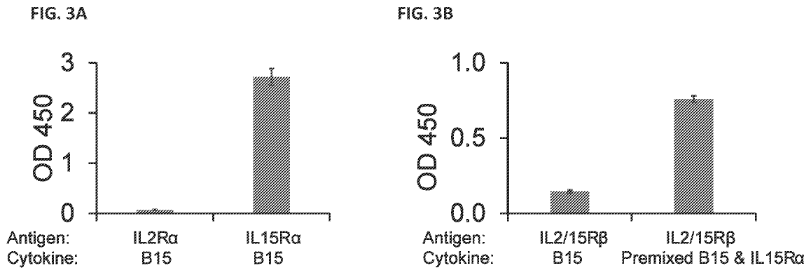

As shown in FIGS. 3A and 3B, chimeric B15 could bind to both IL15Rα and IL2/15Rβ subunits, and the IL15Rα sushi domain subunit could improve B15 binding to the IL2/15Rβ subunit. No binding between B15 and IL2Rα was detected. These results demonstrated that the IL15Rα or its sushi domain is involved in efficient binding to IL2/15Rβ and γc subunits.

### Example 3 Chimeric B15 Fusion Antibody Induced Receptor Activation and Signaling

Activation of the IL2/15Rβ and γc receptor and STAT5 signaling by chimeric B15 molecules, generated as described in Example 1, was tested using HEK-Blue IL2 reporter cells (InvivoGen), and analyzed through induction and secretion of the STAT5 inducible alkaline phosphatase (SEAP) reporter gene.

As there is no IL15Rα subunit expressed in the HEK-Blue IL2 reporter cells, IL15Rα-Fc (R&D Systems) was mixed with IL15 (or B15) to increase its binding to the IL2/15Rβ and γc subunits. First, HEK-Blue IL2 reporter cells were prepared into suspension by gently rinsing cells twice with pre-warmed phosphate buffered saline (PBS), detaching the cells in presence of PBS by using a cell scraper, and resuspending cells in fresh, pre-warmed test medium (DMEM with high glucose and 10% heat-inactivated FBS) to ˜280,000 cells per ml. IL15 monomer incubated with half molar of IL15Rα-Fc either at 4° C. overnight (Premixed IL15 & IL15Rα) or just prior to the initiation of the assay (Freshly mixed IL15 & IL15Rα), or chimeric B15 mixed with equal molar of IL15Rα-Fc just prior to initiation of the assay (Freshly mixed B15 & IL15Rα) were 4-fold serially diluted in PBS from 64 nM to 0.25 nM, and 20 ul of each cytokine dilution was added per well to a 96-well tissue culture treated plate with three replicates per dilution. 50,000 cells were then added to each well and cultured at 37° C., 5% CO₂ for 20 hours. Because chimeric B15 antibodies are bivalent, only half-molar concentrations were used compared to IL15 monomers. 20 ul cell culture supernatants from each well containing secreted SEAP were mixed with 180 ul Quanti-Blue substrate solution at 37° C. for 30 minutes, the color changes (corresponding to amount of SEAP secreted) were measured using Tecan plate reader at 590 nm.

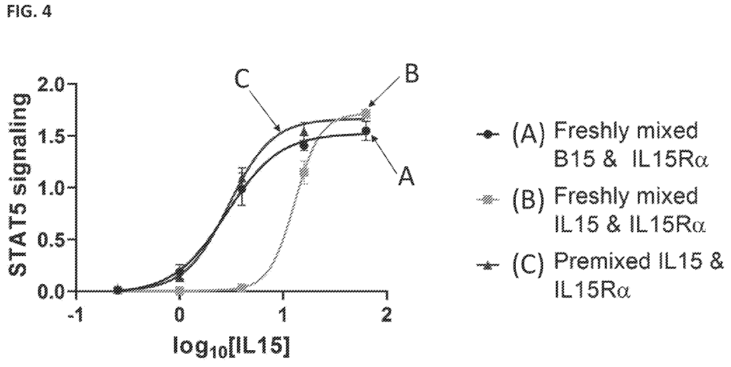

As shown in FIG. 4, the in vitro STAT5 signaling assay indicated that chimeric B15 antibodies could associate with the IL2/15Rβ receptor much faster than IL15 monomers.

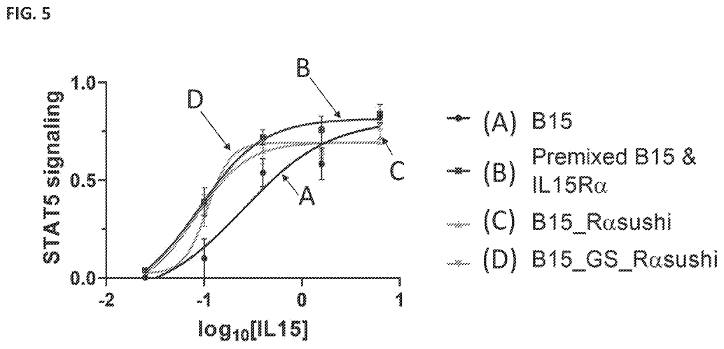

HEK-Blue IL2 reporter cells were then used to assess receptor activation and STAT5 signaling in the presence of the alternative chimeric B15 molecules that were associated with the IL15Rαsushi domain. HEK-Blue IL2 reporter cells were prepared the same as above and were co-cultured with 4-fold serially diluted (from 64 nM to 0.25 nM) chimeric B15 antibodies alone, chimeric B15 antibodies mixed with an IL15Rα-Fc just prior to initiation of the assay (Freshly mixed B15 & IL15Rα), chimeric B15 variant B15_Rαsushi, or chimeric B15 variant B15_GS_Rαsushi antibodies. As shown in FIG. 5, chimeric B15 variants expressed with IL15Rα sushi domain achieved the same signaling potency as premixed B15 and IL15Rα-Fc, which were all better than chimeric B15 antibodies in the absence of the IL15Rα subunit.

### Example 4 Assessment of the Activity of Chimeric B15 Fusion Antibodies by Expansion of NK-92 Cells

The activity of chimeric B15 molecules, generated as described in Example 1, was assessed by their ability to expand NK-92 natural killer cells. NK-92 cells express IL2Rα, IL15Rα, IL2/15Rβ and γc subunits, and their growth and proliferation are dependent on the exogenous addition of IL2 or IL15 to bind and activate the receptors.

NK-92 cells were maintained in growth medium supplied with 200 U/ml of IL2. Prior to the expansion assays, NK-92 cells were washed twice with the growth medium without IL2 to get rid of any residual cell bound IL2, and 10,000 cells were seeded per well in a tissue culture treated 96-well plate. These cells were incubated with 2-fold serially diluted (from 1.33 nM to 0.005 nM) of IL2 or IL15 monomers (R&D Systems), or chimeric B15, chimeric variant B15_Rαsushi, or chimeric B15 variant B15_GS_Rαsushi antibodies at 37° C., 5% CO₂ for 48 hours. For chimeric B15 antibodies and its variants, only half-molar concentrations were used compared to IL2 and IL15 monomers. Final NK92 cell number per well was assessed by the reduction of the tetrazolium dye MTT to its insoluble formazan by the presence of metabolically active oxidoreductase enzymes (MTT assay kit, Promega).

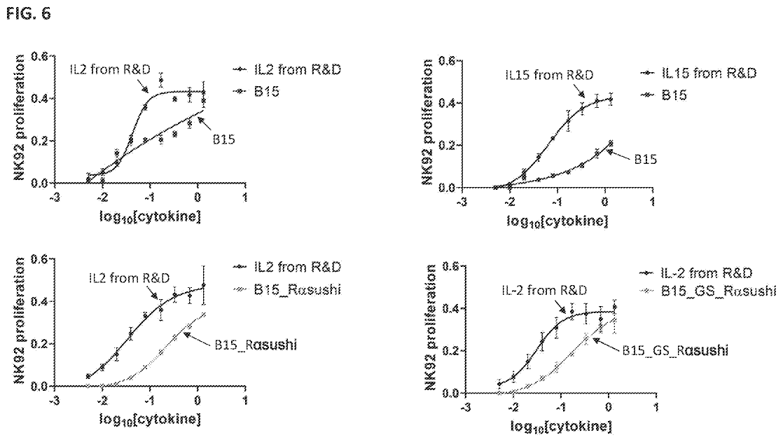

As shown in FIG. 6, all B15 constructs were capable of expanding NK-92 cells, although to a lesser extent than either IL2 or IL15 monomers. It was unknown why chimeric B15 or its variants with IL15Rαsushi was less potent in expanding NK-92 cells. Without wishing to be bound by theory, one hypothesis is that although chimeric B15 or its variants are bivalent, they could only bind monovalently on the NK-92 cells, while only half molar concentrations of the chimeric B15 or its variants were used these assays. A second hypothesis is that chimeric B15 and its variants were produced in HEK cells and were naturally glycosylated compared to the E. coli produced IL2 and IL15 monomers (R&D systems), and glycosylation of IL15 may have a negative effect on its binding to the IL15 receptors on NK-92 cells. A third hypothesis is that the size of chimeric B15 or its variants is larger than IL2 or IL15 monomers due to its fusion to an antibody structure, which stabilizes the IL15 but decreases its accessibility to the IL15 receptors on NK-92 cells.

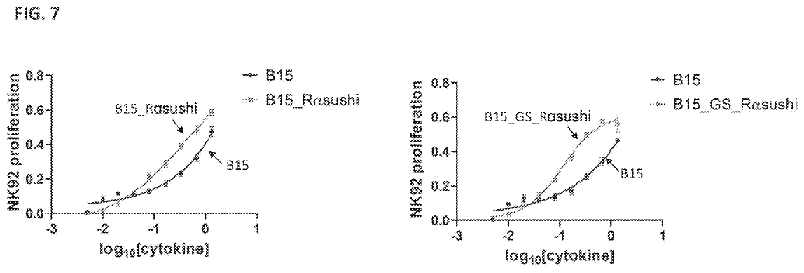

Expansion of NK-92 cells was then used to assess the difference in activity of chimeric B15 antibodies compared to chimeric B15 variants B15-Rαsushi or B15-GS-Rαsushi antibodies. Experiments were set up the same way as in FIG. 6. As shown in FIG. 7, the presence of the IL15Rα sushi domain improved the ability of the chimeric B15 antibodies to expand NK-92 cells.

### Example 5 Generation of Chimeric Interleukin 2 Fusion Antibody

Chimeric BLV1H12-IL-2 (B2) fusion antibody was generated by replacing the IL15 region of the chimeric B15 antibody described above with IL-2 (SEQ ID NO: 165).

IL2 coding sequence (SEQ ID NO: 166) with a 5′ end GGS linker coding sequence (SEQ ID NO: 163) and a 3′ end GSG linker coding sequence (SEQ ID NO: 164) was chemically synthesized by GenScript Inc. A 5′ end AgeI site and a 3′ end BamH site were also added. The synthesized sequence was cloned into pUC57 vector by GenScript, Inc., and was subcloned into chimeric B15 heavy chain variable region (SEQ ID NO: 7) using AgeI and BamHI restriction enzymes.

The expression vector encoding the heavy chain was then co-transfected in parallel with pFUSE expression vector encoding the a bovine light chain BLV1H12 (SEQ ID NO: 168) into freestyle HEK 293 cells (ThermoScientific.). The cells were allowed to grow at 37° C., 8% CO₂ and expressed chimeric BLV1H12-IL-2 (B2) fusion antibodies were secreted into the culture medium and harvested at 96 hours after transfection. Chimeric B2 fusion antibodies were purified by CaptureSelect CH1-XL affinity matrix (ThermoScientific), then concentrated and buffer exchanged into phosphate buffered saline (PBS) using Amicon Ultra-4 centrifugal filters (MW cutoff=10,000 kDa, Millipore Sigma). They were quantified using Nanodrop based on the molecular weight and extinction coefficient.

FIG. 8A and FIG. 8B set forth schematic depictions of the generated constructs.

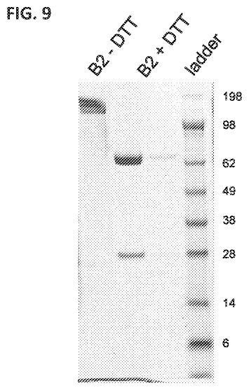

B2 fusion antibodies were analyzed by the SDS-PAGE gel. FIG. 9 shows an SDS-PAGE gel of purified fusion antibody constructs BLV1H12-IL-2 (B2), expressed from HEK 293 cells. The result demonstrates that the chimeric B2 antibody could be expressed and purified similarly to typical human antibodies.

### Example 6 Chimeric B2 Fusion Antibody-Receptor Binding Assays

Binding of chimeric BLV1H12-IL-2 (B2) fusion antibodies to the IL2Rα and IL15Rα was evaluated in an enzyme-linked immunosorbent assay (ELISA). 50 ng IL2Rα or 100 ng IL15Rα proteins (R&D systems) were coated per well in a 96-well high binding plate at 4° C. overnight. The next day, the plate was washed three times with tris buffered saline (TBS) containing 0.1% Tween 20 (TBST). Unbound sites on the plate were blocked with 1% bovine serum albumin (BSA) prepared in TBST at room temperature for 1 hour. 10 picomole B2 (diluted in 1% BSA in TBST) was added per well, and negative control wells were also set up with only BSA added. The plate was incubated at room temperature for 1 hour and was then washed four times with TBST to remove unbound B2 antibodies. Detection antibody used was horseradish peroxidase conjugated goat anti-human lambda (Southern Biotech), which was diluted 1 to 5000 in 1% BSA in TBST, and 50 ul dilution was added per well. After 30 minutes incubation with the secondary antibody, the plate was washed five times with TBST to remove unbound secondary antibodies. 50 ul TMB substrate (TheromoScientific) was added per well and the horseradish peroxidase—TMB reaction were ran for 1 minute and 30 seconds and then stopped by adding 50 ul per well 1.0 Normality sulfuric acid. Plates were read at 450 nm in a Tecan plate reader and values plotted were averages of three duplicate wells with background readings deducted.

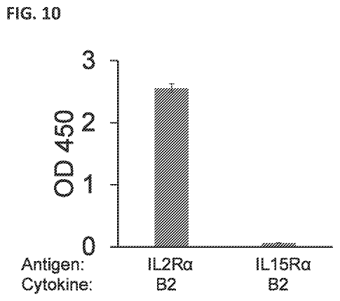

As shown in FIG. 10, chimeric B2 could bind to the IL2Rα but not the IL15Rα.

### Example 7 Chimeric B2 Fusion Antibody Induced Receptor Activation and Signaling

Activation of the IL2/15Rβ and γc receptor and STAT5 signaling by the chimeric B2 molecule, generated as described in Example 5, was tested using HEK-Blue IL2 reporter cells (InvivoGen) against IL2 monomers (R&D systems and Millipore Sigma), and analyzed through induction and secretion of the STAT5 inducible alkaline phosphatase (SEAP) reporter gene.

First, HEK-Blue IL2 reporter cells were prepared into suspension by gently rinsing cells twice with pre-warmed phosphate buffered saline (PBS), detaching the cells in presence of PBS by using a cell scraper, and resuspending cells in fresh, pre-warmed test medium (DMEM with high glucose and 10% heat-inactivated FBS) to ˜280,000 cells per ml. IL2 monomers or the chimeric B2 antibody were 4-fold serially diluted in PBS from 64 nM to 0.25 nM, and 20 ul of each cytokine dilution was added per well to a 96-well tissue culture treated plate with three replicates per dilution. 50,000 cells were then added to each well and cultured at 37° C., 5% CO₂ for 20 hours. Because the chimeric B2 antibody is bivalent, only half-molar concentrations were used compared to IL2 monomers. 20 ul cell culture supernatants from each well containing secreted SEAP were mixed with 180 ul Quanti-Blue substrate solution at 37° C. for 30 minutes, the color changes (corresponding to amount of SEAP secreted) were measured using Tecan plate reader at 590 nm.

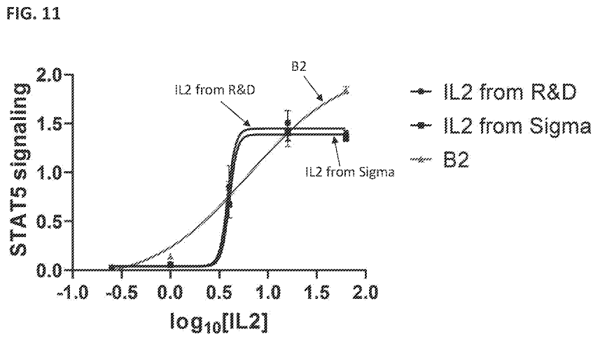

As shown in FIG. 11, the in vitro STAT5 signaling assay indicated that the chimeric B2 antibody performs similar to IL2 monomers derived from E. coli (R&D systems and Millipore Sigma).

### Example 8 Assessment of the Activity of Chimeric Fusion B2 Antibodies by Expansion of NK-92 Cells

The activity of chimeric B2 molecule, generated as described in Example 5, was assessed by its ability to expand NK-92 natural killer cells. NK-92 cells express IL2Rα, IL15Rα, IL2/15Rβ and ye subunits, and their growth and proliferation are dependent on the exogenous addition of IL2 or IL15 to bind and activate the receptors.

NK-92 cells were maintained in growth medium supplied with 200 U/ml of IL2. Prior to the expansion assays, NK-92 cells were washed twice with growth medium without IL2 to get rid of any residual cell bound IL2, and 10,000 cells were seeded per well in a tissue culture treated 96-well plate. These cells were incubated with 2-fold serially diluted (from 1.33 nM to 0.005 nM) of IL2 monomers (R&D Systems) or chimeric B2 antibodies at 37° C., 5% CO₂ for 48 hours. For the chimeric B2 antibody, only half-molar concentrations were used compared to IL2 monomers. Final NK92 cell number per well was assessed by the reduction of the tetrazolium dye MTT to its insoluble formazan by the presence of metabolically active oxidoreductase enzymes (MTT assay kit, Promega).

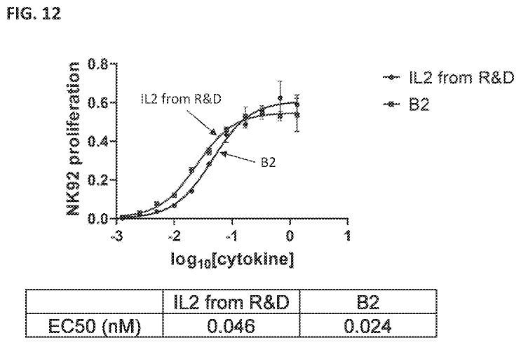

As shown in FIG. 12, chimeric B2 antibodies were almost two-fold better than IL2 monomers in NK-92 cell expansion.

### Example 9 Assessment of the In Vitro Activity of Chimeric B15 Fusion Antibodies in Human PBMCs

The activity of chimeric B15 molecules, generated as described in Example 1, was assessed by their ability to stimulate NK cells and T cells in human PBMCs in vitro. Both NK cells and T cells express IL15Rα, IL2/15Rβ and γc subunits, and their growth and proliferation are dependent on endogenous or exogenous IL15 to bind and activate the receptors.

Human PBMCs were washed in PBS twice, counted using hemocytometer and resuspended in RPMI1640 medium with 10% FBS. 100,000 cells in 100 μl were seeded per well in a tissue culture treated 96-well flat-bottom or U-bottom (facilitating cell contacts) plate. B15 and B15_Rαsushi were 5-fold serially diluted from 500 nM to 0.032 nM in the same medium and 100 μl of each dilution was added to the corresponding cells to achieve the final concentration from 250 nM to 0.016 nM. Controls were also set up without any B15 or B15_Rαsushi added. These cells were incubated at 37° C., 5% CO₂ for 96 hours. After treatment, PBMCs were stained with anti-CD3-FITC (SK7), anti-CD4-PE (OKT4), anti-CD8a-eFluor 450 (SKi) and anti-CD56-APC (AF12-7H3) to gate for the following cell types: CD3+CD4+ T cells, CD3+CD8+ T cells and NK cells (CD3-CD56+). Intracellular Ki67 as a cell proliferation marker was stained using anti-Ki67-PE-Cy7 (20Raj1) and Foxp3/Transcription Factor Staining Buffer Set (Thermo Fisher Scientific) following the manufacturer's protocol. Stained samples were subsequently analyzed using Novocyte Advanteon Flow Cytometer (Agilent, Santa Clara, Calif.).

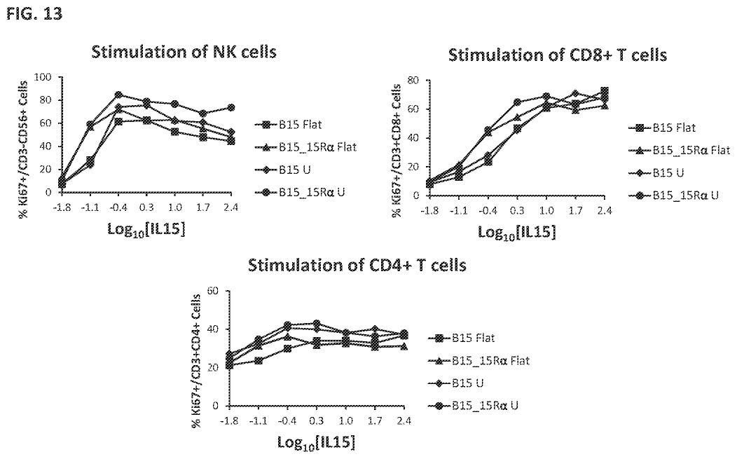

As shown in FIG. 13, both B15 and B15_15Rα induce potent proliferation of CD8+ T cells and NK cells in vitro, and to a much lesser extent CD4+ T cells. The proliferation is independent of different types of 96-well plates used (Flat vs. U-bottom), which suggests that the proliferation is solely induced by B15 or B15_15Rα with minimal effects from intercellular contacts. In these experiments, B15_15Rα performed slightly better than B15 at lower concentrations in inducing T cells and NK cells proliferation, suggesting that IL15Rα can boost IL15 functions at low concentrations. Proliferation of NK cells induced by B15 and B15_15Rα is plateaued at 0.4 nM, while proliferation of CD8+ T cells is plateaued at 10 nM, indicating that B15 and B15_15Rα have a higher affinity with NK cells than CD8+ T cells.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| SEQ | | |
|---|---|---|
| ID | | |
| NO | SEQUENCE | ANNOTATION |
| 1 | NWVNVISDLKKIEDLIQSMHIDATLYTESDVHPSCKVTAMKCFLLELQVIS | IL-15 |
| | L ESGDASIHDTVENLIILANNSLSSNGNVTESGCKECEELEEKNIKEFLQS | |
| | FVHIVQMFINTS | |
| 2 | ITCPPPMSVEHADIWVKSYSLYSRERYICNSGFKRKAGTSSLTECVLNKAT | IL15-Rαsushi |
| | NVAHWTTPSLKCIRDPALVHQRPAPP | |
| 3 | ATCACCTGCCCACCTCCAATGAGCGTGGAGCACGCAGACATCTGGGT | IL-15 Rα |
| | GAAGTCTTACAGCCTGTATTCCCGGGAGAGATACATCTGCAACTCTG | sushi_GSlinker_ |
| | GCTTCAAGCGGAAGGCCGGCACCAGCTCCCTGACAGAGTGCGTGCTG | BLV1H12 light |
| | AACAAGGCCACCAATGTGGCCCACTGGACAACTCCTTCCCTGAAATG | chain |
| | TATTAGAGACCCCGCCCTGGTGCATCAGAGACCTGCCCCCCCTGGTG | |
| | GAGGCGGTTCAGGCGGAGGTGGATCCCAGGCCGTCCTGAACCAGCCA | |
| | AGCAGCGTCTCCGGGTCTCTGGGGCAGCGGGTCTCAATCACCTGTAG | |
| | CGGGTCTTCCTCCAATGTCGGCAACGGCTACGTGTCTTGGTATCAGC | |
| | TGATCCCTGGCAGTGCCCCACGAACCCTGATCTACGGCGACACATCC | |
| | AGAGCTTCTGGGGTCCCCGATCGGTTCTCAGGGAGCAGATCCGGAAA | |
| | CACAGCTACTCTGACCATCAGCTCCCTGCAGGCTGAGGACGAAGCAG | |
| | ATTATTTCTGCGCATCTGCCGAGGACTCTAGTTCAAATGCCGTGTTT | |
| | GGAAGCGGCACCACACTGACAGTCCTAGGTCAGCCCAAGGCTGCCCC | |
| | CTCGGTCACTCTGTTCCCGCCCTCCTCTGAGGAGCTTCAAGCCAACA | |
| | AGGCCACACTGGTGTGTCTCATAAGTGACTTCTACCCGGGAGCCGTG | |
| | ACAGTGGCCTGGAAGGCAGATAGCAGCCCCGTCAAGGCGGGAGTGGA | |
| | GACCACCACACCCTCCAAACAAAGCAACAACAAGTACGCGGCCAGCA | |
| | GCTATCTGAGCCTGACGCCTGAGCAGTGGAAGTCCCACAGAAGCTAC | |
| | AGCTGCCAGGTCACGCATGAAGGGAGCACCGTGGAGAAGACAGTGGC | |
| | CCCTACAGAATGTTCATAA | |
| 4 | VNGTSQFTCFYNSRANISCVWSQDGALQDTSCQVHA | IL2 receptor |
| | WPDRRRWNQTCELLPVSQASWACNLILGAPDSQKL | subunit beta |
| | TTVDIVTLRVLCREGVRWRVMAIQDFKPFENLRLM | |
| | APISLQVVHVETHRCNISWEISQASHYFERHLEFE | |
| | ARTLSPGHTWEEAPLLTLKQKQEWICLETLTPDTQ | |
| | YEFQVRVKPLQGEFTTWSPWSQPLAFRTKPAALGK | |
| | DTIPWLGHLLVGLSGAFGFIILVYLLINCRNTGPW | |
| | LKKVLKCNTPDPSKFFSQLSSEHGGDVQKWLSSPF | |
| | PSSSFSPGGLAPEISPLEVLERDKVTQLLLQQDKV | |
| | PEPASLSSNHSLTSCFTNQGYFFFHLPDALEIEAC | |
| | QVYFTYDPYSEEDPDEGVAGAPTGSSPQPLQPLSG | |
| | EDDAYCTFPSRDDLLLFSPSLLGGPSPPSTAPGGS | |
| | GAGEERMPPSLQERVPRDWDPQPLGPPTPGVPDLV | |
| | DFQPPPELVLREAGEEVPDAGPREGVSFPWSRPPG | |
| | QGEFRALNARLPLNTDAYLSLQELQGQDPTHLV | |
| 5 | CAGGTCCAGC TGAGAGAGAG CGGCCCTTCA CTGGTCAAGC | BLV1H 12 |
| | CATCCCAGAC ACTGAGCCTGACATGCACAG CAAGCGGGTT | heavy chain |
| | TTCACTGAGC GACAAGGCAG TGGGATGGGT CCGACAGGCA | |
| | CCAGGAAAAG CCCTGGAATG GCTGGGCAGC ATCGATACCG | |
| | GCGGGAACAC AGGGTACAAT CCCGGACTGA AGAGCAGACT | |
| | GTCCATTACC AAGGACAACT CTAAAAGTCA GGTGTCACTG | |
| | AGCGTGAGCT CCGTCACCAC AGAGGATAGT GCAACTTACT | |
| | ATTGCACCTC TGTGCACCAG GAAACTAAGA AATACCAGAG | |
| | CTGTCCTGAC GGCTATCGGG AGAGATCTGA TTGCAGTAAT | |
| | AGGCCAGCTT GTGGCACATC CGACTGCTGT CGCGTGTCTG | |
| | TCTTCGGGAA CTGCCTGACT ACCCTGCCTG TGTCCTACTC | |
| | TTATACCTAC AATTATGAAT GGCATGTGGA TGTCTGGGGA | |
| | CAGGGCCTGC TGGTGACAGT CTCTAGTGCT AGC | |
| 6 | ATGGGATGGTCATGTATCATCCTTTTTCTAGTAGCAACTGCAACCGGTG | (Sig Seq- |
| | TACATTCCCAGGTGCAGCTGCGGGAGTCGGGCCCCAGCCTGATGAAGC | VRegion- |
| | CGTCACAGACCCTCTCCCTCACCTGCACGGTCTCTGGATCTTCATTGAA | CH1CH2CH3) |
| | CGACAAGTCTGTAGGCTGGGTCCGCCAGGCTCCAGGGAAGGCGCTGCA | BLV1H12 V in |
| | GTGGCTCGGTAGTGTGGACACTAGTGGAAACACAGACTATAACCCAGG | human IgG |
| | CCTGAAATCCCGGCTCAGCATCACCAAGGACAACTCCAAGAGCCGAAT | |
| | CTCTCTTACAGTGACTGGCATGACAACTGAAGACTCGGCCACATACTA | |
| | CTGTACTTCTGTGCACCAGGAAACAAAAAAATACCAAAGTTGTCCGGA | |
| | GGATTATACTTATAATCCACGTTGCCCTCAGCAGTATGGTTGGAGTGA | |
| | CTGTGATTGTATGGGCGATAGGTTTGGGGGTTACTGTCGACAGGATGG | |
| | TTGTAGTAATTATAGTTATACTTACAATTACGAATGGCACGTCGATGTC | |
| | TGGGGCCAAGGACTCCTGGTCACCGTCTCCTCAGCTAGCACCAAGGGC | |
| | CCATCGGTCTTCCCCCTGGCACCCTCCTCCAAGAGCACCTCTGGGGGC | |
| | ACAGCGGCCCTGGGCTGCCTGGTCAAGGACTACTTCCCCGAACCTGTG | |
| | ACGGTCTCGTGGAACTCAGGCGCCCTGACCAGCGGCGTGCACACCTTC | |
| | CCGGCTGTCCTACAGTCCTCAGGACTCTACTCCCTCAGCAGCGTGGTG | |
| | ACCGTGCCCTCCAGCAGCTTGGGCACCCAGACCTACATCTGCAACGTG | |
| | AATCACAAGCCCAGCAACACCAAGGTGGACAAGAGAGTTGAGCCCAA | |
| | ATCTTGTGACAAAACTCACACATGCCCACCGTGCCCAGCACCTGAACT | |
| | CCTGGGGGGACCGTCAGTCTTCCTCTTCCCCCCAAAACCCAAGGACAC | |
| | CCTCATGATCTCCCGGACCCCTGAGGTCACATGCGTGGTGGTGGACGT | |
| | GAGCCACGAAGACCCTGAGGTCAAGTTCAACTGGTACGTGGACGGCGT | |
| | GGAGGTGCATAATGCCAAGACAAAGCCGCGGGAGGAGCAGTACAACA | |
| | GCACGTACCGTGTGGTCAGCGTCCTCACCGTCCTGCACCAGGACTGGC | |
| | TGAATGGCAAGGAGTACAAGTGCAAGGTCTCCAACAAAGCCCTCCCA | |
| | GCCCCCATCGAGAAAACCATCTCCAAAGCCAAAGGGCAGCCCCGAGA | |
| | ACCACAGGTGTACACCCTGCCCCCATCCCGGGAGGAGATGACCAAGA | |
| | ACCAGGTCAGCCTGACCTGCCTGGTCAAAGGCTTCTATCCCAGCGACA | |
| | TCGCCGTGGAGTGGGAGAGCAATGGGCAGCCGGAGAACAACTACAAG | |
| | ACCACGCCTCCCGTGCTGGACTCCGACGGCTCCTTCTTCCTCTATAGCA | |
| | AGCTCACCGTGGACAAGAGCAGGTGGCAGCAGGGGAACGTCTTCTCAT | |
| | GCTCCGTGATGCATGAGGCTCTGCACAACCACTACACGCAGAAGAGCC | |
| | TCTCCCTGTCCCCGGGTAAATGA | |
| 7 | GAATTCCACCATGGGATGGTCATGTATCATCCTTTTTCTAGTA | B15 variable |
| | GCAACTGCAACCGGAGTACATTCCCAGGTGCAGCTGCGCGAGT | region plus |
| | CGGGCCCCAGCCTGGTGAAGCCGTCACAGACCCTCTCGCTCAC | signal peptide |
| | CTGCACGGCCTCTGGATTCTCATTGAGCGACAAGGCTGTAGGC | |
| | TGGGTCCGCCAGGCTCCAGGGAAGGCGCTGGAGTGGCTCGGTA | |
| | GTATAGACACTGGTGGAAACACAGGCTATAACCCAGGCCTGAA | |
| | ATCCCGGCTCAGCATCACCAAGGACAACTCCAAGAGTCAAGTC | |
| | TCTCTGTCAGTGAGCAGCGTGACAACTGAGGACTCGGCCACAT | |
| | ACTACTGTACTTCTGTGCACCAGGAAACAAAAAAATACCAAAC | |
| | CGGTGGATCAAACTGGGTGAATGTAATAAGTGATTTGAAAAAA | |
| | ATTGAAGATCTTATTCAATCTATGCATATTGATGCTACTTTAT | |
| | ATACGGAAAGTGATGTTCACCCCAGTTGCAAAGTAACAGCAAT | |
| | GAAGTGCTTTCTCTTGGAGTTACAAGTTATTTCACTTGAGTCC | |
| | GGAGATGCAAGTATTCATGATACAGTAGAAAATCTGATCATCC | |
| | TAGCAAACAACAGTTTGTCTTCTAATGGGAATGTAACAGAATC | |
| | TGGATGCAAAGAATGTGAGGAACTGGAGGAAAAAAATATTAAA | |
| | GAATTTTTGCAGAGTTTTGTACATATTGTCCAAATGTTCATCA | |
| | ACACTTCTGGTTCAGGATCCTATACTTACAATTACGAATGGCA | |
| | CGTCGATGTCTGGGGCCAAGGACTCCTGGTCACCGTCTCCTCA | |
| | GCTAGC | |
| 8 | TCA CGA ATT CGC AGG CCG TCC TGA ACC AGC CAA GCA GCG TCT | BLV1H12 Light |
| | CCG GGT CTC TGG GGC AGC GGG TCT CAA TCA CCT GTA GCG GGT | Chain |
| | CTT CCT CCA ATG TCG GCA ACG GCT ACG TGT CTT GGT ATC AGC | |
| | TGA TCC CTG GCA GTG CCC CAC GAA CCC TGA TCT ACG GCG ACA | |
| | CAT CCA GAG CTT CTG GGG TCC CCG ATC GGT TCT CAG GGA GCA | |
| | GAT CCG GAA ACA CAG CTA CTC TGA CCA TCA GCT CCC TGC AGG | |
| | CTG AGG ACG AAG CAG ATT ATT TCT GCG CAT CTG CCG AGG ACT | |
| | CTA GTT CAA ATG CCG TGT TTG GAA GCG GCA CCA CAC TGA CAG | |
| | TCC TGG GGC AGC CCA AGA GTC CCC CTT CAG TGA CTC TGT TCC | |
| | CAC CCT CTA CCG AGG AAC TGA ACG GAA ACA AGG CCA CAC TGG | |
| | TGT GTC TGA TCA GCG ACT TTT ACC CTG GAT CCG TCA CTG TGG | |
| | TCT GGA AGG CAG ATG GCA GCA CAA TTA CTA GGA ACG TGG AAA | |
| | CTA CCC GCG CCT CCA AGC AGT CTA ATA GTA AAT ACG CCG CCA | |
| | GCT CCT ATC TGA GCC TGA CCT CTA GTG ATT GGA AGT CCA AAG | |
| | GGT CAT ATA GCT GCG AAG TGA CCC ATG AAG GCT CAA CCG TGA | |
| | CTA AGA CTG TGA AAC CAT CCG AGT GCT CCT AGG CTA GCT GGC | |
| 9 | TSVHQETKKYQS | BLV1H 12 |
| | | ascending stalk |
| | | region |
| 10 | SYTYNYEWHVDV | BLV1H 12 |
| | | decending stalk |
| | | region |
| 11 | WGQGLLVTVSS | V2 alternative |
| | | sequence |
| 12 | QVQLREWGAGLLKPSETLSLTCAVYGGSFSGYYWSWIRQPPGKGLEWIG | VI Alternative B |
| | EINHSGSTNYNPSLKSRVTISVDTSKNQFSLKLSSVTAADTAVYYC | sequence of |
| | | VH4- |
| | | 34_Q5RQ6E |
| 13 | QVQLREWGAGLLKPSETLSLTCAVYGGSFSDKYWSWIRQPPGKGLEWIG | VI Alternative B |
| | EINHSGSTNYNPSLKSRVTISVDTSKNQFSLKLSSVTAADTAVYYC | sequence of |
| | | VH4-34_CDR1- |
| | | G31DY32KQ5 |
| | | RQ6E |
| 14 | QVQLREWGAGLLKPSETLSLTCAVYGGSFSGYYWSWIRQPPGKGLEWIG | VI Alternative B |
| | SINHSGSTNYNPSLKSRVTISVDTSKNQFSLKLSSVTAADTAVYYC | sequence of |
| | | VH4-34CDR2- |
| | | E50SQ5RQ6E |
| 15 | QVQLREWGAGLLKPSETLSLTCAVYGGSFSDKYWSWIRQPPGKGLEWIG | synthesized: VI |
| | SINHSGSTNYNPSLKSRVTISVDTSKNQFSLKLSSVTAADTAVYYC | Alternative B |
| | | sequence of |
| | | VH4- |
| | | 34CDR1- |
| | | G31DY32KCD |
| | | R2- |
| | | E50SQ5RQ6E |
| 16 | QVQLREWGAGLLKPSETLSLTCTASGFSLSDKAVGWIRQPPGKGLEWIGEI | synthesized: VI |
| | NHSGSTNYNPSLKSRVTISVDTSKNQFSLKLSSVTAADTAVYYC | Alternative B |
| | | sequence of |
| | | VH4-34CDR1- |
| | | Cow_Q5RQ6E |
| 17 | QVQLREWGAGLLKPSETLSLTCAVYGGLGSIDTGGNTGSFSGYYWSWIR | synthesized: VI |
| | QPPGKGLEWYNPSLKSRVTISVDTSKNQFSLKLSSVTAADTAVYYC | Alternative B |
| | | sequence of |
| | | VH4-34CDR2- |
| | | Cow_Q5RQ6E |
| 18 | QVQLREWGAGLLKPSETLSLTCTASGFSLSDKAVGWIRQPPGKGLEWIGSI | synthesized: VI |
| | NHSGSTNYNPSLKSRVTISVDTSKNQFSLKLSSVTAADTAVYYC | Alternative B |
| | | sequence of |
| | | VH4- |
| | | 34CDR1- |
| | | Cow_CDR2- |
| | | E50S_Q5RQ6E |
| 19 | QVQLREWGAGLLKPSETLSLTCTASGFSLSDKAVGWIRQPPGKGLEWLGS | synthesized: VI |
| | IDTGGNTGYNPSLKSRVTISVDTSKNQFSLKLSSVTAADTAVYYC | Alternative N |
| | | sequence of |
| | | VH4- |
| | | 34CDR1- |
| | | Cow_CDR2- |
| | | Cow_Q5RQ6E |
| 20 | WGHGTAVTVSS | V2 alternative |
| | | sequence |
| 21 | WGKGTTVTVSS | V2 alternative |
| | | sequence |
| 22 | WGKGTTVTVSS | V2 alternative |
| | | sequence |
| 23 | WGRGTLVTVSS | V2 alternative |
| | | sequence |
| 24 | WGKGTTVTVSS | V2 alternative |
| | | sequence |
| 25 | SVHQETKKYQSCPDGYRERSDCSNRPACGTSDCCRVSVFGNCLTTLPVSY | Synthesized: |
| | SYTYNYEWHVD | ultralong CDR3 |
| | | sequence |
| | | (BLV1H12) |
| 26 | QVQLRESGPSLVKPSQTLSLTCTASGFSLSDKAVGWVRQAPGKALEWLGS | BLV1H12 |
| | IDTGGNTGYNPGLKSRLSITKDNSKSQVSLSVSSVTTEDSATYYCTSVHQE | Heavy Chain |
| | TKKYQSCPDGYRERSDCSNRPACGTSDCCRVSVFGNCLTTLPVSYSYTYN | |
| | YEWHVDVWGQGLLVTVSSASTTAPKVYPLSSCCGDKSSSTVTLGCLVSS | |
| | YMPEPVTVTWNSGALKSGVHTFPAVLQSSGLYSLSSMVTVPGSTSGQTFT | |
| | CNVAHPASSTKVDKAVEPKSCDGS | |
| 27 | QAVLNQPSSVSGSLGQRVSITCSGSSSNVGNGYVSWYQLIPGSAPRTLIYG | BLV1H12 Light |
| | DTSRASGVPDRFSGSRSGNTATLTISSLQAEDEADYFCASAEDSSSNAVFG | Chain |
| | SGTTLTVLGQPKSPPSVTLFPPSTEELNGNKATLVCLISDFYPGSVTVVWK | |
| | ADGSTITRNVETTRASKQSNSKYAASSYLSLTSSDWKSKGSYSCEVTHEGS | |
| | TVTKTVKPSECS | |
| 28 | QVQLRESGPSLVQPSQTLSLTCTASGFSLSDKAVGWVRQAPGKALEWLGS | BLV5B8 heavy |
| | IDTGGSTGYNPGLKSRLSITKDNSKSQVSLSVSSVTTEDSATYYCTTVHQE | chain |
| | TRKTCSDGYIAVDSCGRGQSDGCVNDCNSCYYGWRNCRRQPAIHSYEFH | |
| | VDAWGRGLLVTVSSASTTAPKVYPLSSCCGDKSSSTVTLGCLVSSYMPEP | |
| | VTVTWNSGALKSGVHTFPAVLQSSGLYSLSSMVTVPGSTS | |
| | GQTFTCNVAHPASSTKVDKAVEPKSCDGS | |
| 29 | QAVLNQPSSVSGSLGQRVSITCSGSSSNVGNGYVSWYQLIPGSAPRTLIYG | BLV5B8 light |
| | DTSRASGVPDRFSGSRSGNTATLTISSLQAEDEADYFCASAEDSSSNAVFG | chain |
| | SGTTLTVLGQPKSPPSVTLFPPSTEELNGNKATLVCLISDFYPGSVTVVWK | |
| | ADGSTITRNVETTRASKQSNSKYAASSYLSLTSSDWKSKGSYSCEVTHEGS | |
| | TVTKTVKPSECS | |
| 30 | TVHQETRKTCSDGYIAVDSCGRGQSDGCVNDCNSCYYGWRNCRRQPAIH | BLV5B8 CDR3 |
| | SYEFHVD | |
| 31 | SVTQRTHVSRSCPDGCSDGDGCVDGCCCSAYRCYTPGVRDLSCTSYSITY | BLV5D3 CDR3 |
| | TYEWNVD | |
| 32 | TVHQKTTRKTCCSDAYRYDSGCGSGCDCCGADCYVFGACTFGLDSSYSY | BLV8C11 CDR3 |
| | IYIYQWYVD | |
| 33 | TVHQIFCPDGYSYGYGCGYGYGCSGYDCYGYGGYGYGGYGGYSSYSYS | BF4E9 CDR3 |
| | YSYEYYGD | |
| 34 | TVHPSPDGYSYGYGCGYGYGCSGYDCYGYGGYGYGGYGGYSSYSYSYS | BF1H1 CDR3 |
| 35 | TVHQIRCPDGYGYGYGCGYGSYGYSGYDCYGYGGYGGYGGYGGYSSYS | F18 CDR3 |
| 36 | TTVHQ | ASCENDING |
| | | STALK |
| | | STRAND |
| 37 | TSVHQ | ASCENDING |
| | | STALK |
| | | STRAND |
| 38 | SSVTQ | ASCENDING |
| | | STALK |
| | | STRAND |
| 39 | STVHQ | ASCENDING |
| | | STALK |
| | | STRAND |
| 40 | ATVRQ | ASCENDING |
| | | STALK |
| | | STRAND |
| 41 | TTVYQ | ASCENDING |
| | | STALK |
| | | STRAND |
| 42 | SPVHQ | ASCENDING |
| | | STALK |
| | | STRAND |
| 43 | ATVYQ | ASCENDING |
| | | STALK |
| | | STRAND |
| 44 | TAVYQ | ASCENDING |
| | | STALK |
| | | STRAND |
| 45 | TNVHQ | ASCENDING |
| | | STALK |
| | | STRAND |
| 46 | ATVHQ | ASCENDING |
| | | STALK |
| | | STRAND |
| 47 | STVYQ | ASCENDING |
| | | STALK |
| | | STRAND |
| 48 | TIVHQ | ASCENDING |
| | | STALK |
| | | STRAND |
| 49 | AIVYQ | ASCENDING |
| | | STALK |
| | | STRAND |
| 50 | TTVFQ | ASCENDING |
| | | STALK |
| | | STRAND |
| 51 | AAVFQ | ASCENDING |
| | | STALK |
| | | STRAND |
| 52 | GTVHQ | ASCENDING |
| | | STALK |
| | | STRAND |
| 53 | ASVHQ | ASCENDING |
| | | STALK |
| | | STRAND |
| 54 | TAVFQ | ASCENDING |
| | | STALK |
| | | STRAND |
| 55 | ATVFQ | ASCENDING |
| | | STALK |
| | | STRAND |
| 56 | AAAHQ | ASCENDING |
| | | STALK |
| | | STRAND |
| 57 | VWYQ | ASCENDING |
| | | STALK |
| | | STRAND |
| 58 | GTVFQ | ASCENDING |
| | | STALK |
| | | STRAND |
| 59 | TAVHQ | ASCENDING |
| | | STALK |
| | | STRAND |
| 60 | ITVHQ | ASCENDING |
| | | STALK |
| | | STRAND |
| 61 | ITAHQ | ASCENDING |
| | | STALK |
| | | STRAND |
| 62 | VTVHQ | ASCENDING |
| | | STALK |
| | | STRAND |
| 63 | AAV HQ | ASCENDING |
| | | STALK |
| | | STRAND |
| 64 | GTVYQ | ASCENDING |
| | | STALK |
| | | STRAND |
| 65 | TTVLQ | ASCENDING |
| | | STALK |
| | | STRAND |
| 66 | TTTHQ | ASCENDING |
| | | STALK |
| | | STRAND |
| 67 | TTDYQ | ASCENDING |
| | | STALK |
| | | STRAND |
| 68 | QLQLQESGPGLVKPSETLSLTCTVSGGSISSSSYYWGWIRQPPGKGLEWIG | Human heavy |
| | SIYYSGSTYYNPSLKSRVTISVDTSKNQFSLKLSSVTAADTAVYYCAR | chain variable |
| | | region sequence |
| | | VH4-39 |
| 69 | QVQLQESGPGLVKPSETLSLTCTVSGGSISSYYWSWIRQPPGKGLEWIGYI | Human heavy |
| | YYSGSTNYNPSLKSRVTISVDTSKNQFSLKLSSVTAADTAVYYCA | chain variable |
| | | region sequence |
| | | 4-59*03 |
| 70 | QVQLQQWGAGLLKPSETLSLTCAVYGGSFSGYYWSWIRQPPGKGLEWIG | Human heavy |
| | EINHSGSTNYNPSLKSRVTISVDTSKNQFSLKLSSVTAADTAVYYCAR | chain variable |
| | | region sequence |
| | | 4-34*02 |
| 71 | QVQLQESGPGLVKPSQTLSLTCAVYGGSFSGYYWSWIRQPPGKGLEWIGE | Human heavy |
| | INHSGSTNYNPSLKSRVTISVDTSKNQFSLKLSSVTAADTAVYYCAR | chain variable |
| | | region sequence |
| | | 4-34*09 |
| 72 | TSVHQETKKYQ | ASCENDING |
| | | STALK |
| | | STRAND |
| 73 | VHQETKKYQ | ASCENDING |
| | | STALK |
| | | STRAND |
| 74 | IHSYEF | ASCENDING |
| | | STALK |
| | | STRAND |
| 75 | SYEF | ASCENDING |
| | | STALK |
| | | STRAND |
| 76 | YTYNYE | DESCENDING |
| | | STALK |
| | | STRAND |
| 77 | YTYNYEW | DESCENDING |
| | | STALK |
| | | STRAND |
| 78 | SYTYNYEW | DESCENDING |
| | | STALK |
| | | STRAND |
| 79 | TYNYEW | DESCENDING |
| | | STALK |
| | | STRAND |
| 80 | SYTY | DESCENDING |
| | | STALK |
| | | STRAND |
| 81 | GSKHRLRDYFLYNE | ASCENDING |
| | | STALK |
| | | STRAND |
| 82 | GSKHRLRDYFLYN | ASCENDING |
| | | STALK |
| | | STRAND |
| 83 | GSKHRLRDYFLY | ASCENDING |
| | | STALK |
| | | STRAND |
| 84 | GSKHRLRDYFL | ASCENDING |
| | | STALK |
| | | STRAND |
| 85 | GSKHRLRDYF | ASCENDING |
| | | STALK |
| | | STRAND |
| 86 | GSKHRLRDY | ASCENDING |
| | | STALK |
| | | STRAND |
| 87 | GSKHRIRD | ASCENDING |
| | | STALK |
| | | STRAND |
| 88 | EAGGPDYRNGYNY | ASCENDING |
| | | STALK |
| | | STRAND |
| 89 | EAGGPDYRNGYN | ASCENDING |
| | | STALK |
| | | STRAND |
| 90 | EAGGPDYRNGY | ASCENDING |
| | | STALK |
| | | STRAND |
| 91 | EAGGPDYRNG | ASCENDING |
| | | STALK |
| | | STRAND |
| 92 | EAGGPDYRN | ASCENDING |
| | | STALK |
| | | STRAND |
| 93 | EAGGPDYR | ASCENDING |
| | | STALK |
| | | STRAND |
| 94 | EAGGPDY | ASCENDING |
| | | STALK |
| | | STRAND |
| 95 | EAGGPD | ASCENDING |
| | | STALK |
| | | STRAND |
| 96 | EAGGPI WHDDVKY | ASCENDING |
| | | STALK |
| | | STRAND |
| 97 | EAGGPIWHDDVK | ASCENDING |
| | | STALK |
| | | STRAND |
| 98 | EAGGPIWHDDV | ASCENDING |
| | | STALK |
| | | STRAND |
| 99 | EAGGPIWHDD | ASCENDING |
| | | STALK |
| | | STRAND |
| 100 | EAGGPIWHD | ASCENDING |
| | | STALK |
| | | STRAND |
| 101 | EAGGPIWH | ASCENDING |
| | | STALK |
| | | STRAND |
| 102 | EAGGPIW | ASCENDING |
| | | STALK |
| | | STRAND |
| 103 | EAGGPI | ASCENDING |
| | | STALK |
| | | STRAND |
| 104 | GTDYTIDDQGI | ASCENDING |
| | | STALK |
| | | STRAND |
| 105 | GTDYTIDDQG | ASCENDING |
| | | STALK |
| | | STRAND |
| 106 | GTDYTIDDQ | ASCENDING |
| | | STALK |
| | | STRAND |
| 107 | GTDYTIDD | ASCENDING |
| | | STALK |
| | | STRAND |
| 108 | GTDYTID | ASCENDING |
| | | STALK |
| | | STRAND |
| 109 | GTDYTI | ASCENDING |
| | | STALK |
| | | STRAND |
| 110 | DKGDSDYDYNL | ASCENDING |
| | | STALK |
| | | STRAND |
| ill | DKGDSDYDYN | ASCENDING |
| | | STALK |
| | | STRAND |
| 112 | DKGDSDYDY | ASCENDING |
| | | STALK |
| | | STRAND |
| 113 | DKGDSDYD | ASCENDING |
| | | STALK |
| | | STRAND |
| 114 | DKGDSDY | ASCENDING |
| | | STALK |
| | | STRAND |
| 115 | DKGDSD | ASCENDING |
| | | STALK |
| | | STRAND |
| 116 | YGPNYEEWGDYLATLDV | ASCENDING |
| | | STALK |
| | | STRAND |
| 117 | GPNYEEWGDYLATLDV | ASCENDING |
| | | STALK |
| | | STRAND |
| 118 | PNYEEWGDYLATLDV | ASCENDING |
| | | STALK |
| | | STRAND |
| 119 | NYEEWGDYLATLDV | ASCENDING |
| | | STALK |
| | | STRAND |
| 120 | YEEWGDYLATLDV | ASCENDING |
| | | STALK |
| | | STRAND |
| 121 | EEWGDYLATLDV | ASCENDING |
| | | STALK |
| | | STRAND |
| 122 | YDFYDGYYNYHYMDV | DESCENDING |
| | | STALK |
| | | STRAND |
| 123 | DFYDGYYNYHYMDV | DESCENDING |
| | | STALK |
| | | STRAND |
| 124 | FYDGYYNYHYMDV | DESCENDING |
| | | STALK |
| | | STRAND |
| 125 | YDGYYNYHYMDV | DESCENDING |
| | | STALK |
| | | STRAND |
| 126 | DGYYNYHYMDV | DESCENDING |
| | | STALK |
| | | STRAND |
| 127 | GYYNYHYMDV | DESCENDING |
| | | STALK |
| | | STRAND |
| 128 | YYNYHYMDV | DESCENDING |
| | | STALK |
| | | STRAND |
| 129 | YDFNDGYYNYHYMDV | DESCENDING |
| | | STALK |
| | | STRAND |
| 130 | DFYDGYYNYHYMDV | DESCENDING |
| | | STALK |
| | | STRAND |
| 131 | FYDGYYNYHYMDV | DESCENDING |
| | | STALK |
| | | STRAND |
| 132 | YDGYYNYHYMDV | DESCENDING |
| | | STALK |
| | | STRAND |
| 133 | DGYYNYHYMDV | DESCENDING |
| | | STALK |
| | | STRAND |
| 134 | GYYNYHYMDV | DESCENDING |
| | | STALK |
| | | STRAND |
| 135 | QGIRYQGSGTFWYFDV | DESCENDING |
| | | STALK |
| | | STRAND |
| 136 | GIRYQGSGTFWYFDV | DESCENDING |
| | | STALK |
| | | STRAND |
| 137 | IRYQGSGTFWYFDV | DESCENDING |
| | | STALK |
| | | STRAND |
| 138 | RYQGSGTFWYFDV | DESCENDING |
| | | STALK |
| | | STRAND |
| 139 | YQGSGTFWYFDV | DESCENDING |
| | | STALK |
| | | STRAND |
| 140 | QGSGTFWYFDV | DESCENDING |
| | | STALK |
| | | STRAND |
| 141 | GSGTFWYFDV | DESCENDING |
| | | STALK |
| | | STRAND |
| 142 | SGTFWYFDV | DESCENDING |
| | | STALK |
| | | STRAND |
| 143 | GTFWYFDV | DESCENDING |
| | | STALK |
| | | STRAND |
| 144 | YNLGYSYFYYMDG | DESCENDING |
| | | STALK |
| | | STRAND |
| 145 | NLGYSYFYYMDG | DESCENDING |
| | | STALK |
| | | STRAND |
| 146 | LGYSY FYYMDG | DESCENDING |
| | | STALK |
| | | STRAND |
| 147 | GYSYFYYMDG | DESCENDING |
| | | STALK |
| | | STRAND |
| 148 | YSYFYYMDG | DESCENDING |
| | | STALK |
| | | STRAND |
| 149 | SYFYYMDG | DESCENDING |
| | | STALK |
| | | STRAND |
| 150 | GS | Linker |
| 151 | GGS | Linker |
| 152 | GGSGGS | Linker |
| 153 | GGSGGSGGS | Linker |
| 154 | GGGGS | Linker |
| 155 | tcacgaattc gcaggccgtc ctgaaccagc caagcagcgt ctccgggtct ctggggcagc | human light |
| | gggtctcaat cacctgtagc gggtcttcct ccaatgtcgg caacggctac gtgtcttggt | chain lambda |
| | atcagctgat ccctggcagt gccccacgaa ccctgatcta cggcgacaca tccagagctt | region |
| | ctggggtccc cgatcggttc tcagggagca gatccggaaa cacagctact ctgaccatca | |
| | gctccctgca ggctgaggac gaagcagatt atttctgcgc atctgccgag gactctagtt | |
| | caaatgccgt gtttggaagc ggcaccacac tgacagtcct aggtcagccc aaggctgccc | |
| | cctcggtcac tctgttcccg ccctcctctg aggagcttca agccaacaag gccacactgg | |
| | tgtgtctcat aagtgacttc tacccgggag ccgtgacagt ggcctggaag gcagatagca | |
| | gccccgtcaa ggcgggagtg gagaccacca caccctccaa acaaagcaac aacaagtacg | |
| | cggccagcag ctatctgagc ctgacgcctg agcagtggaa gtcccacaga agctacagct | |
| | gccaggtcac gcatgaaggg agcaccgtgg agaagacagt ggcccctaca gaatgttcat | |
| | aa | |
| 156 | QSVLTQPPSVSAAPGQKVTISCSGSSSNIGNNYVSWYQQLPGTAPKLLIYD | human VL1-51 |
| | NNKRPSGIPDRFSGSKSGTSATLGITGLQTGDEADYYCASAEDSSSNAVFG | |
| | SGTTLTVLGQPKAAPSVTLFPPSSEELQANKATLVCLISDFYPGAVTVAWK | |
| | ADSSPVKAGVETTTPSKQSNNKYAASSYLSLTPEQWKSHRSYSCQVTHEG | |
| | STVEKTVAPTECS | |
| 157 | TGTACTTCTGTGCACCAGGAAACAAAAAAATACCAAACC | BLVIH12 |
| | | ascending stalk |
| | | region |
| 158 | TSVHQETKKYQT | BLVIH12 |
| | | ascending stalk |
| | | region |
| 159 | CTSVHQETKKYQT | BLVIH12 |
| | | ascending stalk |
| | | region |
| 160 | TCCTATACTTACAATTACGAATGGCACGTCGATGTCTGG | Descending stalk |
| | | region |
| 161 | SYTYNYEWHVDVW | Descending stalk |
| | | region |
| 162 | TGTCCGGAGGATTATACTTATAATCCACGTTGCCCTCAGCAGTATGGTT | BLVIH12 knob |
| | GGAGTGACTGTGATTGTATGGGCGATAGGTTTGGGGGTTACTGTCGAC | sequence |
| | AGGATGGTTGTAGTAATTAT | |
| 163 | ggtggatca | Coding |
| | | sequencing for |
| | | N-terminal GGS |
| | | linker |
| 164 | ggttcagga | Coding sequence |
| | | for C-terminal |
| | | GSG linker |
| 165 | APTSSSTKKTQLQLEHLLLDLQMILNGINNYKNPKLTRMLTFKFYMPKKA | IL2 |
| | TELKHLQCLEEELKPLEEVLNLAQSKNFHLRPRDLISNINVIVLELKGSETT | |
| | FMCEYADETATIVEFLNRWITFCQSIISTLT | |
| 166 | GCACCTACTTCAAGTTCTACAAAGAAAACACAGCTACAACTGGAGCAT | IL2 coding |
| | TTACTGCTGGATTTACAGATGATTTTGAATGGAATTAATAATTACAAG | sequence |
| | AATCCCAAACTCACCAGGATGCTCACATTTAAGTTTTACATGCCCAAG | |
| | AAGGCCACAGAACTGAAACATCTTCAGTGTCTAGAAGAAGAACTCAA | |
| | ACCTCTGGAGGAAGTGCTAAATTTAGCTCAAAGCAAAAACTTTCACTT | |
| | AAGACCCAGGGACTTAATCAGCAATATCAACGTAATAGTTCTGGAACT | |
| | AAAGGGATCTGAAACAACATTCATGTGTGAATATGCTGATGAGACAGC | |
| | AACCATTGTAGAATTTCTGAACAGATGGATTACCTTTTGTCAAAGCATC | |
| | ATCTCAACACTGACT | |
| 167 | CAGGTGCAGCTGCGGGAGTCGGGCCCCAGCCTGATGAAGCCGTCACA | Chimeric |
| | GACCCTCTCCCTCACCTGCACGGTCTCTGGATCTTCATTGAACGACAAG | ultralong bovine |
| | TCTGTAGGCTGGGTCCGCCAGGCTCCAGGGAAGGCGCTGCAGTGGCTC | heavy chain |
| | GGTAGTGTGGACACTAGTGGAAACACAGACTATAACCCAGGCCTGAA | sequence |
| | ATCCCGGCTCAGCATCACCAAGGACAACTCCAAGAGCCGAATCTCTCT | |
| | TACAGTGACTGGCATGACAACTGAAGACTCGGCCACATACTACTGTAC | |
| | TTCTGTGCACCAGGAAACAAAAAAATACCAAAGTTGTCCGGAGGATTA | |
| | TACTTATAATCCACGTTGCCCTCAGCAGTATGGTTGGAGTGACTGTGAT | |
| | TGTATGGGCGATAGGTTTGGGGGTTACTGTCGACAGGATGGTTGTAGT | |
| | AATTATAGTTATACTTACAATTACGAATGGCACGTCGATGTCTGGGGC | |
| | CAAGGACTCCTGGTCACCGTCTCCTCAGCTAGC | |
| 168 | CAGGCCGTCCTGAACCAGCCAAGCAGCGTCTCCGGGTCTCTGGGGCAG | BLVIH12 Light |
| | CGGGTCTCAATCACCTGTAGCGGGTCTTCCTCCAATGTCGGCAACGGC | Chain- |
| | TACGTGTCTTGGTATCAGCTGATCCCTGGCAGTGCCCCACGAACCCTG | |
| | ATCTACGGCGACACATCCAGAGCTTCTGGGGTCCCCGATCGGTTCTCA | |
| | GGGAGCAGATCCGGAAACACAGCTACTCTGACCATCAGCTCCCTGCAG | |
| | GCTGAGGACGAAGCAGATTATTTCTGCGCATCTGCCGAGGACTCTAGT | |
| | TCAAATGCCGTGTTTGGAAGCGGCACCACACTGACAGTCCTAGGTCAG | |
| | CCCAAGGCTGCCCCCTCGGTCACTCTGTTCCCGCCCTCCTCTGAGGAGC | |
| | TTCAAGCCAACAAGGCCACACTGGTGTGTCTCATAAGTGACTTCTACC | |
| | CGGGAGCCGTGACAGTGGCCTGGAAGGCAGATAGCAGCCCCGTCAAG | |
| | GCGGGAGTGGAGACCACCACACCCTCCAAACAAAGCAACAACAAGTA | |
| | CGCGGCCAGCAGCTATCTGAGCCTGACGCCTGAGCAGTGGAAGTCCCA | |
| | CAGAAGCTACAGCTGCCAGGTCACGCATGAAGGGAGCACCGTGGAGA | |
| | AGACAGTGGCCCCTACAGAATGTTCATAA | |
| 169 | cagctgcagc tgcaggagtc gggcccagga ctggtgaagc | Human heavy |
| | cttcggagac cctgtccctc acctgcactg tctctggtgg | chain variable |
| | ctccatcagc agtagtagtt actactgggg ctggatccgc | region sequence |
| | cagcccccag ggaaggggct ggagtggatt gggagtatct | 4-39 |
| | attatagtgg gagcacctac tacaacccgt ccctcaagag | |
| | tcgagtcacc atatccgtag acacgtccaa gaaccagttc | |
| | tccctgaagc tgagctctgt gaccgccgca gacacggctg | |
| | tgtattactg tgcgagacac acagtgaggg g | |
| 170 | caggtgcagc tgcaggagtc gggcccagga ctggtgaagc | Human heavy |
| | cttcggagac cctgtccctc acctgcactg tctctggtgg | chain variable |
| | ctccatcagt agttactact ggagctggat ccggcagccc | region sequence |
| | ccagggaagg gactggagtg gattgggtat atctattaca | 4-59*03 |
| | gtgggagcac caactacaac ccctccctca agagtcgagt | |
| | caccatatca gtagacacgt ccaagaacca attctccctg | |
| | aagctgagct ctgtgaccgc tgcggacacg gccgtgtatt | |
| | actgtgcg | |
| 171 | caggtgcagc tgcaggagtc gggcccagga ctggtgaagc cttcacagac cctgtccctc | Human heavy |
| | acctgcgctg tctatggtgg gtccttcagt ggttactact ggagctggat ccgccagccc | chain variable |
| | ccagggaagg gactggagtg gattggggaa atcaatcata gtggaagcac caactacaac | region sequence |
| | ccgtccctca agagtcgagt taccatatca gtagacacgt ctaagaacca gttctccctg | 4-34*09 |
| | aagctgagct ctgtgactgc cgcggacacg gccgtgtatt actgtgcgag a | |
| 172 | caggtgcagc tacaacagtg gggcgcagga ctgttgaagc cttcggagac cctgtccctc | Human heavy |
| | acctgcgctg tctatggtgg gtccttcagt ggttactact ggagctggat ccgccagccc | chain variable |
| | ccagggaagg ggctggagtg gattggggaa atcaatcata gtggaagcac caactacaac | region sequence |
| | ccgtccctca agagtcgagt caccatatca gtagacacgt ccaagaacca gttctccctg | 4-34*02 |
| | aagctgagct ctgtgaccgc cgcggacacg gctgtgtatt actgtgcgag | |
| 173 | QSVLTQPPSASGTPGQRVTISCSGSSSNIGSNYVYWYQQLPGTAPKLLIYR | Human germline |
| | NNQRPSGVPDRFSGSKSGTSASLAISGLRSEDEADYYCAAWDDSLSG | light chain |
| | | variable region |
| | | sequence VL1- |
| | | 47 |
| 174 | QSVLTQPPSVSGAPGQRVTISCTGSSSNIGAGYDVHWYQQLPGTAPKLLIY | Human germline |
| | GNSNRPSGVPDRFSGSKSGTSASLAITGLQAEDEADYYCQSYDSSLSG | light chain |
| | | variable region |
| | | sequence VL1- |
| | | 40*1 |
| 175 | QSVLTQPPSVSAAPGQKVTISCSGSSSNIGNNYVSWYQQLPGTAPKLLIYD | Human germline |
| | NNKRPSGIPDRFSGSKSGTSATLGITGLQTGDEADYYCGTWDSSLSA | light chain |
| | | variable region |
| | | sequence VL1- |
| | | 51*01 |
| 176 | QSALTQPPSVSGSPGQSVTISCTGTSSDVGSYNRVSWYQQPPGTAPKLMIY | Human germline |
| | EVSNRPSGVPDRFSGSKSGNTASLTISGLQAEDEADYYCSSYTSSSTF | light chain |
| | | variable region |
| | | sequence VL2- |
| | | 18*02 |
| 177 | cagtctgtgc tgactcagcc accctcagcg tctgggaccc ccgggcagag ggtcaccatc | Human germline |
| | tcttgttctg gaagcagctc caacatcgga agtaattatg tatactggta ccagcagctc | light chain |
| | ccaggaacgg cccccaaact cctcatctat aggaataatc agcggccctc aggggtccct | variable region |
| | gaccgattct ctggctccaa gtctggcacc tcagcctccc tggccatcag tgggctccgg | sequence VL1- |
| | tccgaggatg aggctgatta ttactgtgca gcatgggatg acagcctgag tggtcc | 47 |
| 178 | cagtctgtgc tgacgcagcc gccctcagtg tctggggccc cagggcagag ggtcaccatc | Human germline |
| | tcctgcactg ggagcagctc caacatcggg gcaggttatg atgtacactg gtaccagcag | light chain |
| | cttccaggaa cagcccccaa actcctcatc tatggtaaca gcaatcggcc ctcaggggtc | variable region |
| | cctgaccgat tctctggctc caagtctggc acctcagcct ccctggccat cactgggctc | sequence VL1- |
| | caggctgagg atgaggctga ttattactgc cagtcctatg acagcagcct gagtggttc | 40*1 |
| 179 | cagtctgtgt tgacgcagcc gccctcagtg tctgcggccc caggacagaa ggtcaccatc | Human germline |
| | tcctgctctg gaagcagctc caacattggg aataattatg tatcctggta ccagcagctc | light chain |
| | ccaggaacag cccccaaact cctcatttat gacaataata agcgaccctc agggattcct | variable region |
| | gaccgattct ctggctccaa gtctggcacg tcagccaccc tgggcatcac cggactccag | sequence VL1- |
| | actggggacg aggccgatta ttactgcgga acatgggata gcagcctgag tgctgg | 51*01 |
| 180 | cagtctgccc tgactcagcc tccctccgtg tccgggtctc ctggacagtc agtcaccatc | Human germline |
| | tcctgcactg gaaccagcag tgacgttggt agttataacc gtgtctcctg gtaccagcag | light chain |
| | cccccaggca cagcccccaa actcatgatt tatgaggtca gtaatcggcc ctcaggggtc | variable region |
| | cctgatcgct tctctgggtc caagtctggc aacacggcct ccctgaccat ctctgggctc | sequence VL2- |
| | caggctgagg acgaggctga ttattactgc agctcatata caagcagcag cactttc | 18*02 |

What is claimed:

1. A chimeric cytokine modified antibody or antigen binding fragment, comprising a modified ultralong CDR3 comprising an interleukin-15 (IL-15) cytokine sequence or a biologically active portion thereof that replaces at least a portion of an ultralong CDR3 region of a heavy chain of a bovine antibody or antigen-binding fragment or a humanized sequence thereof.

2. The chimeric cytokine modified antibody or antigen binding fragment of claim 1, wherein the IL-15 cytokine sequence is human IL-15.

3. The chimeric cytokine modified antibody or antigen binding fragment of claim 1 or claim 2, wherein the IL-15 cytokine sequence comprises a sequence of amino acids that exhibits at least at or about 85%, at least at or about 90%, at least at or about 92%, at least at or about 95%, at least at or about 96%, at least at or about 97%, at least at or about 98%, or at least at or about 99% sequence identity to SEQ ID NO:1.

4. The chimeric cytokine modified antibody or antigen binding fragment of any of claims 1-3 wherein the IL-15 cytokine sequence comprises the sequence of amino acids set forth in SEQ ID NO:1.

5. A chimeric cytokine modified antibody or antigen binding fragment, comprising a modified ultralong CDR3 comprising an interleukin-2 (IL-2) cytokine sequence or a biologically active portion thereof that replaces at least a portion of an ultralong CDR3 region of a heavy chain of a bovine antibody or antigen-binding fragment or a humanized sequence thereof.

6. The chimeric cytokine modified antibody or antigen binding fragment of claim 5, wherein the IL-2 cytokine sequence is human IL-2.

7. The chimeric cytokine modified antibody or antigen binding fragment of claim 5 or claim 6, wherein the IL-2 cytokine sequence comprises a sequence of amino acids that exhibits at least at or about 85%, at least at or about 90%, at least at or about 92%, at least at or about 95%, at least at or about 96%, at least at or about 97%, at least at or about 98%, or at least at or about 99% sequence identity to SEQ ID NO:165.

8. The chimeric cytokine modified antibody or antigen binding fragment of any of claims 5-7 wherein the IL-2 cytokine sequence comprises the sequence of amino acids set forth in SEQ ID NO:165.

9. The chimeric cytokine modified antibody or antigen binding fragment of any of claims 1-8, wherein the cytokine sequence replaces at least a portion of an ultralong CDR3 region of a heavy chain of a bovine antibody or antigen-binding fragment.

10. The chimeric cytokine modified antibody or antigen binding fragment of claim 9, wherein the bovine antibody or antigen-binding fragment is the bovine antibody BLV1H12 or an antigen-binding fragment thereof.

11. The chimeric cytokine modified antibody or antigen binding fragment of claim 9 or claim 10, wherein the bovine antibody or antigen-binding fragment comprises a variable heavy chain amino acid sequence encoded by the sequence set forth in SEQ ID NO:5 and a variable light chain amino acid sequence encoded by the sequence set forth in SEQ ID NO: 8.

12. The chimeric cytokine modified antibody or antigen binding fragment of claim 9 or claim 10, wherein the bovine antibody or antigen-binding fragment comprises a variable heavy chain amino acid sequence encoded by the sequence set forth in SEQ ID NO: 167 and a variable light chain amino acid sequence encoded by the sequence set forth in SEQ ID NO:168.

13. The chimeric cytokine modified antibody or antigen binding fragment of claim 9 or claim 10, wherein the bovine antibody or antigen-binding fragment comprises a variable heavy chain set forth in SEQ ID NO: 26 and a variable light chain set forth in SEQ ID NO: 27.

14. The chimeric cytokine modified antibody or antigen binding fragment of any of claims 1-8, wherein the cytokine sequence replaces at least a portion of an ultralong CDR3 region of a heavy chain of a humanized bovine antibody or antigen-binding fragment thereof.

15. The chimeric cytokine modified antibody or antigen binding fragment of claim 14, wherein the humanized bovine antibody or antigen-binding fragment thereof comprises a heavy chain or portion thereof that is a human heavy chain germline sequence or is derived from a human heavy chain germline sequence and a light chain or a portion thereof that is a human light chain germline sequence or is derived from a human light chain germline sequence.

16. The chimeric cytokine modified antibody or antigen binding fragment of claim 15, wherein the human heavy chain germline sequence is a VH4-39, VH4-59*03, VH4-34*02 or VH4-34*09 germline sequence or is a sequence set forth in any one of SEQ ID NOS: 68-71.

17. The chimeric cytokine modified antibody or antigen binding fragment of claim 15 or claim 16, wherein the human light chain germline sequence is a VL1-51 germline sequence or is a sequence based on the VL1-51 germline sequence comprising one or more mutations, optionally wherein the VL1-51 germline sequence is set forth in SEQ ID NO:156.

18. The chimeric cytokine modified antibody or antigen binding fragment of claim 17, wherein the one or more mutations are selected from among:
one or more of amino acid replacements S2A, T5N, P8S, A12G, A13S, and P14L based on Kabat numbering;
amino acid replacements S2A, T5N, P8S, A12G, A13S, and P14L based on Kabat numbering;
mutations in CDR1 comprising amino acid replacements 129V and N32G;
mutations in CDR2 comprising a substitution of DNN to GDT;
mutations in CDR2 comprising a substitution DNNKRP to GDTSRA;
or a combination of any of the forgoing.

19. The chimeric cytokine modified antibody or antigen binding fragment of any of claims 1-18, wherein the antibody is an antigen-binding fragment comprising a variable heavy chain and a variable light chain.

20. The chimeric cytokine modified antibody or antigen binding fragment of any of claims 1-19, wherein the antibody comprises a variable heavy chain joined to a heavy chain constant domain (CH1-CH2-CH3) and a variable light chain joined to a light chain constant domain (CL1).

21. The chimeric cytokine modified antibody or antigen binding fragment of claim 20, wherein the heavy chain constant domain is from a human IgG1.

22. The chimeric cytokine modified antibody or antigen binding fragment of claim 20 or claim 21, wherein the light chain constant domain is a lambda light chain region.

23. The chimeric cytokine modified antibody or antigen binding fragment of any of claims 1-22, wherein the at least a portion of an ultralong CDR3 region comprises the knob region and the cytokine sequence is present between the ascending stalk domain and the descending stalk domain of the modified ultralong CDR3.

24. The chimeric cytokine modified antibody or antigen binding fragment of claim 23, wherein the cytokine sequence is linked to the ascending stalk domain and/or the descending stalk domain via a flexible linker, optionally a GGS or GSG linker.

25. The chimeric cytokine modified antibody or antigen binding fragment of claim 23 or claim 24, wherein the ascending stalk domain comprises the sequence set forth in SEQ ID NO: 158 or SEQ ID NO:159.

26. The chimeric cytokine modified antibody or antigen binding fragment of any of claims 23-25, wherein the descending stalk domain comprises the sequence set forth in SEQ ID NO:161.

27. The chimeric cytokine modified antibody or antigen binding fragment of any of claims 1-4 and 9-26, wherein the antibody or antigen binding fragment comprises a variable heavy chain sequence encoded by the sequence of nucleotides set forth in SEQ ID NO:7 or a sequence of nucleotides that exhibits at least at or about 85%, at least at or about 90%, at least at or about 92%, at least at or about 95%, at least at or about 96%, at least at or about 97%, at least at or about 98%, at least at or about 99% sequence identity to the nucleotide sequence set forth in SEQ ID NO:7, in which is contained a modified ultralong CDR3 containing an IL-15 sequence.

28. The chimeric cytokine modified antibody or antigen binding fragment of any of claims 1-4 and 9-27, wherein the antibody or antigen binding fragment is complexed with an extracellular domain of the IL15Rα comprising the IL15Rα sushi domain.

29. The chimeric cytokine modified antibody or antigen binding fragment of claim 28, wherein the extracellular domain of the IL15Rα comprising the IL15Rα sushi domain is non-covalently associated with the IL-15 sequence.

30. The chimeric cytokine modified antibody or antigen binding fragment of claim 28, wherein the extracellular domain of the IL15Rα comprising the IL15Rα sushi domain is linked to the variable light chain.

31. The chimeric cytokine modified antibody or antigen binding fragment of claim 30 that is linked via a peptide linker.

32. The chimeric cytokine modified antibody of claim 31, wherein the peptide linker is a glycine linker or a glycine-serine linker, optionally wherein the linker is GS.

33. The chimeric cytokine modified antibody of any of claims 28-32, wherein the extracellular domain of the IL15Rα comprising the IL15Rα sushi domain comprises the sequence set forth in SEQ ID NO:2.

34. The chimeric cytokine modified antibody or antigen binding fragment of any of claims 30-33, wherein the variable light chain comprises the sequence of amino acids encoded by SEQ ID NO:3.

35. A polynucleotide(s) encoding a chimeric cytokine modified antibody or antigen binding fragment of any of claims 1-34.

36. A polynucleotide encoding a heavy chain or a variable region thereof of a chimeric cytokine modified antibody or antigen binding fragment of any of claims 1-34.

37. A polynucleotide encoding a light chain or a variable region thereof of a chimeric cytokine modified antibody or antigen binding fragment of any of claims 1-34.

38. An expression vector comprising the polynucleotide of any of claims 35-37.

39. A host cell comprising the polynucleotide of any of claims 35-37 or the expression vector of claim 37.

40. The host cell of claim 39, further comprising a polynucleotide or vector expressing an extracellular domain of the IL15Rα comprising the IL15Rα sushi domain.

41. The host cell of claim 40, wherein the extracellular domain of the IL15Rα comprising the IL15Rα sushi domain comprises the sequence set forth in SEQ ID NO:2.

42. A method of producing a chimeric cytokine modified antibody or antigen binding fragment comprising culturing the host cell of any of claims 39-41 under conditions for expression of the antibody or antigen binding fragment by the cell, optionally further comprising recovering of purifying the antibody or antigen binding fragment.

43. A chimeric cytokine modified antibody or antigen binding fragment produced by the method of claim 42.

44. A pharmaceutical composition comprising the chimeric cytokine modified antibody or antigen binding fragment of any of claims 1-34 or 43.

45. A method of treating a cancer in a subject, comprising administering a therapeutically effective amount of a chimeric cytokine modified antibody or antigen binding fragment of any of claims 1-34 or 43.

46. A method of treating a cancer in a subject, comprising administering a therapeutically effective amount of a pharmaceutical composition of claim 44.
